(19) United States
(12) Patent Application Publication
(10) Pub. No.: US 2024/0344132 A1
(43) Pub. Date: Oct. 17, 2024

(54) Method of Treating Liver Cancer, Predicting Response to Treatment, and Predicting Adverse Effects During the Treatment Thereof

(71) Applicant: Singapore Health Services Pte Ltd, Singapore (SG)

(72) Inventors: Suk Peng, Valerie Chew, Singapore (SG); Wai-Meng, David Tai, Singapore (SG); Salvatore Albani, Singapore (SG); Wen Jin, Samuel Chuah, Singapore (SG)

(21) Appl. No.: 18/134,125

(22) Filed: Apr. 13, 2023

(51) Int. Cl. C12Q 1/6886; C12Q 1/6869; G01N 33/569; G01N 33/574

(52) U.S. Cl. CPC C12Q 1/6886; C12Q 1/6869; G01N 33/56972; G01N 33/57484; C12Q 2600/158

(57) ABSTRACT

This technology relates to a method of treating a liver cancer, and a method of predicting a response of a subject suffering from liver cancer to a treatment with an immune checkpoint inhibitor. This technology further relates to a method of predicting a treatment-induced immune-related adverse events (irAEs) of a subject suffering from liver cancer to a treatment with an immune checkpoint inhibitor.

### FIELD OF THE INVENTION

The present invention relates generally to the field of cell biology. In particular, the present invention relates to the treatment of cancer.

### BACKGROUND

The tumor microenvironment is infiltrated with diverse innate and adaptive immune cells. These immune cells are under surveillance and control by multiple mechanisms, including signalling suppression. In signalling suppression, the tumor cells downregulate stimulatory immunoreceptors' activity while upregulating the activity of inhibitory immunoreceptors. For example, tumor cells can downregulate T cell receptor (TCR)-mediated stimulatory signalling by reducing surface MHC-I level. On the other hand, tumor cells upregulate PD-1-mediated inhibitory signalling by increasing surface PD-L1 level. Tumor cells evade the control of immune system through manipulation of signalling suppression of the immune cells.

Utilising the same mechanism, therapeutic methods are developed by blocking the activation of inhibitory immunoreceptors and eliciting the antitumor function of immune cells. Various inhibitory immunoreceptors have been identified in past decades for the purpose of treating cancer, for example, programmed cell death-1 (PD-1), cytotoxic T lymphocyte-associated protein-4 (CTLA-4), Lymphocyte-activation gene 3 (LAG3), T cell immunoglobulin domain and mucin domain 3 (TIM3), T cell immunoreceptor with immunoglobulin and ITIM domain (TIGIT) and B- and T-lymphocyte attenuator (BTLA). They are named as “immune checkpoints” referring to molecules that act as gatekeepers of immune responses. Immune checkpoint blockade (ICB) by antibodies targeting molecules such as PD-1 and CTLA4 are among the most widely used cancer immunotherapies.

Immune checkpoint blockade (ICB) has achieved promising outcomes in various malignancies, including hepatocellular carcinoma (HCC), which remains the sixth-most common cancer and fourth leading cause of cancer mortality worldwide. The use of anti-PD-1 immune checkpoint blockade monotherapy in patients with advanced hepatocellular carcinoma (HCC) produced modest objective response rates (ORR) of 15% or 18.3% in phase III trials for nivolumab and pembrolizumab, respectively. In addition, about 20% of the patients experienced grade 3 or higher treatment-induced immune-related adverse events (irAE). While recently reported combination immunotherapies for hepatocellular carcinoma (HCC) conferred greater objective response rates, immune-related adverse events (irAEs) increased in tandem. For instance, anti-PD-1 combined with anti-CTLA4 for advanced hepatocellular carcinoma (HCC) patients resulted in 31% objective response rates (ORR) and 37% grade 3/4 immune-related adverse events (irAEs), and anti-programmed death-ligand 1 (PD-L1) combined with anti-vascular endothelial growth factor-A (VEGF-A) resulted in 27.3% objective response rates (ORR) and 56.5% grade 3/4 immune-related adverse events (irAEs). Immune-related adverse events (irAEs) can be fatal to some patients, or cause delay or disruption to treatment outcome, commonly manifest as systemic autoimmune conditions.

Therefore, what is needed is a method for predicting the response to treatment and potential immune-related adverse events in liver cancer patients for deciding on the treatment of liver cancer. Furthermore, other desirable features and characteristics will become apparent from the subsequent detailed description and the appended claims, taken in conjunction with the accompanying drawings and this background of the disclosure.

### SUMMARY OF INVENTION

In one aspect, the present disclosure provides a method of treating liver cancer in a subject, comprising detecting an immune cell population that comprises one or more biomarkers selected from the group consisting of CXCR3, CD45RO, CCR7, CD8, HLADR, ITGAX (CD11c), and CD86 in a sample obtained from the subject; and administering a therapeutically effective amount of an immune checkpoint inhibitor to the subject if the immune cell population is detected in the sample.

In another aspect, the present disclosure refers to a method of treating liver cancer in a subject, comprising detecting an immune cell population that comprises one or more biomarkers selected from the group consisting of CXCR3, CD45RO, CCR7, CD8, HLADR, CD86, and CD14 in a sample obtained from the subject; and administering a therapeutically effective amount of an immune checkpoint inhibitor to the subject if the immune cell population is detected in the sample.

In another aspect, the present disclosure provides a kit or panel of biomarkers for evaluating complete or partial response of a subject suffering from liver cancer to a treatment with an immune checkpoint inhibitor, the kit or panel comprising at least one antibody adapted to target one or more biomarkers in a sample obtained from the subject, wherein the one or more biomarker is selected from the group consisting of CXCR3, CD45RO, CCR7, CD8, HLADR, ITGAX (CD11c), and CD86.

In yet another aspect, the present disclosure provides a kit or panel of biomarkers for evaluating one or more treatment-induced immune-related adverse events (irAEs) of a subject suffering from liver cancer to a treatment with an immune checkpoint inhibitor, the kit or panel comprising at least one antibody adapted to target one or more biomarkers in a sample obtained from the subject, wherein the one or more biomarker is selected from the group consisting of CXCR3, CD45RO, CCR7, CD8, HLADR, CD86, and CD14.

### BRIEF DESCRIPTION OF THE DRAWINGS

FIG. 1 provides a schematic overview of the coupling mechanism between response to immune checkpoint blockade and the immune-related adverse events (irAEs) in the context of liver cancer through cell-cell signalling. Immune biomarkers are identified to predict the patients' response and adverse events to treatment with immune checkpoint inhibitors In the present disclosure, a combination immune checkpoint blockade immunotherapy is provided using an exemplary mouse liver cancer model to uncouple the response and immune-related adverse events (irAEs), resulting in reduction in both tumor load and adverse effects from the treatment.

FIG. 2A provides a schematic summary of the clinical study design and workflow. The Singaporean cohort is used as the discovery cohort. Pre- and on-treatment blood samples from hepatocellular carcinoma (HCC) patients receiving treatment with anti-PD-1 immune checkpoint inhibitors. The Singaporean (SG) cohort is analysed using Cytometry by Time Of Flight (CyTOF) and single cell RNA sequencing (scRNA-seq) to uncover the mechanism of response and immune-related adverse events (irAEs). An independent Korean cohort (KR) is used as a validation cohort and analysed using flow cytometry for defined biomarkers identified from the SG cohort. Further validation is conducted by bulk RNA sequence analysis of pre- and 1-week on-treatment tumour biopsies (SG cohort) and using a murine hepatocellular carcinoma (HCC) model. Based on the treatment outcome, the patients are stratified as: Responders (Res), and Non-responders (Non-Res). Using patents' sample from two independent cohorts and an in vivo murine model for hepatocellular carcinoma (HCC), biomarkers useful for prediction of response and immune-related adverse events (irAEs) are identified.

FIG. 2B provides a comprehensive CyTOF analysis that reveals clusters corresponding to major immune lineages and subtypes according to the relative expression of 38 immune markers. Single-cell mass cytometry by time-of-flight (CyTOF) analysis of peripheral immune cells from anti-PD-1 treated hepatocellular carcinoma (HCC) patients. The t-distributed Stochastic Neighbor Embedding (tSNE) plot shows 100 immune clusters from unsupervised down-dimensioning and cell clustering using FlowSOM. The CyTOF analysis provides a comprehensive profile of the surface markers of the immune cells obtained from the patients.

FIG. 2C shows a heatmap summarising the normalised relative expression of all 38 markers expressed by the 100 immune clusters.

FIG. 2D demonstrates the identification of immune clusters enriched in responders (Res) or non-responders (Non-Res) patients. FIG. 2D provides a heatmap showing scaled median expression of selected key markers identified in the immune clusters enriched in the responders (Res) or non-responders patient groups.

FIG. 2E shows a t-distributed Stochastic Neighbor Embedding (tSNE) plot presenting enriched immune clusters (“C”) identified in responders (Res) or non-responders (Non-Res). An initial unsupervised Mann-Whitney analysis of six responders (Res) versus six non-responders (Non-Res) clinically matched sample revealed two CD4⁺ clusters: FoxP3⁺CD4⁺ T cells (C33) and FoxP3⁺CTLA4⁺CD4⁺ regulatory T cells (Treg) (C3), and a CD8⁺CD45RO⁺CCR7⁻CXCR3⁺ T_{EM} (C76) cluster that are enriched in responders (Res) group. The tSNE plots provide visualisation of distinctive cell clusters identified according to the surface marker profiling.

FIG. 2F shows box plots showing the enrichment trends (Mann-Whitney U test with two-tailed p<0.1) of each unsupervised immune cluster (C) in either responders (Res) (n=6) or non-responders (Non-Res) (n=6) groups of the SG cohort indicated by unsupervised CyTOF analysis pipeline. Box plots show median and interquartile range. ** denote two-tailed p<0.01 by unpaired Mann-Whitney U test. Baseline samples are indicated as black circles. The abundance of live immune cells in responder (Res) and non-responder (Non-Res) groups are shown for each cell cluster identified. Clusters C33, C3, C76, and C4 show enrichment in responder group while cluster C37 preferably enriches in Non-Res group.

FIG. 2G shows the immune subsets analyses for response (Res) vs non-response (Non-Res) groups of SG cohort treated with anti-PD-1 by supervised manual gating with FlowJo. Representative dot plots showing manual gating strategy for enriched immune cell populations from the CyTOF data.

FIG. 2H presents box plots showing manually-gated immune subsets identified in responders (Res) (n=8) or non-responders (Non-Res) (n=13) from the SG cohort based on FIG. 2F. Median and interquartile range shown. Baseline samples indicated as black circles. *, ** denote two-tailed P<0.05 and P<0.01 by unpaired MWU test. These results confirm the significant enrichment of the immune subsets of Tregs (C3), CXCR3⁺CD8⁺ T_{EM} cells (C76) and APCs (C4) in responders (Res) and the enrichment of myeloid-derived suppressor cells (MDSCs) in non-responders (Non-Res).

FIG. 2I provides box plots showing median and interquartile range of the data distribution. * denotes two-tailed p<0.05 and NS denotes non-significant respectively by unpaired Mann-Whitney U test. The clusters of Tregs, CXCR3⁺CD8⁺ T_{EM} cells, APCs, and myeloid-derived suppressor cells (MDSCs) show similar enrichment frequencies in pre- or early on-treatment (<6 weeks) blood, particularly in the responders (Res). Thus, the identification of these clusters is independent from the whether a treatment is provided or not to the patients.

FIG. 2J provides further validation on the enrichment of peripheral Tregs, CXCR3⁺CD8⁺ T_{EM} cells and APCs in responders (Res), and MDSCs in non-responders (Non-Res) by flow cytometric analysis of an independent anti-PD1-treated KR cohort (n=29). Flow cytometric analyses for response (Res) vs non-response (Non-Res) in the independent Korea cohort of anti-PD-1 treated hepatocellular carcinoma (HCC) patients is provided. Representative dot plots showing gating strategy for enriched immune cell populations from the flow cytometry data.

FIG. 2K shows box plots summarising manually-gated immune subsets in responders (Res) (n=9) or non-responders (Non-Res) (n=20) from the KR cohort. Median and interquartile range shown. Baseline samples indicated as black circles. *, ** denote two-tailed P<0.05 and P<0.01 by unpaired MWU test. Results in the KR cohort validated the enrichment of Tregs, CXCR3⁺CD8⁺ T_{E}M cells and APCs in responders (Res), and MDSCs in non-responders (Non-Res), similar to what was identified in the SG cohort.

FIG. 2L shows Kaplan-Meier graphs providing progression-free survival (PFS) profiles of SG (n=21) and KR cohorts (n=29). Log-rank test P-values are shown. The Kaplan-Meier analyses showed that higher frequencies of Tregs, APCs and CXCR3⁺CD8⁺ T_{EM} cells are significantly associated with superior progression-free survival (PFS) in both cohorts.

FIG. 2M investigates the association between the immune-related adverse events (irAEs) status of the patients and the immune biomarkers identified with response. Box plots show median and interquartile range of the frequencies of the identified cell subsets. * denotes two-tailed p<0.05 and NS denotes non-significant respectively by unpaired Mann-Whitney U test. The patients are segregated according to their immune-related adverse events (irAEs) status into toxicity (Tox) or non-toxicity (Non-Tox) groups. CXCR3⁺CD8⁺ T_{EM} cells and APCs remain significantly enriched in Res, particularly in Non-Tox patients, from both SG and KR independent cohorts.

FIG. 2N shows box plots presenting the enrichment trends of the identified immune subsets according to 4-grouping analysis between Res/Tox (n=3), Non-Res/Tox (n=1), Res/Non-Tox (n=6) and Non-Res/Non-Tox (n=19). Box plots show median and interquartile range. **, * and NS denote two-tailed p<0.01, p<0.05 and non-significant respectively by unpaired Mann-Whitney U test. Tregs, CXCR3⁺CD8⁺ T_{EM} cells and APCs show significant enrichment in Responders (Res) group, particularly in Non-Tox patients, from both SG and KR independent cohorts. Thus, based on the results in FIG. 2L to FIG. 2N, peripheral CXCR3⁺CD8⁺ T_{EM} and APCs are identified as independent predictors of response and progression-free survival (PFS) in hepatocellular carcinoma (HCC) patients treated with anti-PD-1 immune checkpoint blockade.

FIG. 3A shows a heatmap summarising the scaled median expression of key markers in the immune clusters enriched in either toxicity (Tox) group (≥Grade 2 irAEs) or non-toxicity (Non-Tox) group (Grade 1 or no irAEs). Blood samples are obtained from patients during or close to (±2-weeks)≥Grade 2 immune-related adverse events (irAEs) for toxicity (Tox) group and patients of matched post-immune checkpoint blockade timepoints from non-toxicity (Non-Tox) group who developed no or Grade 1 irAEs. Due to differences in the study design, this analysis is only performed for the SG cohort.

FIG. 3B shows in t-distributed Stochastic Neighbor Embedding (tSNE) plots enriched immune clusters (“C”) in Tox or Non-Tox groups. Two CXCR3⁺CD38⁺CD16⁺CD56⁺ NK clusters (C89 and 99) show enrichment in toxicity (Tox) group. Conversely, three CD8⁺ clusters (C66, 76 and 96) including C66 and C76 T_{EM} (CD45RO⁺CCR7⁻) cells and C96 (Vα7.2⁺CD161⁺CD56⁺CD8⁺) mucosal-associated invariant T (MAIT) cells as well as a CD11c⁺CD14⁺HLADR⁺ myeloid cluster (C27), showed enrichment in Non-Tox group.

FIG. 3C provides detailed analyses for immune subsets related to immune-related adverse events (irAEs) from SG cohort hepatocellular carcinoma (HCC) patients treated with anti-PD-1 immune checkpoint blockade. Box plots shows enrichment trends (Mann-Whitney U test with two-tailed p<0.1) of immune clusters (C) in Tox (n=8) vs Non-Tox (n=11) groups using unsupervised analysis. Box plots show median and interquartile range. * and NS denote two-tailed p<0.05 and non-significant respectively by unpaired Mann-Whitney U test. Samples are limited to those ±2 weeks from ≥Grade 2 irAEs manifestation (Tox) vs those with G1 irAEs or without irAEs (Non-Tox) at matched time point.

FIG. 3D shows representative dot plots presenting the manual gating strategy for enriched immune cell populations from the Cytometry by Time Of Flight (CyTOF) results.

FIG. 3E provides box plots summarising the manually-gated immune subsets in Tox (n=9) or Non-Tox (n=11) groups. Median and interquartile range shown. *, ** denote two-tailed P<0.05 and P<0.01 by unpaired MWU test. The results from FIG. 3D and FIG. 3E confirmed the enrichment of CXCR3⁺CD38⁺CD16⁺CD56⁺ NK clusters (C89 and 99) in toxicity (Tox) group, and the enrichment of three CD8⁺ clusters (C66, 76 and 96) including C66 and C76 T_{FM} (CD45RO⁺CCR7⁻) cells, C96 (Vα7.2⁺CD161⁺CD56⁺CD8⁺) mucosal-associated invariant T (MAIT) cells, and a CD11c⁺CD14⁺HLADR⁺ myeloid cluster (C27) in Non-Tox group.

FIG. 3F provides box plots showing the enrichment trends (Mann-Whitney U test with two-tailed p<0.1) of five identified immune subsets between Tox/Res (n=6), Non-Tox/Res (n=6), Tox/Non-Res (n=3) and Non-Tox/Non-Res (n=5). Box plots show median and interquartile range. * and NS denote two-tailed p<0.05 and non-significant respectively by unpaired Mann-Whitney U test. Samples are limited to those ±2 weeks from ≥Grade 2 irAEs manifestation (Tox) vs those with Grade 1 or without irAEs (Non-Tox) at matched time point. All five immune subsets displayed similar trends with or without a response to anti-PD-1 treatment, indicating independence of the immune-related adverse events (irAEs) to response to treatment in patients.

FIG. 4A summarises a comprehensive scRNA-seq analysis of immune subsets associated with response and immune-related adverse events (irAEs) to investigate molecular and mechanistic insights of on-treatment transcriptomic perturbations in the immune subsets identified. The scRNA-seq is conducted on 10 peripheral blood mononuclear cell (PBMC) samples consisting of nine on-treatment PBMCs (6 Res versus 3 Non-Res; 5 Tox versus 4 Non-Tox) and one matched pre-treatment sample (Res/Tox). The t-distributed Stochastic Neighbor Embedding (tSNE) plot shows 29 scRNA-seq clusters.

FIG. 4B provides a heatmap illustrating the top 10% of the differentially-enriched genes (DEGs) of the 29 clusters identified from the scRNA-seq of FIG. 4A. Abbreviations of the terms in FIG. 4B include: Eff—Effector; Imm—Immature; Prolif—Proliferative; Th2—T helper 2 cells; cDC—Conventional dendritic cells; and pDCs—Plasmacytoid DCs. These genes identified are highly differentially expressed among the 29 clusters of cells, indicating their potential function/effects specific to these clusters related to the treatment induced response and adverse events.

FIG. 4C provides box plots showing cell clusters enriched in Res (n=6) or Non-Res (n=3) and Tox (n=5) or Non-Tox (n=4). Treg (CD3D⁺CD4⁺FOXP3⁺CTLA4⁺IL2RA⁺) and an APC cluster cDC1 expressing ITGAX (CD11c), HLA-DPA1, THBD (CD141), and CLEC9A are found significantly enriched in Res group compared to the Non-Res group. Median and interquartile range shown. * denotes two-tailed P<0.05 by unpaired MWU test.

FIG. 4D provides box plots showing cell clusters enriched in Res (n=6) or Non-Res (n=3) and Tox (n=5) or Non-Tox (n=4). Two CD14 and ITGAX (CD11c)-expressing myeloid clusters, CD14-1 and CD14-3, are found associated with Non-Tox group compared to Tox group. Median and interquartile range shown. * denotes two-tailed P<0.05 by unpaired MWU test.

FIG. 4E summarises the scRNA seq analyses of immune subsets of interest, including Treg, cDC1, CD14-1, and CD14-3. Heatmap showing the top 20 enriched genes in the five enriched immune cell clusters either in response or irAEs. Clusters were down-sampled to a maximum of 300 cells for plotting. Arrows and boxes highlight genes of interest.

FIG. 4F provides two tSNE plots showing enrichment of cell clusters Treg and cDC1 in Res (n=6) compared to Non-Res (n=3).

FIG. 4G provides two tSNE plots showing enrichment of cell clusters CD14-1 and CD14-3 in Tox (n=5) compared to Non-Tox (n=4).

FIG. 4H provides a collection of 13 violin plots indicating the expression levels of selected DEGs from the four enriched immune subsets identified for response and irAEs to decipher the immune mechanisms behind the distinct clinical fates. The cDC1 cluster enriched in Res expressed the highest level of HLA genes, suggesting superior antigen presentation capability. Comparison of the myeloid clusters (CD14-1 and CD14-3) associated with non-Tox group reveals that CD14-1 expresses higher levels of antigen presenting HLA-related genes than CD14-3. Conversely, CD14-3 expressed higher levels of immunosuppressive STAB1 (stabilin-1).

FIG. 4I shows top 10 significant pathways by fold-enrichment for cDC1. Vertical axis represents −log₁₀ (Benjamini)-adjusted P-value and colour gradient represents fold-enrichment of each pathway. In agreement with the high HLA genes expression in cDC1 cluster shown in FIG. 4H, cDC1 shows enrichment in antigen processing and presentation pathways via MHC class II, T cell co-stimulation, and interferon-gamma-mediated signalling, which are important for immune priming.

FIG. 4J shows top 5 significant pathways by fold-enrichment for both CD14-1 and CD14-3. Vertical axis represents −log₁₀ (Benjamini)-adjusted P-value and colour gradient represents fold-enrichment of each pathway. Among the enriched functional pathways, peptide antigen assembly with MHC class II and the pro-inflammatory interleukin-1 beta pathway are enriched in CD14-1 but not in CD14-3. Thus, among these CD14 clusters, CD14-3, which is more significantly associated with Non-Tox group according to FIG. 4D, displays reduced antigen presentation/inflammatory characteristics and a more immunosuppressive phenotype than CD14-1.

FIG. 5A provides scRNA-seq data illustrating the strategy to segregate CXCR3⁺CD8⁺ T cells from all CD8+ T cells based the expression profiles of CD3D, CD8A and CXCR3. CXCR3⁺CD8⁺ T cells are filtered based on expression level threshold>0.5, giving a total of 863 single cells, as depicted in the enlarged box below.

FIG. 5B shows that CXCR3⁺CD8⁺ T_{EM} cells are involved in both response and immune-related adverse events (irAEs). Volcano plot demonstrates differentially-expressed genes (DEGs) comparing CXCR3⁺CD8⁺ T_{EM} cells against all T cells. Compared to other T cells, multiple genes involved in antigen presentation, HLA(s), inflammation, granzymes (GZM)s and proliferation (MKI67) are enriched in the CXCR3⁺CD8⁺ T cells. Conversely, expression of naïve T cell markers like CCR7, IL7R and LEF1 are downregulated, suggesting an effector memory phenotype. Selected genes are highlighted in boxes as upregulated (Up): P<0.05 & ln(FC)>0.25, downregulated (Down): P<0.05 & ln(FC)<−0.25 or Non-significant (NS): ln(FC)<±0.25.

FIG. 5C shows top 10 significantly-enriched functional pathways from DEGs in CXCR3⁺CD8⁺ T_{EM} cells. Vertical axis represents the −log₁₀(Benjamini) adjusted P-value and colour gradient represents fold-enrichment. Enriched functional pathways include inflammatory response, cytolysis and antigen processing and presentation via MHC class II, in agreement with the findings in FIG. 5A. Thus, these CXCR3⁺CD8⁺ T_{EM} cells display a more inflammatory and cytolytic phenotype compared to other T cells.

FIG. 5D provides dot plots of selected ligand-receptor interacting pairs between CXCR3+CD8+ T_{EM} cells and all other immune clusters computed using CellPhoneDB to identify the expression of receptors and ligands in CXCR3⁺CD8⁺ T_{EM} cells and predict their potential cell-cell communications with other immune cells in Res/Non-Res groups. Point shade reflects log 2Mean of average expression levels of interacting molecule 1 from cluster 1 and interacting molecule 2 from cluster 2. Point size indicates the −log₁₀(P-value).

FIG. 5E provides dot plots of selected ligand-receptor interacting pairs between CXCR3⁺CD8⁺ T_{EM} cells and all other immune clusters computed using CellPhoneDB to identify the expression of receptors and ligands in CXCR3⁺CD8⁺ T_{EM} cells and predict their potential cell-cell communications with other immune cells in Tox/Non-Tox groups. Point shade reflects log 2Mean of average expression levels of interacting molecule 1 from cluster 1 and interacting molecule 2 from cluster 2. Point size indicates the −log₁₀(P-value). Lymphotoxin alpha (LTA) and its receptors, tumour necrosis factor receptor superfamily (TNFRSF) 1A, 1B and lymphotoxin beta receptor (LTBR), which promotes inflammation and oncogenesis, are enriched in both Res and Tox groups. This suggests that CXCR3⁺CD8⁺ T_{EM} cells form pro-inflammatory interactions with other cells, leading to both response and immune-related adverse events (irAEs). Distinct tumour necrosis factor (TNF) interactions between CXCR3⁺CD8⁺ T_{EM} and myeloid cell populations are observed in FIG. 5C and FIG. 5D, where TNF-TNFRSF1B (TNFR2) is enriched in Res group, but TNF-TNFRSF1A (TNFR1) is enriched in Non-Tox group instead.

FIG. 5F shows flow cytometric results validating the TNF/TNFR ligand/receptors expression. The interactions of TNF with TNFRSF1A and 1B play important roles in macrophage activation and inflammation. To validate the protein expression of TNFα, TNFR1 and TNFR2, flow cytometry is performed on peripheral blood mononuclear cells (PBMCs) from immune checkpoint blockade-treated hepatocellular carcinoma (HCC) patients. Representative dot plots show manual gating for the key immune subsets, TNFα, TNFR1 and TNFR2 on post-treatment PBMCs. For TNFα staining, 6h PMA/Ionomycin stimulation is used.

FIG. 5G provides box plots showing expression level of TNFα for the key immune subsets from Res (n=11), Non-Res (n=5), Tox (n=8) or Non-Tox (n=8) in patient peripheral blood mononuclear cells (PBMCs). Only two-tailed *, **p<0.05 or p<0.01 respectively by unpaired Mann-Whitney U test are reported. Consistent with the data shown in FIG. 5C, CXCR3⁺CD8⁺ T_{EM} cells express significantly higher TNFα in Res group compared to Non-Res group.

FIG. 5H provides box plots showing expression level of TNFR1 for the key immune subsets from Res (n=11), Non-Res (n=5), Tox (n=8) or Non-Tox (n=8) in patient peripheral blood mononuclear cells (PBMCs). Only two-tailed *, **p<0.05 or p<0.01 respectively by unpaired Mann-Whitney U test are reported. Increased expression of TNFR1 on both CD14⁺ monocytes and CD14⁻CD11c⁺HLA⁻ DR⁺ DC is observed in Non-Tox group compared to the Tox group.

FIG. 5I provides box plots showing expression level of TNFR2 for the key immune subsets from Res (n=11), Non-Res (n=5), Tox (n=8) or Non-Tox (n=8) in patient peripheral blood mononuclear cells (PBMCs). Only two-tailed *, **p<0.05 or p<0.01 respectively by unpaired Mann-Whitney U test are reported. There is no significant difference observed in TNFR2 expression in monocytes and DCs between Res and Non-Res groups, indicating that the increased TNF interaction in Res (FIG. 5D) is largely driven by TNFα upregulation, while in Non-Tox group (FIG. 5E), it is primarily due to increased TNFR1 expression. This suggests that different TNF signalling pathways are harnessed to uncouple response and immune-related adverse events (irAEs) in immune checkpoint blockade.

FIG. 6A shows differences in frequencies of immune subsets from responders' (Res) matched peripheral blood mononuclear cells (PBMCs) taken from patients at early, <6 weeks (6W) versus late, >10 weeks (10 W) time points after anti-PD-1 treatment (n=7); * denotes P<0.05; NS denotes non-significant two-tailed P-values by Wilcoxon signed-rank test. Comparing to frequency of the response-associated immune subsets (FIG. 2H and FIG. 2K), a significant reduction in APCs and CXCR3⁺CD8⁺ T_{M} cells in late (>10 weeks) on-therapy blood samples is found compared to the matched early (<6 weeks) blood samples in Responder (Res) group. The changes of the frequencies of these subgroups reflects the trafficking of immune cells from the blood into tumour tissue.

FIG. 6B shows tissues recruitment analysis for non-responders (Non-Res). Matched analysis of immune subsets from the non-responders' (Non-Res) blood samples taken from early, <6 weeks (6 W) versus late, >10 weeks (10 W) after anti-PD-1 treatment. N=2, paired statistical analyses not applicable. The previously observed reduction in frequencies of APCs and CXCR3⁺CD8⁺ T_{EM} cells in late (>10 weeks) on-therapy blood samples compared to the matched early (<6 weeks) samples (FIG. 6A) is not found in the Non-Res group.

FIG. 6C provides a heatmap summarising the results from a bulk tissue RNA-sequence on pre- and 1 week on-treatment tumour biopsies from 10 immune checkpoint blockade-treated hepatocellular carcinoma (HCC) patients (6 Res, 4 Non-Res). Differentially-enriched genes (DEGs) comparing pre- versus on-treatment matched tumour tissues in responders (Res) to immunotherapy (n=6) are shown in the heatmap with selected differentially-enriched genes (DEGs) of interest highlighted. Differentially-enriched genes (DEGs) analysis comparing on- versus pre-treatment tumours from Res group reveals upregulation of genes related to T cell activation (GZMA, GZMH) and antigen presentation (HLA-related genes) upon treatment. Notably, the same genes are also upregulated in CXCR3⁺CD8⁺ T_{EM} cells and APCs.

FIG. 6D shows top 10 significantly-enriched functional pathways of upregulated differentially-enriched genes (DEGs) in matched pre-treatment versus on-treatment tumour tissues from the responders (Res) group. Horizontal-axis represents the −log₁₀(Benjamini) adjusted p-value and shading gradient represents enrichment fold. On-treatment enriched functional pathways from the responders (Res) include antigen presentation, T cell co-stimulation, leukocyte chemotaxis, and IFNγ-mediated signalling. many of which are common functional pathways enriched in both cDC1 and CXCR3⁺CD8⁺ T_{EM} cells, suggesting that these immune cells are recruited to the tumour tissue following immune checkpoint blockade, particularly in responders (Res). Moreover, the enrichment of the key chemokines that bind to CXCR3 including CXCL9, CXCL10 and CXCL11 in responders (Res), further supports tumour recruitment of CXCR3⁺CD8⁺ T_{EM} cells in responders (Res).

FIG. 6E provides a heatmap summarising all up- (26) and down-regulated (9) genes in on-treatment (1 week) compared to pre-treatment matched tumour tissues in non-responders (Non-Res) to immunotherapy (n=4). Genes were selected based on the cut-offs of padj-value<0.05 and log₂(fold-change)>0.5 or <−0.5. Unlike the results from FIG. 6D, the non-responders (Non-Res) show a different set of differentially-enriched genes (DEGs) unrelated to immune activation.

FIG. 6F provides analysis of five immune subsets from matched peripheral blood mononuclear cells (PBMCs) taken before (Pre-Tox) and at the point of immune-related adverse events (irAEs) manifestation (Tox) after anti-PD-1 treatment (n=6); * and NS denotes P<0.05 or non-significant two-tailed P-values by Wilcoxon signed-rank test. As shown in FIG. 6F, CXCR3⁺CD8⁺ T_{EM} cells are significantly depleted from the blood at the point of irAEs manifestation, suggesting their recruitment to the tissue. This result highlights the importance of CXCR3-mediated migration of CXCR3⁺CD8⁺ T_{EM} cells in the manifestation of response and immune-related adverse events (irAEs).

FIG. 7A provides a schematic summary of the combination immunotherapies of anti-TNFR1 or anti-TNFR2 with anti-PD-1 in a murine hepatocellular carcinoma (HCC) model. Mice with hepatocellular carcinoma (HCC) induced by hydrodynamic tail-vein injection of Hepa1-6 cells are randomly assigned to six treatment groups and treated on day 7, day 11, day 14 and day 18 before the tumours were harvested for analysis on day 21 (n=5 mice per group).

FIG. 7B shows representative images of livers harvested from the mice at Day−21 of combination immunotherapies experiment. Tumor modules are visible in the harvested livers for most of the tested groups except for anti-PD-1+ anti-TNFR2.

FIG. 7C provides quantification of tumour nodules from the mice undergoing combination immunotherapies experiment. At harvest on Day−21, all mice receiving combination treatments showed significant reduction in tumour nodules, especially those treated with anti-PD-1+anti-TNFR2, which displayed no tumour burden. NIL, no tumours from all mice treated with anti-PD-1+anti-TNFR2. Box plots show median and interquartile range. *, **, NS denote two-tailed P<0.05, P<0.01 or non-significant two-tailed P-values by unpaired MWU test.

FIG. 7D shows liver/body weight ratio of the mice from each condition. Box plots show median and interquartile range. *, **, NS denote two-tailed P<0.05, P<0.01 or non-significant two-tailed P-values by unpaired MWU test. A significantly higher liver-to-body weight ratio is observed in the mice treated with the anti-PD-1+anti-TNFR1 combination, but not in any other groups.

FIG. 7E provides the measurement of mice body weight (g) at harvest Day 21 showing non-significant differences across treatment groups (n=5 mice per group) in body weights. Despite comparable body weights, the significantly higher liver-to-body weight ratio in mice treated with the anti-PD-1+anti-TNFR1 combination (FIG. 7D) suggests liver hypertrophy and inflammation.

FIG. 7F provides an analysis on frequencies (%) of CD8 T cells and CD69⁺ active CD8 T cells in non-tumour liver tissues of the mice. Box plots show median and interquartile range. *, **, NS denote two-tailed P<0.05, P<0.01 or non-significant two-tailed P-values by unpaired MWU test. Anti-PD1+anti-TNFR1 combination group shows increased frequencies (%) of both CD8 T cells and CD69⁺ active CD8 T cells in non-tumour liver tissues. Based on previous observation, higher TNFR1 expression is shown in Non-Tox group (FIG. 5E and FIG. 5H), indicating its role in preventing immune-related adverse events (irAEs). Such observation corroborates the enhanced toxicity observed in mice treated with anti-PD1+anti-TNFR1 combination.

FIG. 7G provides representative dot plots showing manual gating strategy of flow cytometry data for enriched immune cell populations from tumour and non-tumour liver tissues from hepatocellular carcinoma (HCC) murine model. This observation further supports increasing CD8⁺ T cells infiltration, especially the pro-inflammatory CD69⁺ activated CD8⁺ T cells, in the non-tumour liver tissue.

FIG. 7H shows representative immunohistochemistry images of CD4 and DAPI on FFPE colon sections from mice across treatment groups. Bar=50 mm. The images show enhanced colonic CD4+ T cell infiltration, indicating colitis and intestinal inflammation in the mice.

FIG. 7I provides box plots showing enrichment (Mann-Whitney U test with two-tailed *p<0.05) of colon-infiltrating CD4 T cells in anti-PD-1+anti-TNFR1 combination treatment vs Anti-PD1 and isotype antibodies control groups (n=5 mice per group). Enhanced colonic CD4⁺ T cell infiltration indicates colitis and intestinal inflammation. These observations support that higher TNFR1 expression prevents immune-related adverse events (irAEs), corroborates the enhanced toxicity observed in mice treated with anti-PD1+anti-TNFR1 combination. Enhanced toxicity are not observed in the anti-PD-1+anti-TNFR2 combination, which displayed the highest tumour control, further strengthening the hypothesis that differential blockade of TNFR1 or TNFR2 combined with anti-PD-1 therapy uncouples response and immune-related adverse events (irAEs).

FIG. 7J shows representative dot plots for cell sorting strategy of CD3⁺CD4⁺CD25⁺CD127⁻ Treg cells and CD3⁺CD4⁺CD25⁻CD127⁺ non-Treg, and RNA sequencing of TNFRSF1A and TNFRSF1B from peripheral blood mononuclear cells (PBMCs) and tumours of hepatocellular carcinoma (HCC) patients. The selective enhanced response following TNFR2 inhibition could stem from the preferential expression of TNFR2 on highly immunosuppressive Tregs. To validate this, Tregs and non-Tregs from PBMCs, adjacent non-tumour liver and tumour tissues from hepatocellular carcinoma (HCC) patients are sorted.

FIG. 7K provides box plots showing the enrichment of TNFRSF1B (TNFR2), but not TNFRSF1A (TNFR1) on Tregs versus non-Treg cells from tumour-infiltrating leukocytes (TILs) versus peripheral blood mononuclear cells (PBMCs) and non-tumour liver-infiltrating leukocytes (NILs) of hepatocellular carcinoma (HCC) patients (n=4), p values*two-tailed p<0.05 from paired Wilcoxon test. A significantly higher expression of TNFRSF1B (TNFR2), but not TNFRS1A (TNFR1) is identified in Tregs compared to non-Tregs in tumour-infiltrating leukocytes (TILs). TNFRSF1B expression is also higher in Tregs from TILs compared to Tregs from PBMCs or non-tumour liver-infiltrating leukocytes. These findings demonstrated the specificity of TNFR2 expression on Tregs from hepatocellular carcinoma (HCC) tumours, which upon selective inhibition, could enhance anti-tumour response but not systemic toxicity.

FIG. 7L shows frequencies (%) of CXCR3⁺CD8⁺ T cells and CD11c⁺MHCII⁺XCR1⁺cDC1 cells in tumour tissues of the mice. NA, no tumours from anti-PD-1+anti-TNFR2 treatment group. Box plots show median and interquartile range. *, **, NS denote two-tailed P<0.05, P<0.01 or non-significant two-tailed P-values by unpaired MWU test. Intratumoral enrichment of CXCR3⁺CD8⁺ T cells and CD11c⁺MHCII⁺XCR1⁺ cDC1 is observed in the mice treated with anti-PD-1, which is further enhanced by the anti-PD-1+anti-TNFR1 combination that corresponds to enhanced tumour control, suggesting recruitment of these cells to tumours in responders (Res).

FIG. 7M shows frequencies (%) of CXCR3⁺CD8⁺ T cells and CD11c⁺MHCII⁺XCR1⁺cDC1 cells in non-tumour liver tissues from the mice. Box plots show median and interquartile range. *, **, NS denote two-tailed P<0.05, P<0.01 or non-significant two-tailed P-values by unpaired MWU test. Notably, the anti-PD-1+anti-TNFR1 combination group which displays enhanced immune-related adverse events (irAEs), also displays a significantly higher infiltration of CXCR3⁺CD8⁺ T cells in the non-tumour liver tissue, validating recruitment of these cells to irAE sites.

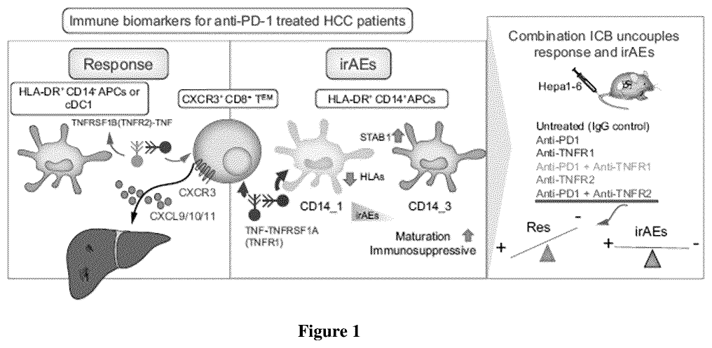

### DEFINITIONS

As used herein, the term “immune checkpoint protein” refers to the regulators of immune activation which play a key role in maintaining immune homeostasis and preventing the immune system from attacking cells indiscriminately. They are named as “immune checkpoints proteins” because these molecules act as gatekeepers of immune responses. Immune checkpoint molecules involve both costimulatory and inhibitory proteins. Costimulatory proteins can promote cell survival, cell cycle progression and differentiation to effector and memory cells, whereas inhibitory proteins terminate these processes to halt ongoing inflammation. Examples of immune checkpoint proteins include programme cell death 1 (PD-1), programme cell death ligand 1 (PD-L1), cytotoxic T lymphocyte-associated protein 4 (CTLA-4), TIGIT, LAG3, and Tim3.

As used herein, the terms “immune checkpoint blockade (ICB)”, “immune checkpoint blockade therapy”, “ICB therapy”, “immune checkpoint blockade treatment” or “ICB treatment” refer to the treatment of cancer using immune checkpoint inhibitors. Examples of immune checkpoint inhibitors include, but are not limited to, anti-programme cell death 1 (anti-PD-1), anti-programme cell death ligand 1 (anti-PD-L1), anti-cytotoxic T lymphocyte-associated protein 4 (anti-CTLA-4), anti-TIGIT, anti-LAG3, and anti-Tim3.

The term “programmed cell death 1 (PD-1)” refers to an immune checkpoint protein, which is an immunoinhibitory receptor belonging to the CD28 family. PD-1 is expressed predominantly on previously activated T cells in vivo, and binds to two ligands, PD-L1 and PD-L2. Immune checkpoint blockade (ICB) by antibodies targeting PD-1 is among the most widely used cancer immunotherapy. As used herein, anti-PD-1 or PD1 inhibitors refers to a monoclonal antibody used for ICB treatment, and includes, for example, but are not limited to, nivolumab, ipilimumab and pembrolizumab.

The term “programmed death-ligand 1 (PD-L1)” refers to one of two cell surface glycoprotein ligands for PD-1 (the other being PD-L2) that downregulate T cell activation and cytokine secretion upon binding to PD-1. As used herein, anti-PD-L1 or PD-L1 inhibitor refers to a monoclonal antibody used for ICB treatment, and includes, for example, but not limited to, atezolizumab, avelumab, and durvalumab.

The term “cytotoxic T lymphocyte-associated protein 4 (CTLA-4)” refers to an immune checkpoint protein, which is an immunoinhibitory receptor belonging to the CD28 family. CTLA-4 is expressed exclusively on T cells in vivo, and binds to two ligands, CD80 and CD86. As used herein, anti-CTLA-4 or CTLA inhibitor refers to an inhibitor used for immune checkpoint blockade treatment. In some examples, anti-CTLA-4 refers to a monoclonal antibody of CTLA-4, and includes, for example but is not limited to, ipilimumab (ATC code L01FX04). The terms “TIGIT”, “T cell immunoreceptor with Ig and ITIM domains”, “WUCAM”, or “Vstm3” refer to an immune receptor present on activated T cells, regulatory T cells, and natural killer cells (NK). TIGIT binds to two ligands, CD155 (PVR) and CD112 (PVRL2, nectin-2), that are expressed by tumor cells and antigen-presenting cells in the tumor microenvironment. TIGIT has been shown to regulate T cell-mediated and natural killer cell-mediated tumor recognition in vivo and in vitro. As used herein, anti-TIGIT or TIGIT inhibitor refers to an inhibitor used for immune checkpoint blockade treatment. In some examples, anti-TIGIT refers to a monoclonal antibody specifically targeting TIGIT, and includes, for example but is not limited to, BMS-986207.

The term “LAG3” or “Lymphocyte Activating 3” refers to a member of the immunoglobulin superfamily that binds to MHC class II (MHCII), FGL-1, α-synuclein fibrils (α-syn), the lectins galectin-3 (Gal-3) and lymph node sinusoidal endothelial cell C-type lectin (LSECtin). LAG3 is an immune checkpoint protein with relevance in cancer, infectious disease and autoimmunity. In particular, LAG3 inhibits the activation of its host cell and generally promotes a more suppressive immune response. For example, on T cells, LAG3 reduces cytokine and granzyme production and proliferation while encouraging differentiation into T regulatory cells. As used herein, anti-LAG3 or LAG3 inhibitor refers to an inhibitor used for immune checkpoint blockade treatment. In some examples, anti-LAG3 refers to a monoclonal antibody specifically targeting LAG3, and includes, for example but is not limited to, TSR-033.

The term “TIM3” or “T cell immunoglobulin domain and mucin domain 3” refers to a member of the TIM family and is originally identified as a receptor expressed on interferon-γ-producing CD4⁺ and CD8⁺ T cells. TIM3 is part of a module that contains multiple co-inhibitory receptors (checkpoint receptors), which are co-expressed and co-regulated on dysfunctional or ‘exhausted’ T cells in chronic viral infections and cancer. As used herein, the anti-TIM3 or TIM3 inhibitor refers to an inhibitor used for immune checkpoint blockade treatment. In some examples, anti-TIM3 refers to a monoclonal antibody specifically targeting TIM3, and includes, for example but is not limited to, LY3321367 and Sym023.

As used herein, the term “liver cancer” refers to malignant tumour or cancer that forms in the tissue of the liver. Examples of liver cancer include, but are not limited to, hepatocellular carcinoma, cholangiocarcinoma, and hepatoblastoma. Hepatocellular carcinoma is a type of adenocarcinoma and the most common type of liver tumour. Cholangiocarcinoma or bile duct cancer is a rare disease in which malignant cancer cells form in the bile ducts. Heptatoblastoma are a type of liver tumour that occurs in infant and children.

As used herein, the term “objective response rate (ORR)” refers to the percentage of subjects in a study or treatment group who have a partial response or complete response to the treatment as evaluated based on Response Evaluation Criteria in Solid Tumours (RECIST) within a certain period of time. In a clinical trial, measuring the objective response rate is one way to evaluate the efficacy of a new treatment.

The term “Response Evaluation Criteria in Solid Tumours (RECIST)” refers to a set of guidelines used for assessing and evaluating the response of solid tumours to cancer therapeutics and treatment. Version 1.1 of the RECIST guidelines is referenced herein. The response to treatment is divided into four categories: complete response, partial response, stable disease and progressive disease based on the measurable parameters (tumor lesions and malignant lymph nodes) and non-measureable parameters such as ascites, pericardial effusion, abdominal organomegaly that are identified by physical exam.

As used herein, the term “complete response” refers to the disappearance of all tumors and/or sites of disease according to RECIST. A complete response can also be determined by the size of the lymphnodes, which should be less than 10 mm in the short axis. The evaluation of a complete response is detailed in RECIST version 1.1.

As used herein, the term “partial response” refers to a at least 30% decrease in the sum of diameters of tumours or target lesions, taking the baseline sum diameters as reference, the persistence of one of more tumours and/or sites of disease according to RECIST. Partial response can also be determined by the maintenance of higher-than-normal tumour marker levels.

As used herein, the term “stable disease” refers to cancer that is neither decreasing nor increasing in extent or severity. There is no sufficient shrinkage to qualify for partial response nor sufficient increase to qualify for progressive disease, taking as reference the smallest sum longest diameter (LD) since the treatment started according to RECIST.

As used herein, the term “progressive disease” refers to cancer that is growing, spreading, or getting worse. According to RECIST version 1.1, in progressive disease, at least a 20% increase in the sum of the longest diameter (LD) of target lesions, taking as reference the smallest sum longest diameter (LD) recorded since the treatment started or the appearance of one or more new lesions. In addition to the relative increase of 20%, the sum must also demonstrate an absolute increase of at least 5 mm. (

As used herein, the term “progression free survival (PFS)” refers to the length of time during and after the treatment of a disease, such as cancer, that a patient lives with the disease without deterioration. In a clinical trial, measuring the progression-free survival is one way to evaluate the efficacy of the new treatment.

As used herein, the term “Responders (Res)” refers to a stratified group of patients who showed a partial response or stable disease for 6 months or longer, according to guidelines established in the RECIST1.1.

As used herein, the term “Non-Responders (Non-res)” refers to a stratified group of patients who showed progressive disease within 6 months, according to guidelines established the RECIST1.1.

As used herein, the term “Peripheral blood mononuclear cells (PBMCs)” refer to cells isolated from peripheral blood and identified as blood cells with a round nucleus, which include, but not limited to, lymphocytes, monocytes, natural killer cells or dendritic cells.

As used herein, the term “cytometry by time-of-flight (CyTOF)” refers to a technology that measures the abundance of heavy metal isotope labels on antibodies and other tags (for example, but not limited to, peptide-MHC tetramers for labelling specific T cells) on single cells using mass spectrometry. CyTOF is applied to peripheral blood mononuclear cells (PBMC) for single-cell immunoprofiling.

As used herein, “single cell RNA sequencing (scRNA-seq)” or “single cell transcriptome sequencing” refers to a technique that examines the expression profiles of individual cells in a given population based on a next-generation sequencing platform. Single-cell RNA sequencing (scRNA-seq) is capable of revealing complex and rare cell populations, uncovering regulatory relationships between genes, and tracking the trajectories of distinct cell lineages in development.

As used herein, the terms “treatment-induced immune-related adverse event”, “treatment-induced irAE”, or “irAE” refer to the inflammatory side effects resulting from treatment with immune checkpoint inhibitors. Treatment-induced irAEs can be acute or chronic. Examples of treatment-induced irAEs include, but are not limited to, rash, inflammatory arthritis, myositis, vasculitis, colitis, hepatitis, psoriasis or a combination thereof. The severity of a treatment-induced immune-related adverse event can be evaluated based on National Cancer Institute Common Terminology Criteria for Adverse Events (NCI CTCAE) in to different gradings.

As used herein, the term “National Cancer Institute Common Terminology Criteria for Adverse Events (NCI CTCAE)” refers to a descriptive terminology utilized for adverse event reporting. As used herein, version 4.03 of the NCI CTCAE is referenced when categorizing the severity of treatment-induced immune related adverse event. Treatment-induced irAEs can be graded from Grade 1 (G1) to Grade 5 (G5) depending on the severity of the side effects based on criteria in the NCI CTCAE.

As used herein, the term “Tox” refers to a stratified group of patients who developed or experienced Grade 2 (G2) immune-related adverse event (irAE), or ≥G2 irAE, in response to anti-PD1 immune cell blockade therapy, based on classification in the NCI CTCAE.

As used herein, the term “Non-Tox” refers to a stratified group of hepatocellular carcinoma (HCC) patients who experienced Grade 1 or no immune-related adverse event (irAE) in response to anti-PD1 immune cell blockade therapy, based on classification in the NCI CTCAE.

The term “anti-cancer drugs” refers to drugs or therapeutic agents that promote cancer regression in a subject and prevent further tumour growth. Examples of anti-cancer drugs include, and are not limited to, TNFR2 inhibitor, Notch 1 inhibitor, anti-LTBR, anti-VEGFA, tyrosine kinase inhibitors (TKIs).

As used herein, the term “antigen presenting cells (APCs)” refers to a specialised group of immune cells that mediate the cellular immune response by processing and presenting antigens for recognition by certain lymphocytes such as T-cells. Classical APSCs include dendritic cells, macrophages, Langerhan cells and B cells.

As used herein, the term “type 1 conventional dendritic cells (cDC1)” refers to a subset of dendritic cells that are especially adept at presenting exogenous and endogenous antigen to T cells and regulating T cell proliferation survival and effector function.

The term “granzyme (GZM)” refers to a family of serine proteases traditionally known for their role in promoting cytotoxicity of foreign, infected or neoplastic cells. GZM induces cell death mediated by a collective of cytotoxic lymphocytes, for example, cytotoxic T cells and natural killer (NK) cells.

As used herein, the term “human leucocyte antigens (HLA)” refers to a type of molecule found on the surface of most cells in the body. Human leucocyte antigens (HLA) play an important part in the body's immune response to foreign substances. They make up a person's tissue type, which varies from person to person. Human leukocyte antigen (HLA) tests are done before a donor stem cell or organ transplant, to find out if tissues match between the donor and the person receiving the transplant. It is also known as human lymphocyte antigen.

As used herein, the term “lymphotoxin alpha (LTα)” refers to a cytokine produced by lymphocyte. LTα is a member of the tumor necrosis factor (TNF) superfamily of cytokine.

As used herein, the term “lymphotoxin beta receptor” refers to a receptor for the cytokine lymphotoxin alpha (LTα).

As used herein, the term “Mucosal-associated invariant T (MAIT) cells” refers to a population of unique innate-like T cells that bridge innate and adaptive immunity. They are activated by conserved bacterial ligands derived from vitamin B biosynthesis and have important roles in defence against bacterial and viral infections.

As used herein, the term “myeloid-derived suppressor cells (MDSC)” refers to pathologically activated neutrophils and monocytes with potent immunosuppressive activity that expand during cancer, inflammation and infection, and that have the ability to suppress T-cell responses.

As used herein, the term “effector memory T-cells (T_{EM})” refers to a subset of CXCR3⁺CD45RO⁺CD8⁺ memory T cells. Effector memory T-cells (T_{EM}) are long-lived and can quickly expand to large numbers of effector T cells upon re-exposure to their cognate antigen. By this mechanism they provide the immune system with “memory” against previously encountered pathogens. Effector memory T cells (T_{EM} cells) lack expression of CCR7 and L-selectin. They also have intermediate to high expression of CD44. Because these memory T cells lack the CCR7 lymph node-homing receptors they are usually found in the peripheral circulation and tissues.

As used herein, the term “T-helper cell (Tₕ)” refers to a specialized population of T-cells that express CD4 on their cell surface. They aid in the activity of other immune cells by releasing cytokines.

As used herein, the term “tumour mutational burden (TMB)” refers to the total number of mutations (changes) found in the DNA of cancer cells. Knowing the tumour mutational burden may help plan the best treatment. For example, tumours that have a high number of mutations appear to be more likely to respond to certain types of immunotherapies.

As used herein, the term “tumour microenvironment (TME)” refers to the normal cells, molecules, and blood vessels that surround and feed a tumour cell. A tumour can manipulate its microenvironment, and the microenvironment can affect how a tumour grows and spreads.

As used herein, the term “tumour necrosis factor receptor superfamily (TNFRSF)” refers a protein superfamily of cytokine receptors characterized by the ability to bind tumor necrosis factors.

As used herein, the term “Regulatory T cells (Treg)” refers to a specialized population of T cells that act to suppress an immune response, thereby contributing to immune homeostasis by maintaining unresponsiveness to self-antigen. It has been shown that Tregs are able to inhibit T cell proliferation and cytokine production and play a critical role in preventing autoimmunity.

The term “vascular endothelial growth factor-A (VEGF-A)” refers to a potent angiogenic factor that is upregulated in many tumors and contributes to tumor angiogenesis. As used herein, “anti-VEGFA” refers to a monoclonal VEGA antibody, which can be used as an anti-cancer drug.

The term “biomarker” refers to a biological molecule found in blood, other body fluids, or tissues that is a sign of a normal or abnormal process, or of a condition or disease. A biomarker may be used to see how well the body responds to a treatment for a disease or condition. As used herein, biomarkers can refer to biomarkers of response to immune checkpoint blockade treatment to predict outcome of immune checkpoint blockade treatment as well as biomarkers of immune checkpoint blockade-induced immune-related adverse events.

As used herein, the term “sample” refers to biological material obtained from a subject for analysis or testing purposes, for example, including, but not being limited to, a tissue sample or a bodily fluid sample. For example, the sample can be, but is not limited to cellular components of a liquid biopsy, amniotic fluid, bronchial lavage, cerebrospinal fluid, interstitial fluid, peritoneal fluids, pleural fluid, saliva, seminal fluid, urine, tears, peripheral blood, whole blood, plasma, and serum. In one example referred to herein the sample is obtained from peripheral blood mononuclear cells (PBMCs).

As used herein, the term “therapeutically effective amount” refers to an amount effective, at dosages and for periods of time necessary, to achieve a desired therapeutic result. A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects. A “therapeutically effective amount” for cancer therapy may also be measured by its ability to stabilize the progression of disease. The ability of a compound to inhibit cancer may be evaluated in an animal model system predictive of efficacy in treating human cancers.

### DETAILED DESCRIPTION OF THE PRESENT INVENTION

Immune checkpoint blockade (ICB) has achieved improving outcomes in treating cancer such as hepatocellular carcinoma (HCC). However, as the response in subject improves, the treatment induced immune-related adverse events (irAEs) also increase in tandem, resulting in fatality of the subjects or disruption of the treatment progress. Thus, what is needed is a method for predicting the response and treatment induced immune-related adverse events (irAEs) in a subject to receive, or is receiving an immune checkpoint blockade treatment, and a method of treating the subject with reduced treatment induced immune-related adverse events (irAEs).

The present disclosure investigates the coupling mechanism of the response and immune-related adverse events (irAEs) in liver cancer patients subjected to immunotherapy to identify biomarkers for predicting response and/or adverse events to an ICB treatment.

The following detailed description is merely exemplary in nature and is not intended to limit the invention or the application and uses of the invention. Furthermore, there is no intention to be bound by any theory presented in the preceding background of the invention or the following detailed description. It is also the intent of this invention to present a method for treating liver cancer, a method for predicting the response and/or treatment-induced immune-related adverse events (irAEs) in a liver cancer patient to receive, or is receiving an immune checkpoint blockade treatment.

In one aspect, the present disclosure provides a method of predicting occurrence of a response of a subject to a treatment by using a specific group of biomarkers which allow such a prediction. In other words, the method of the present disclosure allows by screening for specific biomarkers in a subject who were to receive, or is receiving an immune checkpoint blockade treatment for cancer to select those subjects who are more likely to show a positive response to the treatment. In one example, the screening of the subject is conducted early in the treatment. In another example, the screening of the subject is conducted before the treatment. The method as described herein also allows to determine those subjects who are less likely to positively respond to an immune checkpoint blockade treatment.

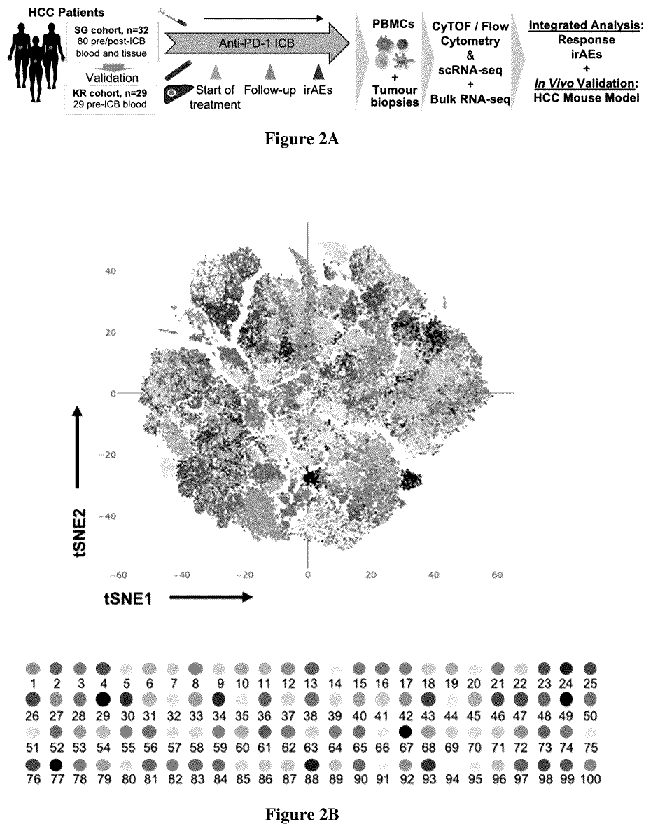

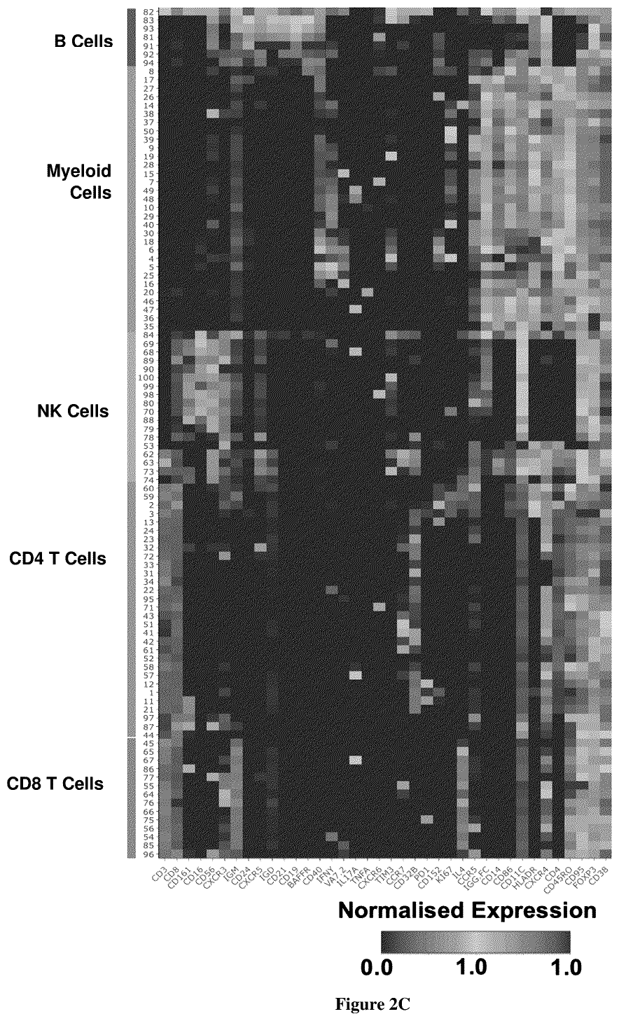

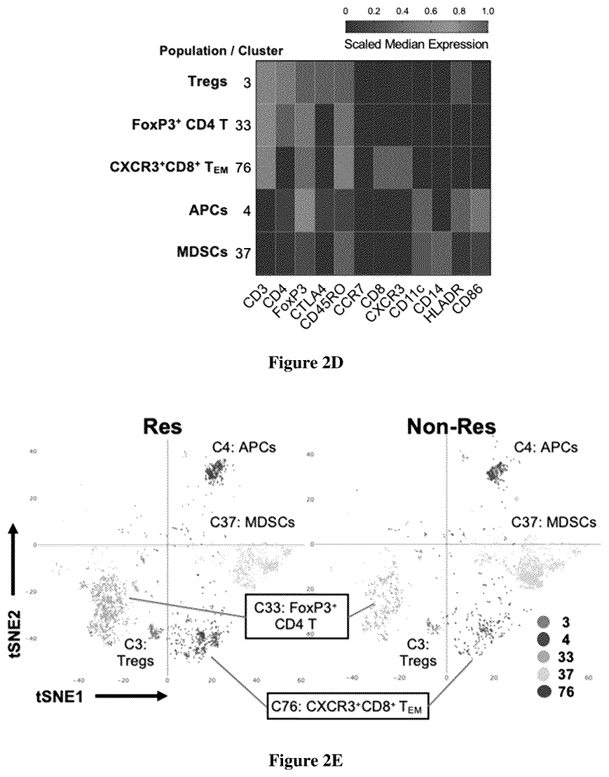

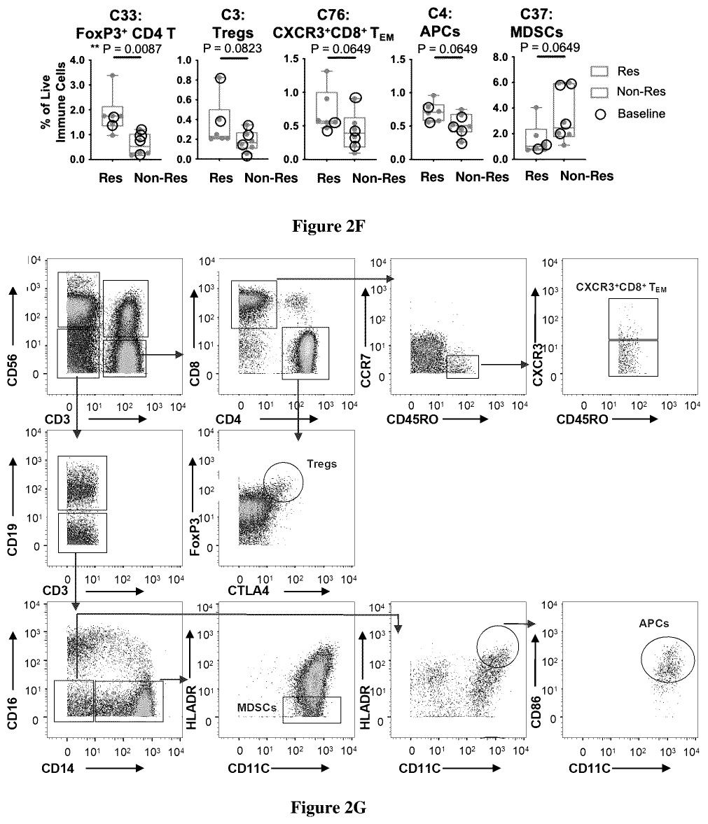

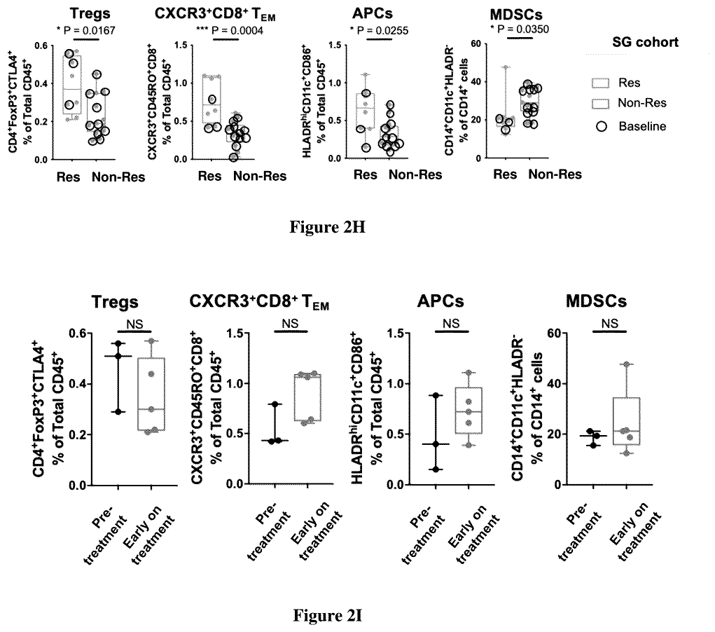

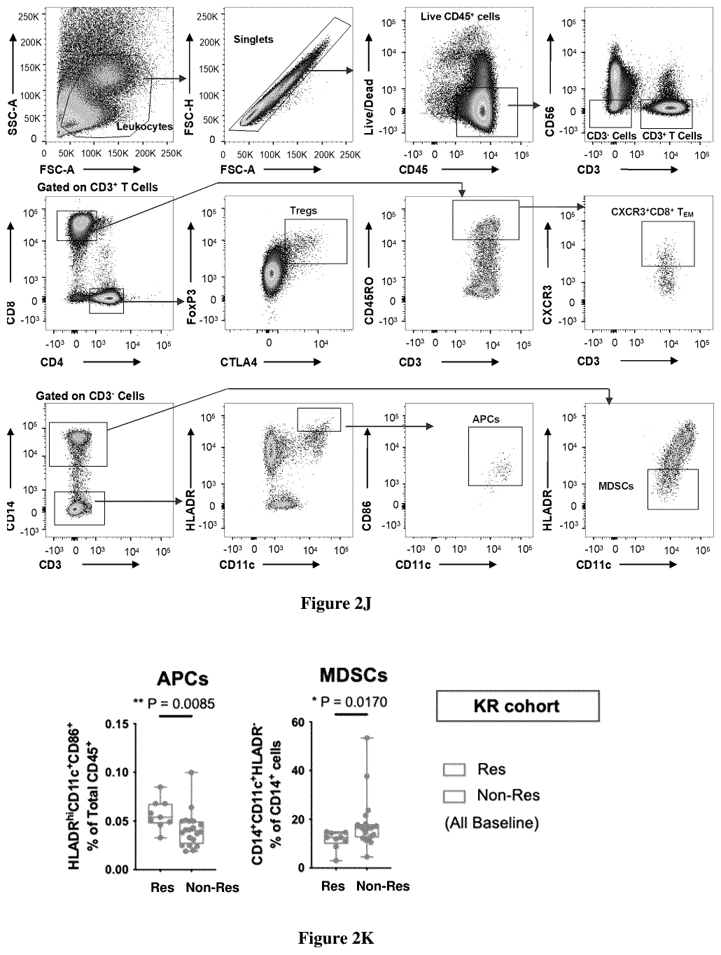

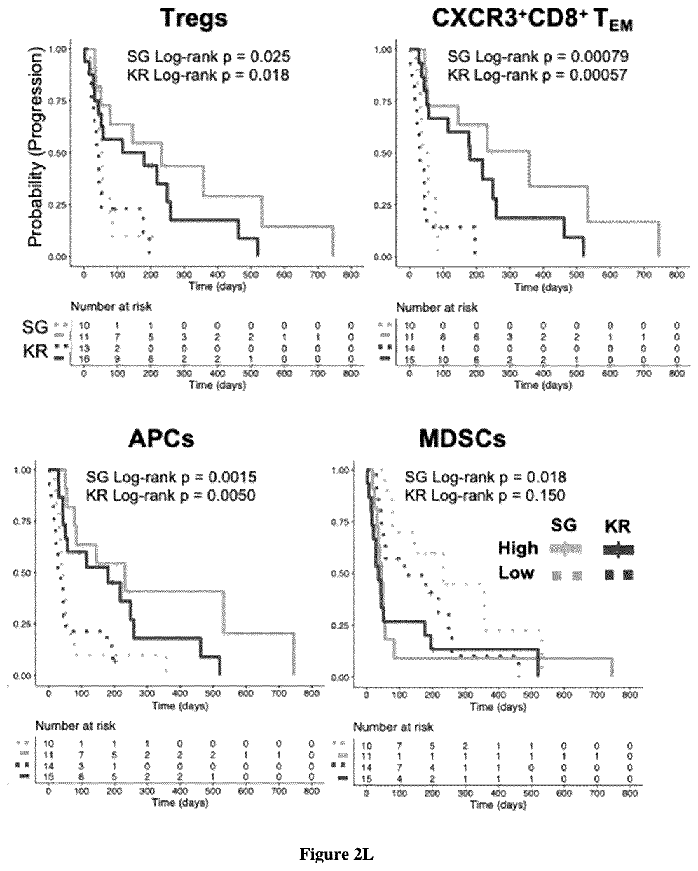

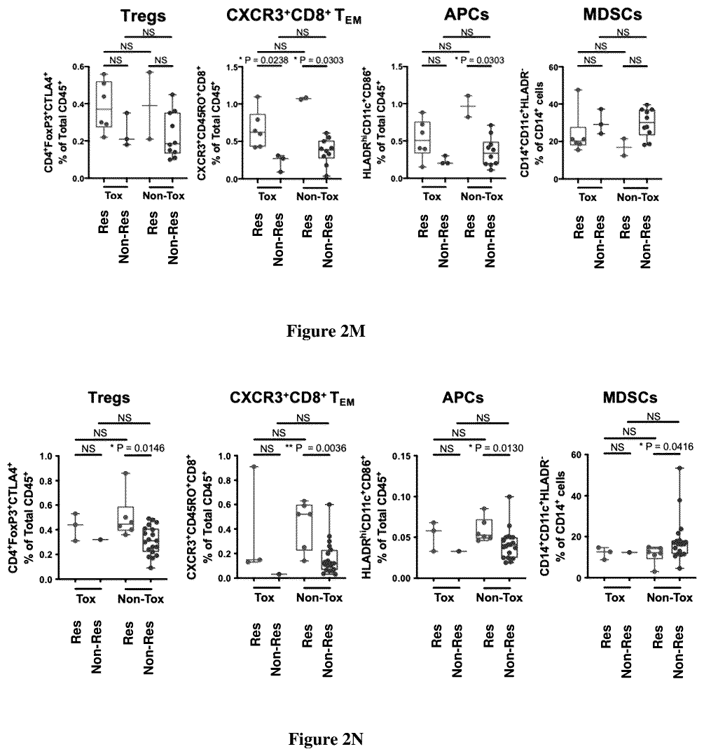

The response can be assessed based on the definitions according to Response Evaluation Criteria In Solid Tumours (RECIST) revised version 1.1. A person skilled in the art would be able to understand that the Response Evaluation Criteria In Solid Tumours (RECIST) may be revised over time and is able to extrapolate suitable adjustments in the criteria based on the revisions made in different versions. In one example, the response is a complete response. For example, disappearance of all tumors and/or sites of disease is observed in a complete response. A complete response can also be determined by the size of the lymph-nodes, which is <10 mm in the short axis. In another example, the response is a partial response. In a partial response, for example, at least a 30% decrease in the sum of diameters of tumours or target lesions is observed, taking as reference the baseline sum diameters. Based on the guidance of RECIST 1.1, a person skilled in the art is able to determine whether an observed response in a subject is a complete response or a partial response. As demonstrated in FIG. 2A, FIG. 2E and FIG. 2F, for example, the patients are stratified as Responders (Res), and Non-responders (Non-Res) accordingly.

In another aspect, the present disclosure provides a method of predicting occurrence of one or more treatment-induced immune-related adverse events (irAEs) in a subject, if the subject were to receive an immune checkpoint inhibitor treatment. In another example, the present disclosure provides a method of predicting occurrence of one or more treatment-induced immune-related adverse events (irAEs) in a subject, if the subject is receiving, or just started receiving an immune checkpoint inhibitor treatment. The method of the present disclosure allows screening of subjects who are less likely to show treatment-induced immune-related adverse events (irAEs) after receiving an immune checkpoint blockade treatment for cancer by detecting the presence or absence of specific sets of biomarkers before or during the immune checkpoint blockade treatment. In one example, the screening of the subject is conducted early in the treatment. In another example, the screening of the subject is conducted before the treatment. The method as described herein also allows to determine those subjects who are likely to show treatment-induced immune-related adverse events (irAEs) when receiving an immune checkpoint blockade treatment.

In one example, the treatment-induced immune-related adverse event (irAE) is an adverse effect induced by the treatment with one or more immune checkpoint inhibitors. In another example, the treatment-induced immune-related adverse event (irAE) is an inflammatory side effect. In one example, the treatment-induced irAE can be acute or chronic. In another example, the treatment-induced irAE may comprise symptoms that include, but are not limited to, rash, inflammatory arthritis, myositis, vasculitis, colitis, hepatitis, psoriasis or a combination thereof. It is known that treatment-induced irAEs can be graded depending on the severity of the side effects. For example, a person skilled in the art can determine the severity of the side effects (“grade”) according to the Common Terminology Criteria for Adverse Events (CTCAE). In one example, the grading is based on Common Terminology Criteria for Adverse Events (CTCAE) version 4.03. A person skilled in the art would be able to understand that the Common Terminology Criteria for Adverse Events (CTCAE) may be revised over time and is able to extrapolate suitable adjustments in the criteria based on the revisions made in different versions. In one example, the subject is suffering from Grade 2 irAEs. In another example, the subject is suffering from irAEs of Grade 2 and above. In another example, the subject is suffering from irAEs of above Grade 1. In another example, the subject is suffering from irAEs of Grade 1 or below. In a further example, the subject is not suffering from any irAEs. According to the Criteria for Adverse Events (CTCAE), in the case of irAEs of Grade 2, therapeutic interventions are considered. In FIG. 2M, for example, the patients are grouped into Tox and Non-Tox groups based on their immune-related adverse events (irAEs) status. In one example, patients showing ≥Grade 2 irAEs are included in the Tox group while patients of Grade 1 or no irAEs are in Non-Tox group.

In one example, the subject is a mammal. In another example, the subject is a human. In another example, the subject is a cancer patient. In some other examples, the subject is suspected to suffer from cancer.

In another example, the cancer is a liver cancer. In further examples, the cancer can be, but is not limited to hepatocellular carcinoma, cholangiocarcinoma, and hepatoblastoma.

In one example, the treatment is an immunotherapy. In another example, the immunotherapy is a combination therapy. In another example, the treatment is an immune checkpoint blockade treatment. In yet another example, the treatment comprises administration of one or more immune checkpoint inhibitors to the subject. Immune checkpoint inhibitors targeting various checkpoint proteins such as CTLA-4 (cytotoxic T lymphocyte associated protein 4), PD-1 (programmed cell death protein 1) and PD-L1 (programmed cell death ligand 1) for treatment of cancer are known in the art. In some cases, the immune checkpoint inhibitors are antibodies that specifically interact and inhibit the immune checkpoint proteins. One example for immune checkpoint inhibitors commonly used in therapy is monoclonal antibody. For example, checkpoint inhibitors that block PD-1 include, but are not limited to nivolumab (ATC code: L01FF01) and pembrolizumab (ATC code: L01FF02). In another example, ipilimumab (ATC code: L01FX04) is a checkpoint inhibitor drug that blocks CTLA-4. In a further example, checkpoint inhibitors that block PD-L1 include, but are not limited to: atezolizumab, avelumab, durvalumab. As demonstrated in FIG. 2A, the analysis presented herein is based on the clinical data in two independent patient cohorts, i.e. a Singaporean cohort and a Korean cohort. Both of the patient cohorts receive checkpoint inhibitor drug treatment. For example, both cohorts receive an anti-PD-1 treatment. In some examples, the patients receive nivolumab treatment. In some further examples, the patients receive pembrolizumab treatment. Other antibodies specifically targeting and inhibiting one or more immune checkpoint proteins can be used as immune checkpoint blockades.

In one example, the present disclosure provides a method of predicting the occurrence of a complete or partial response in a subject suffering from liver cancer if the subject were to receive an immune checkpoint inhibitor treatment. In one example, the present disclosure provides a method of predicting the occurrence of a complete or partial response in a subject suffering from liver cancer if the subject is receiving an immune checkpoint inhibitor treatment. In another example, the present disclosure provides a method of predicting occurrence of one or more treatment-induced immune-related adverse events (irAEs) in a subject suffering from liver cancer if the subject were to receive an immune checkpoint inhibitor treatment. In another example, the present disclosure provides a method of predicting occurrence of one or more treatment-induced immune-related adverse events (irAEs) in a subject suffering from liver cancer if the subject is receiving an immune checkpoint inhibitor treatment. In some examples, the method comprising detecting the presence of an immune cell population. An immune cell is a cell that is part of the immune system and helps the body against infections and other diseases. Immune cells are developed from stem cells in the bone marrow and become different types of white blood cells including neutrophils, eosinophils, basophils, mast cells, monocytes, macrophages, dendritic cells, natural killer cells, and lymphocytes (B cells and T cells). Immune cells can be further classified based on the surface biomarkers, indicating the class, cell type, and subtypes of the cell. For example, leukocytes in general comprise positive CD45 surface biomarker while monocytes cell type has a biomarker signature of CD14⁺HLA-DR⁺CD206⁻CD86⁻.

Methods of detecting the cell surface biomarkers are well known in the art. For example, flow cytometry, immunohistochemistry, proteomic profiling, genetic profiling, and next generation sequencing (NGS). Exemplary protocols for carrying out these experiments are provided in the Experiment Section. A person skilled in the art is capable of utilising the available protocols with minimal modifications to conduct these known methods with reasonable expectation of success.

In some examples, the immune cell population comprises one or more biomarkers for predicting occurrence of a response of a subject before or during an immune checkpoint inhibitor treatment. In one example, the biomarker is CXCR3. In another example, the biomarker is CD45RO. In another example, the biomarker is CCR7. In another example, the biomarker is CD8. In another example, the biomarker is HLADR. In another example, the biomarker is ITGAX (CD11c). In another example, the biomarker is CD86. In one example, the one or more biomarkers can be, but are not limited to: CXCR3, CD45RO; CXCR3, CCR7; CXCR3, CD8; CXCR3, HLADR; CXCR3, ITGAX; CXCR3, CD86; CD45RO, CCR7; CD45RO, CD8; CD45RO, HLADR; CD45RO, ITGAX; CD45RO, CD86; CCR7, CD8; CCR7, HLADR; CCR7, ITGAX; CCR7, CD86; CD8, HLADR; CD8, ITGAX; CD8, CD86; HLADR, ITGAX; HLADR, CD86; and ITGAX, CD86. In one example, the one or more biomarkers can be, but are not limited to: CXCR3, CD45RO, CCR7; CXCR3, CD45RO, CD8; CXCR3, CD45RO, HLADR; CXCR3, CD45RO, ITGAX; CXCR3, CD45RO, CD86; CXCR3, CCR7, CD8; CXCR3, CCR7, HLADR; CXCR3, CCR7, ITGAX; CXCR3, CCR7, CD86; CXCR3, CD8, HLADR; CXCR3, CD8, ITGAX; CXCR3, CD8, CD86; CXCR3, HLADR, ITGAX; CXCR3, HLADR, CD86; CXCR3, ITGAX, CD86; CD45RO, CCR7, CD8; CD45RO, CCR7, HLADR; CD45RO, CCR7, ITGAX; CD45RO, CCR7, CD86; CD45RO, CD8, HLADR; CD45RO, CD8, ITGAX; CD45RO, CD8, CD86; CD45RO, HLADR, ITGAX; CD45RO, HLADR, CD86; CD45RO, ITGAX, CD86; CCR7, CD8, HLADR; CCR7, CD8, ITGAX; CCR7, CD8, CD86; CCR7, HLADR, ITGAX; CCR7, HLADR, CD86; CCR7, ITGAX, CD86; CD8, HLADR, ITGAX; CD8, HLADR, CD86; CD8, ITGAX, CD86; and HLADR, ITGAX, CD86. In one example, the one or more biomarkers can be, but are not limited to: CXCR3, CD45RO, CCR7, CD8; CXCR3, CD45RO, CCR7, HLADR; CXCR3, CD45RO, CCR7, ITGAX; CXCR3, CD45RO, CCR7, CD86; CXCR3, CD45RO, CD8, HLADR; CXCR3, CD45RO, CD8, ITGAX; CXCR3, CD45RO, CD8, CD86; CXCR3, CD45RO, HLADR, ITGAX; CXCR3, CD45RO, HLADR, CD86; CXCR3, CD45RO, ITGAX, CD86; CXCR3, CCR7, CD8, HLADR; CXCR3, CCR7, CD8, ITGAX; CXCR3, CCR7, CD8, CD86; CXCR3, CCR7, HLADR, ITGAX; CXCR3, CCR7, HLADR, CD86; CXCR3, CCR7, ITGAX, CD86; CXCR3, CD8, HLADR, ITGAX; CXCR3, CD8, HLADR, CD86; CXCR3, CD8, ITGAX, CD86; CXCR3, HLADR, ITGAX, CD86; CD45RO, CCR7, CD8, HLADR; CD45RO, CCR7, CD8, ITGAX; CD45RO, CCR7, CD8, CD86; CD45RO, CCR7, HLADR, ITGAX; CD45RO, CCR7, HLADR, CD86; CD45RO, CCR7, ITGAX, CD86; CD45RO, CD8, HLADR, ITGAX; CD45RO, CD8, HLADR, CD86; CD45RO, CD8, ITGAX, CD86; CD45RO, HLADR, ITGAX, CD86; CCR7, CD8, HLADR, ITGAX; CCR7, CD8, HLADR, CD86; CCR7, CD8, ITGAX, CD86; CCR7, HLADR, ITGAX, CD86; and CD8, HLADR, ITGAX, CD86. In one example, the one or more biomarkers can be, but are not limited to: CXCR3, CD45RO, CCR7, CD8, HLADR; CXCR3, CD45RO, CCR7, CD8, ITGAX; CXCR3, CD45RO, CCR7, CD8, CD86; CXCR3, CD45RO, CCR7, HLADR, ITGAX; CXCR3, CD45RO, CCR7, HLADR, CD86; CXCR3, CD45RO, CCR7, ITGAX, CD86; CXCR3, CD45RO, CD8, HLADR, ITGAX; CXCR3, CD45RO, CD8, HLADR, CD86; CXCR3, CD45RO, CD8, ITGAX, CD86; CXCR3, CD45RO, HLADR, ITGAX, CD86; CXCR3, CCR7, CD8, HLADR, ITGAX; CXCR3, CCR7, CD8, HLADR, CD86; CXCR3, CCR7, CD8, ITGAX, CD86; CXCR3, CCR7, HLADR, ITGAX, CD86; CXCR3, CD8, HLADR, ITGAX, CD86; CD45RO, CCR7, CD8, HLADR, ITGAX; CD45RO, CCR7, CD8, HLADR, CD86; CD45RO, CCR7, CD8, ITGAX, CD86; CD45RO, CCR7, HLADR, ITGAX, CD86; CD45RO, CD8, HLADR, ITGAX, CD86; and CCR7, CD8, HLADR, ITGAX, CD86. In one example, the one or more biomarkers can be, but are not limited to: CXCR3, CD45RO, CCR7, CD8, HLADR, ITGAX; CXCR3, CD45RO, CCR7, CD8, HLADR, CD86; CXCR3, CD45RO, CCR7, CD8, ITGAX, CD86; CXCR3, CD45RO, CCR7, HLADR, ITGAX, CD86; CXCR3, CD45RO, CD8, HLADR, ITGAX, CD86; CXCR3, CCR7, CD8, HLADR, ITGAX, CD86; and CD45RO, CCR7, CD8, HLADR, ITGAX, CD86. In another example, the one or more biomarkers are CXCR3, CD45RO, CCR7, CD8, HLADR, ITGAX (CD11c), and CD86. In another example, the one or more biomarkers can be, but are not limited to CXCR3, CD45RO, CCR7, CD8, HLADR, ITGAX (CD11c), and CD86.

In one example, the immune cell population comprises a CXCR3⁺CD45RO⁺CD8⁺ effector memory T (T_{EM}) cell population. In another example, the immune cell population comprises a ITGAX(CD11c)⁺HLADR⁺CD86⁺ antigen presenting cell (APC) population. As demonstrated in FIG. 2H, significant enrichment of the immune subsets of Tregs, CXCR3+CD8+ T_{EM} cells and APCs are found in responders (Res). In another example, wherein the CXCR3⁺CD45RO⁺CD8⁺ effector memory T (T_{EM}) cell population is characterized by an absence of CCR7. In another example, wherein the CXCR3⁺CD45RO⁺CD8⁺ effector memory T (T_{EM}) cell population does not express CCR7 marker.

In another example, the present disclosure provides a method of predicting occurrence of a complete or partial response before or during an immune checkpoint inhibitor treatment in a subject suffering from liver cancer, wherein the method comprises detecting an immune cell population that comprises one or more biomarkers selected from the group consisting of CXCR3, CD45RO, CCR7, CD8, HLADR, ITGAX (CD11c), and CD86 in a sample obtained from the subject, wherein the detection of the one or more biomarkers indicates that the treatment of the subject with an immune checkpoint inhibitor results in a complete or partial response in the subject. In another example, the present disclosure provides a method of predicting occurrence of a complete or partial response before or during an immune checkpoint inhibitor treatment in a subject suffering from liver cancer, wherein the method comprises detecting an immune cell population that comprises one or more biomarkers selected from the group consisting of CXCR3, CD45RO, CCR7, CD8, HLADR, ITGAX (CD11c), and CD86 in a sample obtained from the subject, wherein the non-detection of the one or more biomarkers indicates that the treatment of the subject with an immune checkpoint inhibitor does not result in a complete or partial response in the subject. In some examples, the immune cell population comprises one or more biomarkers for predicting one or more treatment-induced immune-related adverse events (irAEs) in a subject. In one example, the biomarker is CXCR3. In another example, the biomarker is CD45RO. In another example, the biomarker is CCR7. In another example, the biomarker is CD8. In another example, the biomarker is HLADR. In another example, the biomarker is CD86. In another example, the biomarker is CD14. In one example, the one or more biomarkers can be, but are not limited to: CXCR3, CD45RO; CXCR3, CCR7; CXCR3, CD8; CXCR3, HLADR; CXCR3, CD14; CXCR3, CD86; CD45RO, CCR7; CD45RO, CD8; CD45RO, HLADR; CD45RO, CD14; CD45RO, CD86; CCR7, CD8; CCR7, HLADR; CCR7, CD14; CCR7, CD86; CD8, HLADR; CD8, CD14; CD8, CD86; HLADR, CD14; HLADR, CD86; and CD14, CD86. In one example, the one or more biomarkers can be, but are not limited to: CXCR3, CD45RO, CCR7; CXCR3, CD45RO, CD8; CXCR3, CD45RO, HLADR; CXCR3, CD45RO, CD14; CXCR3, CD45RO, CD86; CXCR3, CCR7, CD8; CXCR3, CCR7, HLADR; CXCR3, CCR7, CD14; CXCR3, CCR7, CD86; CXCR3, CD8, HLADR; CXCR3, CD8, CD14; CXCR3, CD8, CD86; CXCR3, HLADR, CD14; CXCR3, HLADR, CD86; CXCR3, CD14, CD86; CD45RO, CCR7, CD8; CD45RO, CCR7, HLADR; CD45RO, CCR7, CD14; CD45RO, CCR7, CD86; CD45RO, CD8, HLADR; CD45RO, CD8, CD14; CD45RO, CD8, CD86; CD45RO, HLADR, CD14; CD45RO, HLADR, CD86; CD45RO, CD14, CD86; CCR7, CD8, HLADR; CCR7, CD8, CD14; CCR7, CD8, CD86; CCR7, HLADR, CD14; CCR7, HLADR, CD86; CCR7, CD14, CD86; CD8, HLADR, CD14; CD8, HLADR, CD86; CD8, CD14, CD86; and HLADR, CD14, CD86. In one example, the one or more biomarkers can be, but are not limited to: CXCR3, CD45RO, CCR7, CD8; CXCR3, CD45RO, CCR7, HLADR; CXCR3, CD45RO, CCR7, CD14; CXCR3, CD45RO, CCR7, CD86; CXCR3, CD45RO, CD8, HLADR; CXCR3, CD45RO, CD8, CD14; CXCR3, CD45RO, CD8, CD86; CXCR3, CD45RO, HLADR, CD14; CXCR3, CD45RO, HLADR, CD86; CXCR3, CD45RO, CD14, CD86; CXCR3, CCR7, CD8, HLADR; CXCR3, CCR7, CD8, CD14; CXCR3, CCR7, CD8, CD86; CXCR3, CCR7, HLADR, CD14; CXCR3, CCR7, HLADR, CD86; CXCR3, CCR7, CD14, CD86; CXCR3, CD8, HLADR, CD14; CXCR3, CD8, HLADR, CD86; CXCR3, CD8, CD14, CD86; CXCR3, HLADR, CD14, CD86; CD45RO, CCR7, CD8, HLADR; CD45RO, CCR7, CD8, CD14; CD45RO, CCR7, CD8, CD86; CD45RO, CCR7, HLADR, CD14; CD45RO, CCR7, HLADR, CD86; CD45RO, CCR7, CD14, CD86; CD45RO, CD8, HLADR, CD14; CD45RO, CD8, HLADR, CD86; CD45RO, CD8, CD14, CD86; CD45RO, HLADR, CD14, CD86; CCR7, CD8, HLADR, CD14; CCR7, CD8, HLADR, CD86; CCR7, CD8, CD14, CD86; CCR7, HLADR, CD14, CD86; and CD8, HLADR, CD14, CD86. In one example, the one or more biomarkers can be, but are not limited to: CXCR3, CD45RO, CCR7, CD8, HLADR; CXCR3, CD45RO, CCR7, CD8, CD14; CXCR3, CD45RO, CCR7, CD8, CD86; CXCR3, CD45RO, CCR7, HLADR, CD14; CXCR3, CD45RO, CCR7, HLADR, CD86; CXCR3, CD45RO, CCR7, CD14, CD86; CXCR3, CD45RO, CD8, HLADR, CD14; CXCR3, CD45RO, CD8, HLADR, CD86; CXCR3, CD45RO, CD8, CD14, CD86; CXCR3, CD45RO, HLADR, CD14, CD86; CXCR3, CCR7, CD8, HLADR, CD14; CXCR3, CCR7, CD8, HLADR, CD86; CXCR3, CCR7, CD8, CD14, CD86; CXCR3, CCR7, HLADR, CD14, CD86; CXCR3, CD8, HLADR, CD14, CD86; CD45RO, CCR7, CD8, HLADR, CD14; CD45RO, CCR7, CD8, HLADR, CD86; CD45RO, CCR7, CD8, CD14, CD86; CD45RO, CCR7, HLADR, CD14, CD86; CD45RO, CD8, HLADR, CD14, CD86; and CCR7, CD8, HLADR, CD14, CD86. In one example, the one or more biomarkers can be, but are not limited to: CXCR3, CD45RO, CCR7, CD8, HLADR, CD14; CXCR3, CD45RO, CCR7, CD8, HLADR, CD86; CXCR3, CD45RO, CCR7, CD8, CD14, CD86; CXCR3, CD45RO, CCR7, HLADR, CD14, CD86; CXCR3, CD45RO, CD8, HLADR, CD14, CD86; CXCR3, CCR7, CD8, HLADR, CD14, CD86; and CD45RO, CCR7, CD8, HLADR, CD14, CD86. In another example, the one or more biomarkers are CXCR3, CD45RO, CCR7, CD8, HLADR, CD14, and CD86. In another example, the one or more biomarkers can be, but are not limited to CXCR3, CD45RO, CCR7, CD8, HLADR, CD14, and CD86.

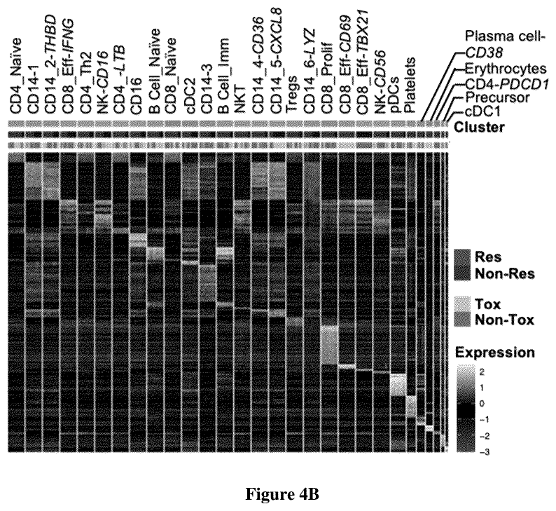

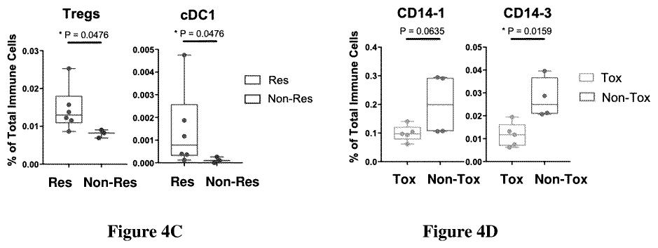

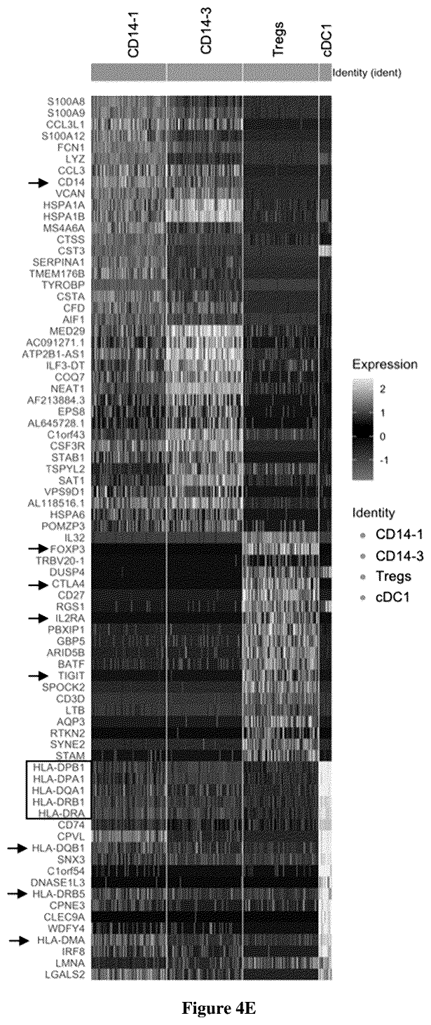

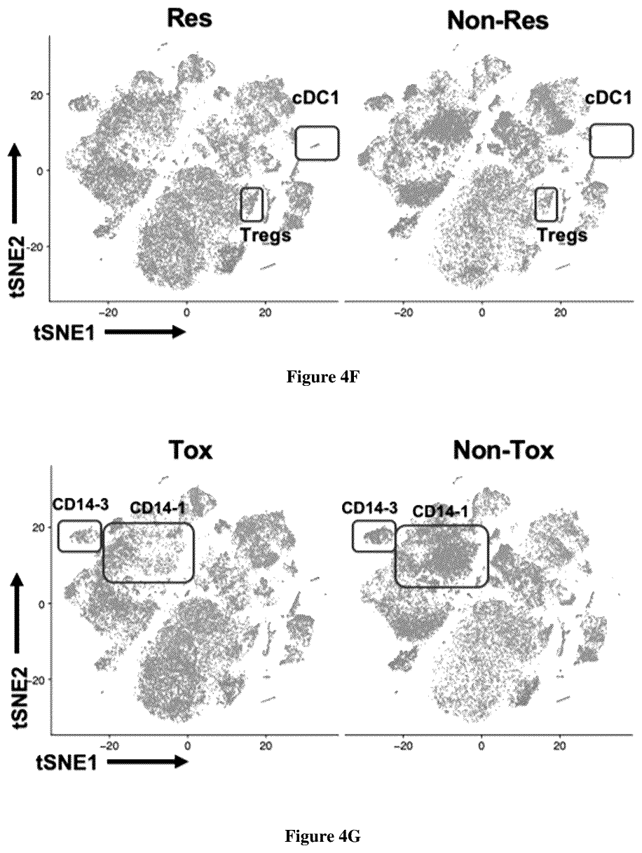

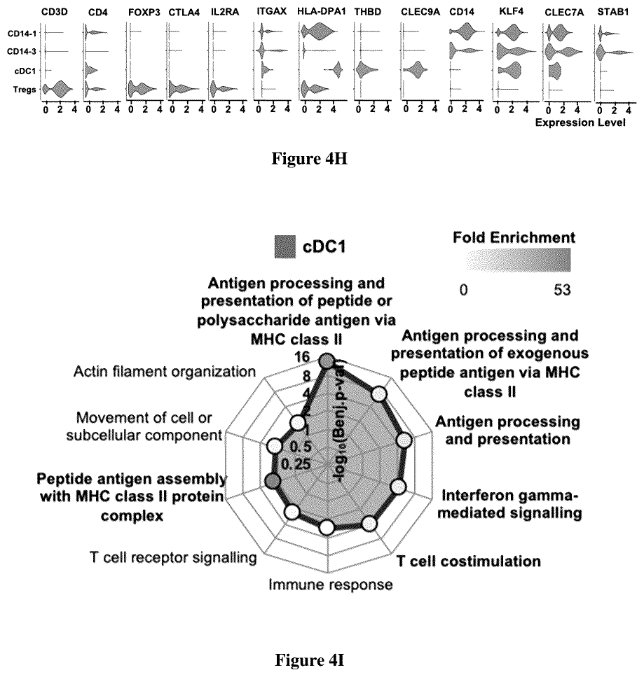

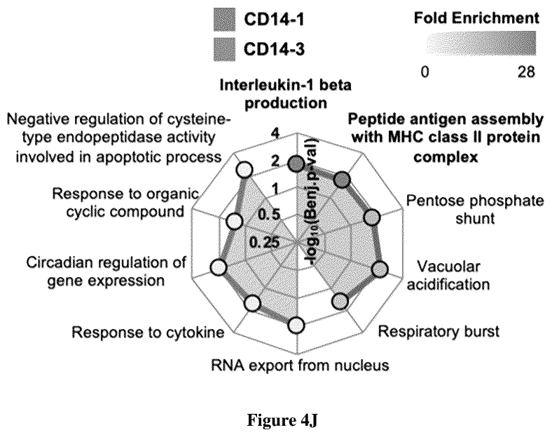

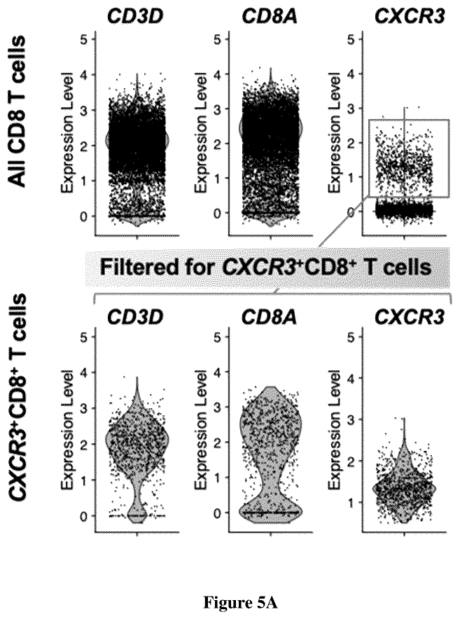

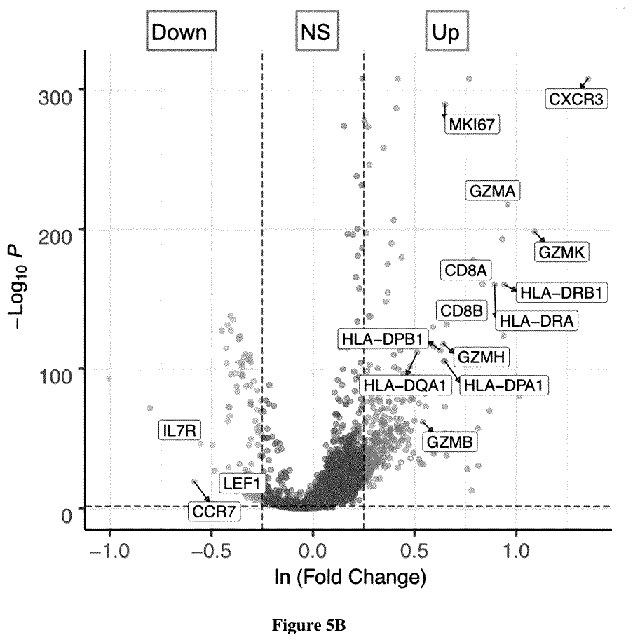

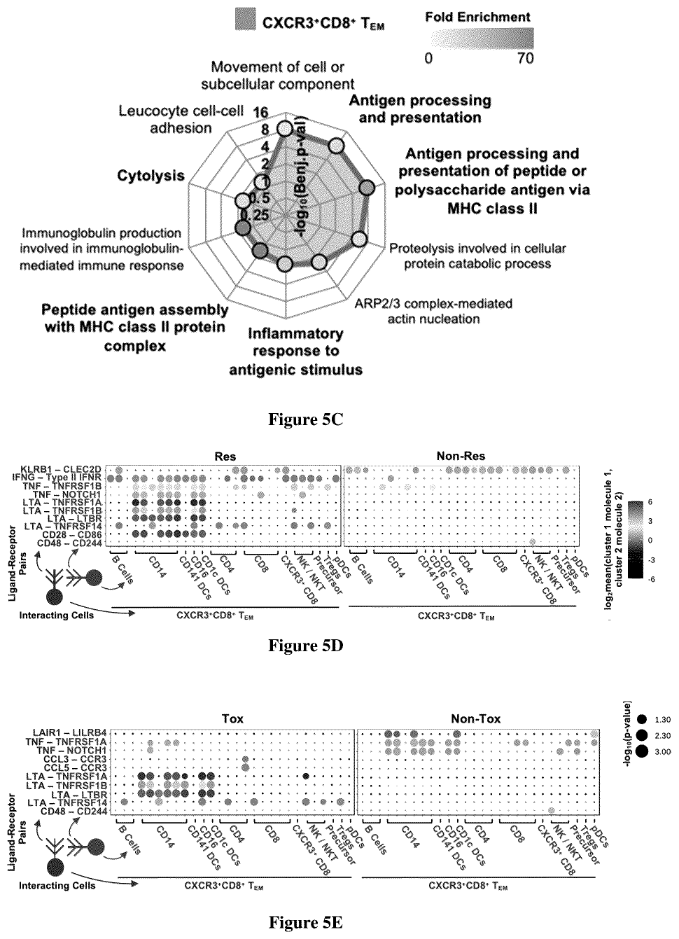

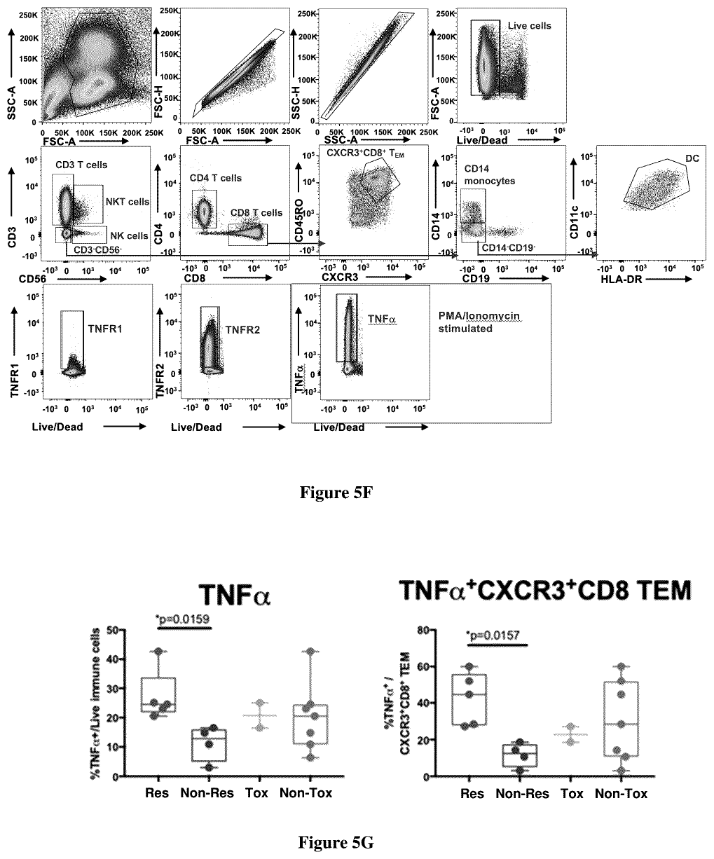

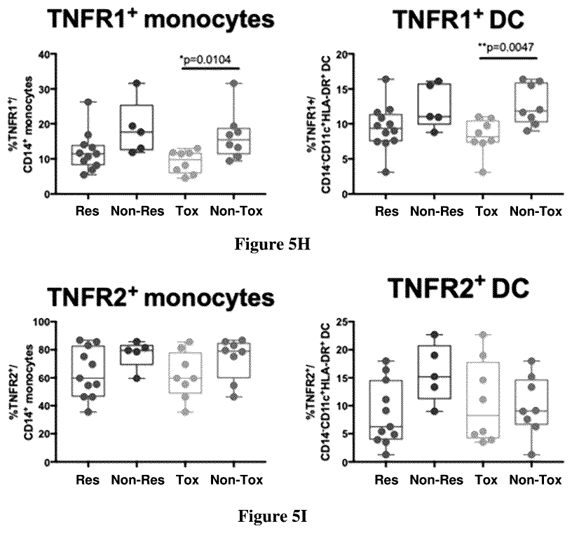

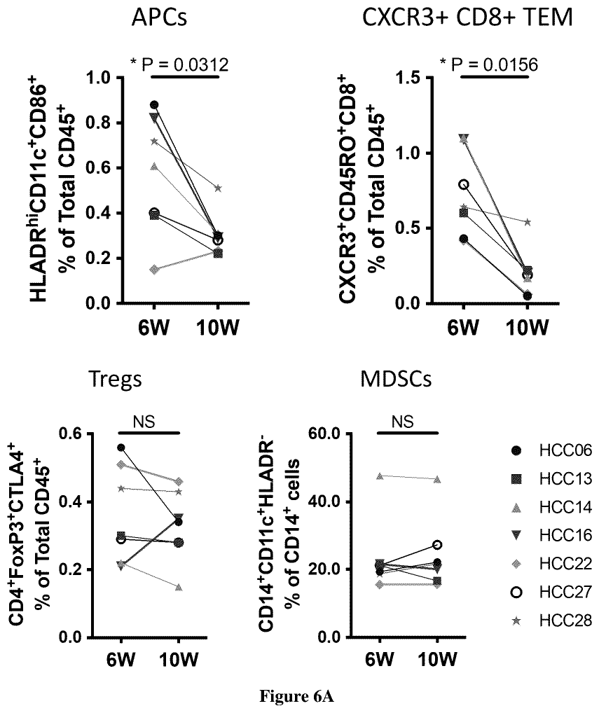

In another example, the present disclosure provides a method of predicting the occurrence of one or more treatment-induced immune-related adverse events (irAEs) in a subject suffering from liver cancer, if the subject were to receive, or is receiving an immune checkpoint inhibitor treatment, wherein the method comprises detecting an immune cell population that comprises one or more biomarkers selected from the group consisting of CXCR3, CD45RO, CCR7, CD8, HLADR, CD86, and CD14 in a sample obtained from the subject, wherein the detection of the one or more biomarkers indicates that the treatment of the subject with an immune checkpoint inhibitor does not result in one or more treatment-induced immune-related adverse events (irAEs) in the subject. As exemplarily supported by FIG. 4H and FIG. 5C, CXCR3⁺CD45RO⁺CD8⁺ effector memory T (T_{EM}) cell and CD14⁺HLADR⁺CD86⁺ antigen presenting cell (APC) are representative for prediction of treatment-induced immune-related adverse events (irAEs). In another example, the CXCR3⁺CD45RO⁺CD8⁺ effector memory T (T_{EM}) cell population is characterized by an absence of CCR7. In another example, the CXCR3⁺CD45RO⁺CD8⁺ effector memory T (T_{EM}) cell population does not express the CCR7 marker.

In another aspect, the present disclosure provides a method of treating liver cancer in a subject. In one example, the present disclosure provides a method of treating liver cancer in a subject comprising detecting the immune cell population as disclosed herein before treatment of the subject. In another example, the present disclosure provides a method of treating liver cancer in a subject, comprising detecting the immune cell population as disclosed herein before or during the treatment of the subject and administering a therapeutically effective amount of an immune checkpoint inhibitor to the subject if the immune cell population is detected. In another example, the present disclosure provides a method of treating liver cancer in a subject, comprising detecting an immune cell population that comprises one or more biomarkers selected from the group consisting of CXCR3, CD45RO, CCR7, CD8, HLADR, ITGAX (CD11c), CD14, and CD86 in a sample obtained from the subject before or during the treatment of the subject. In another example, the present disclosure provides a method of treating liver cancer in a subject, comprising detecting an immune cell population that comprises one or more biomarkers selected from the group consisting of CXCR3, CD45RO, CCR7, CD8, HLADR, ITGAX (CD11c), and CD86 in a sample obtained from the subject. In one example of the method as disclosed herein, in case of the detection of the immune cell population comprises one or more biomarkers which can be, but are not limited to CXCR3, CD45RO, CCR7, CD8, HLADR, ITGAX (CD11c), and CD86 in the sample obtained from the subject, the detection of the one or more or all biomarkers indicates that the administration of the therapeutically effective amount of an immune checkpoint inhibitor results in a complete or partial response in the subject, and the therapeutically effective amount of an immune checkpoint inhibitor is administered to the subject, or is continued for administration to the subject. In another example of the method as disclosed herein, in case the immune cell population does not comprises one or more biomarkers which can be, but are not limited to CXCR3, CD45RO, CCR7, CD8, HLADR, ITGAX (CD11c), and CD86 in the sample obtained from the subject, the non-detection of the one or more or all biomarkers indicates that the administration of the therapeutically effective amount of an immune checkpoint inhibitor will not result in a complete or partial response in the subject, and the therapeutically effective amount of an immune checkpoint inhibitor is not administered to the subject, or is discontinued from administration to the subject.

In one example of the method as disclosed herein, the detection of the immune cell population comprises one or more biomarkers selected from the group consisting of CXCR3, CD45RO, CCR7, CD8, HLADR, CD14, and CD86 in the sample obtained from the subject indicates that the administration of the therapeutically effective amount of an immune checkpoint inhibitor will not result in one or more treatment-induced immune-related adverse events (irAEs) in the subject, and the therapeutically effective amount of an immune checkpoint inhibitor is administered to the subject, or is continued for administration to the subject. In another example of the method as disclosed herein, wherein the immune cell population comprises one or more biomarkers selected from the group consisting of CXCR3, CD45RO, CCR7, CD8, HLADR, CD14, and CD86 in the sample obtained from the subject is not detected, indicating that the administration of the therapeutically effective amount of an immune checkpoint inhibitor results in one or more treatment-induced immune-related adverse events (irAEs) in the subject, and the therapeutically effective amount of an immune checkpoint inhibitor is not administered to the subject, or is discontinued from administration to the subject.

In another example, the present disclosure provides a method of treating liver cancer in a subject, comprising detecting an immune cell population that comprises one or more biomarkers selected from the group consisting of CXCR3, CD45RO, CCR7, CD8, HLADR, ITGAX (CD11c), and CD86 in a sample obtained from the subject; and administering a therapeutically effective amount of an immune checkpoint inhibitor to the subject if the immune cell population is detected in the sample. In one example, method of treating liver cancer in a subject further administering one or more anti-cancer drugs to the subject. In another example, the therapeutically effective amount is an amount effective, at dosages and for periods of time necessary, to achieve a desired therapeutic result, such as treating liver cancer. A person skilled in the art would be able to routinely adjust the amount of the immune checkpoint inhibitors based on factors such as the route of administration, subject's body size, and severity of the subject's symptoms.

In another aspect, the present disclosure provides the use of an immune checkpoint inhibitor in the manufacture of a medicament for treating liver cancer in a subject. In one example, the use comprises detecting the immune cell population as defined herein. In another example, the use further comprises that one or more anti-cancer drugs are to be administered to the subject.

In another aspect, the present disclosure provides an immune checkpoint inhibitor for treating liver cancer in a subject. In one example, the immune checkpoint inhibitor for treating liver cancer comprises detecting the immune cell population as defined herein. In another example, the present disclosure provides an immune checkpoint inhibitor for treating liver cancer in a subject, comprising detecting an immune cell population, wherein the immune cell immune population comprises one or more biomarkers selected from the group consisting of CXCR3, CD45RO, CCR7, CD8, HLADR, CD86, and CD14 in a sample obtained from the subject. In another example, the present disclosure provides an immune checkpoint inhibitor for treating liver cancer in a subject, comprising detecting an immune cell population, wherein the immune cell immune population comprises one or more biomarkers selected from the group consisting of CXCR3, CD45RO, CCR7, CD8, HLADR, CD86, and CD14 in a sample obtained from the subject, and administering the immune checkpoint inhibitor to the subject. In another example, the immune checkpoint inhibitor further comprising that one or more anti-cancer drugs are to be administered to the subject.

In one example, the present disclosure provides a kit for predicting the occurrence of a complete or partial response in a subject suffering from liver cancer if the subject were to receive an immune checkpoint inhibitor treatment. In another example, the present disclosure provides a kit for predicting the occurrence of a complete or partial response in a subject suffering from liver cancer if the subject is receiving an immune checkpoint inhibitor treatment. In some examples, the kit comprises at least one agent adapted to target one or more biomarkers in a sample obtained from the subject. In some examples, the at least one agent adapted to target one or more biomarkers is an antibody or antigen binding fragment thereof. In some further examples, the at least one agent adapted to target one or more biomarkers is a monoclonal antibody. In some examples, the at least one agent is for detecting an immune cell population that comprises one or more biomarkers selected from the group consisting of CXCR3, CD45RO, CCR7, CD8, HLADR, ITGAX (CD11c), and CD86, wherein the detection of the immune cell population that comprises the one or more biomarkers indicates that the treatment of the subject with an immune checkpoint inhibitor results in a complete or partial response in the subject. In some further examples, the at least one agent is for detecting an immune cell population that comprises one or more biomarkers selected from the group consisting of CXCR3, CD45RO, CCR7, CD8, HLADR, ITGAX (CD11c), and CD86, wherein the non-detection of the immune cell population that comprises one or more biomarkers indicates that the treatment of the subject with an immune checkpoint inhibitor does not result in a complete or partial response in the subject.

In one example, the present disclosure provides a kit for predicting the occurrence of one or more treatment-induced immune-related adverse events (irAEs) in a subject suffering from liver cancer if the subject were to receive an immune checkpoint inhibitor treatment. In another example, the present disclosure provides a kit for predicting the occurrence of one or more treatment-induced immune-related adverse events (irAEs) in a subject suffering from liver cancer if the subject is receiving an immune checkpoint inhibitor treatment. In some examples, the kit comprises at least one agent adapted to target one or more biomarkers in a sample obtained from the subject. In some examples, the at least one agent adapted to target one or more biomarkers is an antibody or antigen binding fragment thereof. In some further examples, the at least one agent adapted to target one or more biomarkers is a monoclonal antibody. In some examples, the at least one agent is for method comprises detecting an immune cell population that comprises one or more biomarkers selected from the group consisting of CXCR3, CD45RO, CCR7, CD8, HLADR, CD86, and CD14, wherein the detection of the immune cell population that comprises one or more biomarkers indicates that the treatment of the subject with an immune checkpoint inhibitor does not result in one or more treatment-induced immune-related adverse events (irAEs) in the subject. In some further examples, the at least one agent is for detecting an immune cell population that comprises one or more biomarkers selected from the group consisting of CXCR3, CD45RO, CCR7, CD8, HLADR, CD86, and CD14, wherein the non-detection of the immune cell population that comprises one or more biomarkers indicates that the treatment of the subject with an immune checkpoint inhibitor results in one or more treatment-induced immune-related adverse events (irAEs) in the subject.

In another example, the disclosure provides a medicament comprising an immune checkpoint inhibitor for treating liver cancer in a subject, wherein the subject has an immune cell population which comprises one or more biomarkers selected from the group consisting of CXCR3, CD45RO, CCR7, CD8, HLADR, ITGAX (CD11c), and CD86.

In another example, the disclosure provides a medicament comprising an immune checkpoint inhibitor for treating liver cancer in a subject, wherein the subject has an immune cell population which comprises one or more biomarkers selected from the group consisting of CXCR3, CD45RO, CCR7, CD8, HLADR, CD86, and CD14.

In one example, the cancer is a liver cancer. In another example, the cancer can be, but is not limited to hepatocellular carcinoma, cholangiocarcinoma, and hepatoblastoma.

In one example, the subject is a mammal. In another example, the subject is a human. In some examples, the subject is a cancer patient. In some particular examples, the subject is a liver cancer patient.

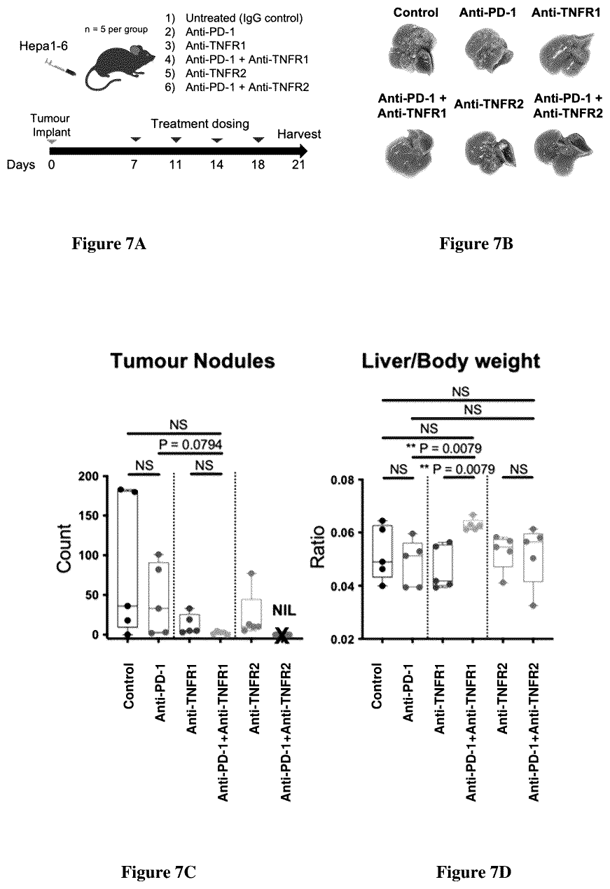

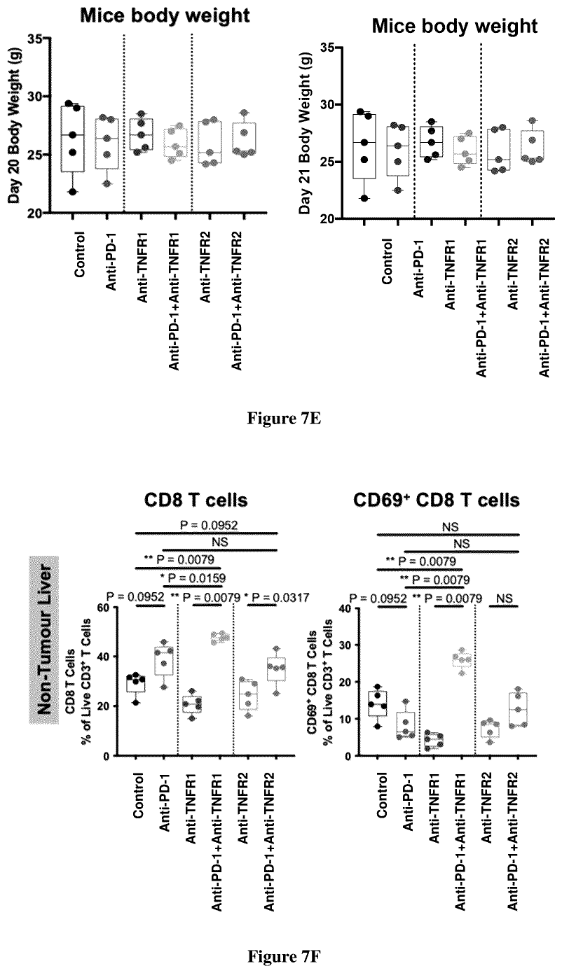

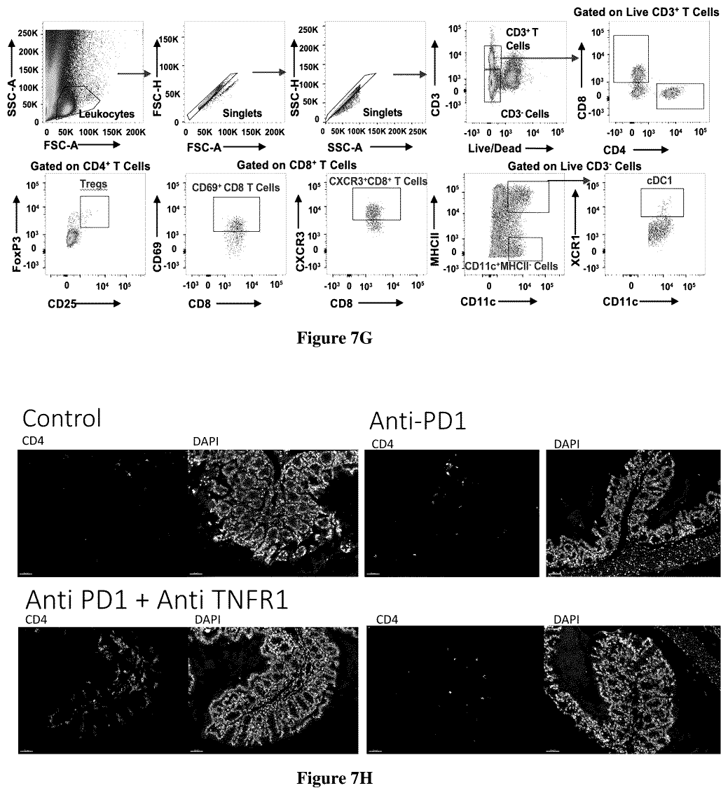

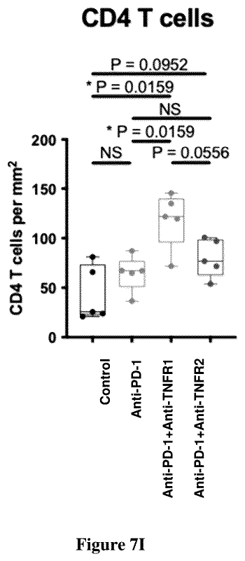

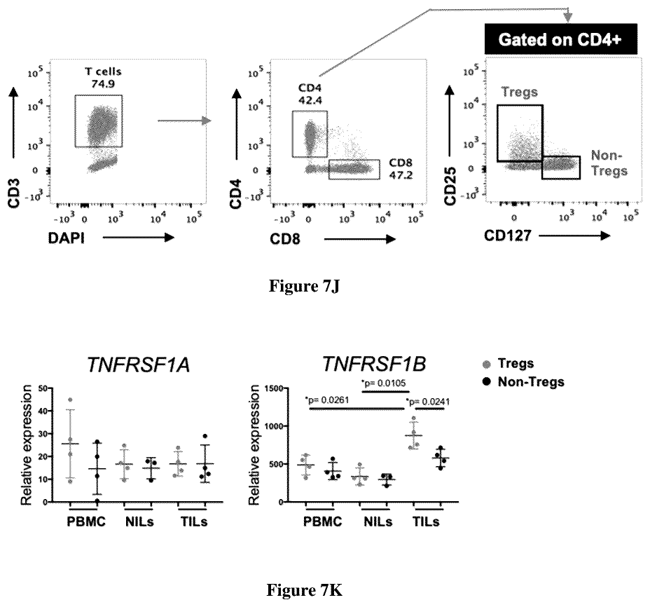

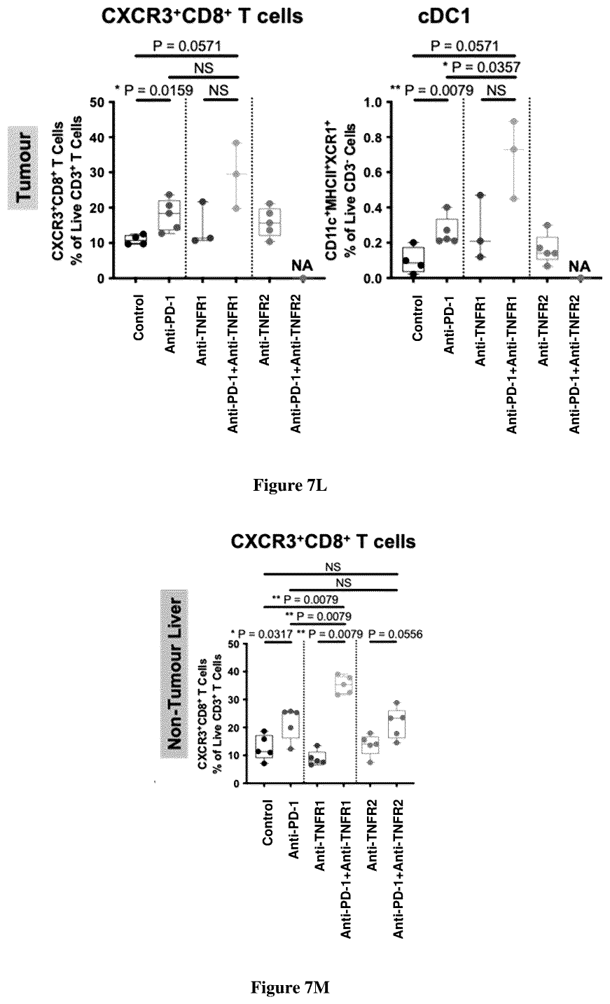

In one example, the immune checkpoint protein can be, but is not limited to PD-1, PD-L1, CTLA-4, TIGIT, LAG3, and Tim3. A person skilled in the art would be able to understand immune checkpoint proteins and identify their suitable inhibitors, without specific limitation on the inhibitory mechanism. In some examples, the immune checkpoint inhibitors are monoclonal antibodies specifically targeting and inhibiting one or more immune checkpoint proteins. In one example, the immune checkpoint inhibitor is anti-PD-1. In another example, the immune checkpoint inhibitor is anti-PD-L1. In another example, the immune checkpoint inhibitor is anti-CTLA-4. In another example, the immune checkpoint inhibitor is anti-TIGIT. In another example, the immune checkpoint inhibitor is anti-LAG3. In another example, the immune checkpoint inhibitor is anti-Tim3. In another example, the immune checkpoint inhibitor can be, but is not limited to: anti-PD-1, anti-PD-L1, anti-CTLA-4, anti-TIGIT, anti-LAG3, and anti-Tim3, and any combination thereof. As demonstrated in the mice hepatocellular carcinoma (HCC) model of FIG. 7A and FIG. 7C, mice with induced hepatocellular carcinoma (HCC) are treated with exemplary immune checkpoint inhibitor with or without anti-TNFR1 or anti-TNFR2. At Day 21, all mice receiving combination treatments showed significant reduction in tumour nodules, especially those treated with anti-PD-1+anti-TNFR2, which displayed no tumour burden.

In one example, the administering of immune checkpoint inhibitor and anti-cancer drug is simultaneous. In another example, the administering of immune checkpoint inhibitor and anti-cancer drug is separate. In another example, the immune checkpoint inhibitor is administered once every 2 weeks. In another example, the immune checkpoint inhibitor is administered once every 3 weeks. In some examples, the immune checkpoint inhibitor is administered on day 7, 11, 14, and 18 of the treatment.

In some examples, the immune checkpoint inhibitor is administered to the subject for as long as it is tolerable and beneficial for the subject. In some examples, the immune checkpoint inhibitor is administered to the subject for no more than 2 years. In some examples, the immune checkpoint inhibitor is administered to the subject for about 2-24 weeks, or about 2-8 weeks, about 7-16 weeks, about 15-24 weeks, or about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks. A person skilled in the art would be able to understand that the frequency and dosage of administration of one or more drugs to a subject are affected by factors such as body size, metabolism, gender, and disease status. Therefore, a person skilled in the art can carry out routine optimisation to determine suitable intervals for administration of the immune checkpoint inhibitor, and to determine whether such administration is beneficial or tolerable to the subject.

In one example, the method as disclosed herein, or the immune checkpoint inhibitor as disclosed herein, or the use disclosed herein further comprises one or more anti-cancer drugs. The anti-cancer drug can be a small molecule. The anti-cancer drug can be a small molecule drug, or an antibody. In some examples, the anticancer drug is a monoclonal antibody.

In one example, the one or more anti-cancer drugs is TNFR2 inhibitor. In another example, the one or more anti-cancer drugs is Notch 1 inhibitor. In another example, the one or more anti-cancer drugs is anti-VEGFA. In another example, the one or more anti-cancer drugs is anti-tyrosine kinase inhibitors (TKIs). In some further examples, the one or more anti-cancer drugs can be, but are not limited to TNFR2 inhibitor, Notch 1 inhibitor, anti-LTBR, anti-VEGFA, tyrosine kinase inhibitors (TKIs) and any combination thereof. In another example, the one or more anti-cancer drugs is an antibody against TNFR2, Notch 1, LTBR, VEGFA, tyrosine kinase (TK) and any combination thereof. A person skilled in the art is able to understand and elect suitable anti-cancer drugs available for the treatment of cancer.

In another example, the anti-cancer drug is administered once every 2 weeks. In another example, the anti-cancer drug is administered once every 3 weeks. In some examples, the immune checkpoint inhibitor is administered on day 7, 11, 14, and 18 of the treatment.

In some examples, the anti-cancer drug is administered to the subject for as long as it is tolerable and beneficial for the subject. In some examples, the anti-cancer drug is administered to the subject for no more than 2 years. In some examples, the anti-cancer drug is administered to the subject for about 2-24 weeks, or about 2-8 weeks, about 7-16 weeks, about 15-24 weeks, or about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks. A person skilled in the art would be able to understand that the frequency and dosage of administration of one or more drugs to a subject are affected by factors such as body size, metabolism, gender, and disease status. Therefore, a person skilled in the art can carry out routine optimisation to determine suitable intervals for administration of the anti-cancer drug, and to determine whether such administration is beneficial or tolerable to the subject.

In another aspect, the present disclosure provides a kit or panel of biomarkers for evaluating a complete or partial response of a subject suffering from liver cancer to a treatment with an immune checkpoint inhibitor. In one example, the kit or panel comprises at least one antibody adapted to target one or more biomarkers in a sample obtained from the subject. In one particular example, the one or more biomarkers targeted comprises at least CXCR3, CD45RO, CCR7, CD8, HLADR, ITGAX (CD11c), and CD86.

In another aspect, the present disclosure provides a kit or panel of biomarkers for evaluating treatment-induced immune-related adverse events (irAEs) of a subject suffering from liver cancer to a treatment with an immune checkpoint inhibitor. In one example, the kit or panel comprises at least one antibody adapted to target one or more biomarkers in a sample obtained from the subject. In one particular example, the one or more biomarkers targeted comprises at least CXCR3, CD45RO, CCR7, CD8, HLADR, CD14 and CD86.

In another aspect, the present disclosure provides a panel of biomarkers for evaluating complete or partial response of a subject suffering from liver cancer to a treatment with an immune checkpoint inhibitor, the panel comprising one or more biomarkers selected from the group consisting of CXCR3, CD45RO, CCR7, CD8, HLADR, ITGAX (CD11c), and CD86.

In another aspect, the present disclosure provides a panel of biomarkers for evaluating treatment-induced immune-related adverse events (irAEs) of a subject suffering from liver cancer to a treatment with an immune checkpoint inhibitor, the panel comprising one or more biomarkers selected from the group consisting of CXCR3, CD45RO, CCR7, CD8, HLADR, CD86, and CD14.

In another aspect, the present disclosure provides a panel of biomarkers for predicting a complete or partial response of a subject suffering from liver cancer, if the subject were to receive a treatment with an immune checkpoint inhibitor, the panel comprising one or more biomarkers selected from the group consisting of CXCR3, CD45RO, CCR7, CD8, HLADR, ITGAX (CD11c), and CD86.

In another aspect, the present disclosure provides a panel of biomarkers for predicting treatment-induced immune-related adverse events (irAEs) of a subject suffering from liver cancer, if the subject were to receive a treatment with an immune checkpoint inhibitor, the panel comprising one or more biomarkers selected from the group consisting of CXCR3, CD45RO, CCR7, CD8, HLADR, CD86, and CD14.

In another example, the sample is an ex vivo sample. In another example, the sample can be, but is not limited to a tissue sample or bodily fluid sample. In another example, the sample is a solid or liquid biopsy sample. In another example, the sample can be, but is not limited to cellular components of a liquid biopsy, interstitial fluid, peritoneal fluids, peripheral blood, whole blood, plasma, and serum.

The disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

It should further be appreciated that the exemplary embodiments are only examples, and are not intended to limit the scope, applicability, dimensions, or configuration of the invention in any way. Rather, the foregoing detailed description will provide those skilled in the art with a convenient road map for implementing an exemplary embodiment of the invention, it being understood that various changes may be made in the function and arrangement of elements and method of fabrication described in an exemplary embodiment without departing from the scope of the invention as set forth in the appended claims.

### Experimental Section

Immune checkpoint blockade (ICB) has achieved promising outcomes in treating cancer, including hepatocellular carcinoma (HCC). While recently reported combination immunotherapies for hepatocellular carcinoma (HCC) conferred greater objective response rates, immune-related adverse events (irAEs) increased in tandem. The present disclosure investigates the coupling mechanism of the response and immune-related adverse events (irAEs) in liver cancer patients subjected to immunotherapy and provides the mechanisms of response and/or immune-related adverse events (irAEs) in immune checkpoint blockade to predict and improve treatment outcomes.

The development of single-cell, multi-parametric technologies has provided the means to extract valuable data from limited samples, enabling in-depth characterisation of the immune landscape for mechanistic and biomarker discovery. Response to immunotherapy requires re-activation of the immunosuppressive tumour microenvironment (TME). Nonetheless, the systemic immune landscape plays an important role in the anti-tumour immune response and provides a practical and minimally-invasive source of biomarkers in the clinical setting.

In the present disclosure, deep single-cell immunoprofiling of hepatocellular carcinoma (HCC) patients treated with anti-PD-1 immune checkpoint blockade is conducted to discover immune signatures predictive of response and deciphers the mechanisms behind response versus immune-related adverse events (irAEs).

### Overall Workflow

Pre- and on-treatment peripheral blood samples (n=60) obtained from 32 hepatocellular carcinoma (HCC) patients in Singapore were analysed by cytometry by time-of-flight (CyTOF) and single-cell RNA sequencing (scRNA-seq) with flow cytometric validation in an independent Korea cohort (n=29). Mechanistic validation was conducted by bulk RNA sequencing of 20 pre- and on-treatment tumour biopsies and using a murine hepatocellular carcinoma (HCC) model treated with different immunotherapeutic combinations.

### Patient Samples

Hepatocellular carcinoma (HCC) patients receiving anti-PD-1 immune checkpoint blockade: nivolumab or pembrolizumab from the National Cancer Centre Singapore (SG cohort n=32, Table 1, real-world clinical cohort) and nivolumab from the Asan Medical Center, South Korea (KR cohort n=29, Table 2, NCT03695952), were recruited with written informed consent following each institution's Institutional-Review-Board's guidelines. Patients received intravenous nivolumab (3 mg/kg) every two weeks or pembrolizumab (200 mg) every three weeks. Blood samples were collected at baseline (both cohorts) and during treatment (SG cohort only). Treatment response was monitored and assessed according to Response Evaluation Criteria In Solid Tumors (RECIST; version 1.1) guideline and immune-related adverse events (irAEs) were assessed with National Cancer Institute Common Terminology Criteria for Adverse Events (NCI CTCAE; version4.03). Peripheral blood mononuclear cells (PBMC) were isolated using Ficoll-Paque Plus (GE Healthcare, UK) (SG Cohort) or Lymphocyte Separation Medium (Corning) (KR cohort). mRNA from pre-treatment and 1-week on-treatment tumour biopsies were obtained (n=10 patients, SG cohort).

TABLE 1
Singapore cohort demographic and analyses information

| | | | | | | | | | | | Best | | irAEs |
|---|---|---|---|---|---|---|---|---|---|---|---|---|---|
| | | | | | Tumour | Child | | | AFP | MVI | Response | Response | Status |
| Patient | Anti- | | | | Stage | Pugh | Viral | Steatohepatitis | level | (Yes/ | (RECIST | Status | (T/NT/ |
| ID | PD-1 | Age | Gender | Race | (BCLC) | Score | Status | Status | (ng/ml) | No) | 1.1) | (R/NR) | Un) |
| HCC1 | nivo | 69 | M | O | C | A6 | Hep C | NA | 48392 | Yes | PR | R | T |
| HCC2 | nivo | 84 | F | Ch | C | A5 | NV | NASH | 11.5 | No | PR | R | NT |
| HCC3 | nivo | 74 | M | Ch | C | B7 | NV | ASH | NA | Yes | SD <6 m | NR | NT |
| HCC4 | nivo | 74 | M | Ch | C | A5 | NV | Cryptogenic | 5374 | No | PD | NR | NT |
| HCC5 | nivo | 48 | M | O | B | A6 | Hep B | NA | 13.5 | Yes | PR | R | NT |
| HCC6 | nivo | 81 | M | Ch | C | A6 | Hep B | NA | 2.9 | Yes | PR | R | T |
| HCC7 | nivo | 73 | M | O | C | A5 | Hep C | NA | 62 | No | SD >6 m | R | NT |
| HCC8 | nivo | 74 | M | Ch | B | B7 | Hep B | NA | 2870 | No | PR | R | T |
| HCC9 | nivo | 66 | M | Ch | C | A6 | NV | NASH | 88.3 | No | SD <6 m | NR | T |
| HCC10 | nivo | 62 | M | Ch | C | A6 | Hep B | NA | 197 | No | PR | R | NT |
| HCC11 | nivo | 72 | M | Ch | C | A5 | Hep B | NA | 12.9 | No | SD >6 m | R | NT |
| HCC12 | nivo | 66 | M | Ch | C | A6 | Hep C | NA | 3883 | No | PD | NR | NT |
| HCC13 | nivo | 62 | M | Ch | B | A5 | NV | NASH | 16.2 | Yes | PR | R | T |
| HCC14 | nivo | 76 | M | O | B | A6 | NV | NASH | 2.7 | No | PR | R | T |
| HCC15 | nivo | 58 | F | Ch | C | A5 | Hep B | NA | 31119 | Yes | PD | NR | Un |
| HCC16 | nivo | 78 | M | Ch | C | A6 | NV | NASH | 3.4 | No | SD >6 m | R | NT |
| HCC17 | nivo | 71 | F | Ch | C | A5 | Hep B | NA | 2.3 | Yes | SD <6 m | NR | NT |
| HCC18 | nivo | 68 | M | Ch | D | B7 | Hep C | NA | >60500 | Yes | PD | NR | NA |
| HCC19 | nivo | 69 | M | Ch | C | B8 | NV | NASH | 3.1 | Yes | PD | NR | T |
| HCC20 | nivo | 69 | M | O | C | A5 | NV | NASH | 11.8 | Yes | PD | NR | Un |
| HCC21 | nivo | 66 | M | Ch | B | A6 | Hep B | NA | 69.1 | No | SD <6 m | NR | NT |
| HCC22 | nivo | 70 | M | Ch | C | A6 | Hep B | NA | 18427 | Yes | PR | R | T |
| HCC23 | nivo | 72 | M | Ch | C | A6 | NV | NASH | 15669 | Yes | PD | NR | Un |
| HCC24 | nivo | 53 | M | Ch | B | B8 | Hep B | NA | 1172 | No | PD | NR | NT |
| HCC25 | nivo | 61 | M | Ch | C | A5 | Hep B | NA | 19274 | No | PD | NR | NT |
| HCC26 | pembro | 70 | M | O | B | B7 | Hep B | NA | 641 | No | PD | NR | NT |
| HCC27 | nivo | 76 | M | O | C | A6 | Hep B | NA | 1112 | Yes | SD >6 m | R | T |
| HCC28 | nivo | 79 | M | Ch | C | B7 | NV | NASH | 7.2 | No | SD >6 m | R | T |
| HCC29 | nivo | 67 | M | Ch | A | B7 | NV | ASH | 4886 | No | PD | NR | NT |
| HCC30 | nivo | 70 | M | Ch | C | A6 | Hep B | NA | 6.3 | Yes | PD | NR | NT |
| HCC31 | pembro | 55 | M | O | C | A5 | NV | NASH | 7.6 | No | PD | NR | T |
| HCC32 | nivo | 62 | M | Ch | C | B9 | NV | Cryptogenic | 14642 | Yes | PD | NR | T |

| | | | | | | | | CyTOFT vs NT | | | |
|---|---|---|---|---|---|---|---|---|---|---|---|
| | irAE | | Tumour | | CyTOF R vs NR | | | Analyses | | | |
| | Grade (G) | EHS | Focality | Prior | Analyses | | | Tox | | | |
| Patient | and | (Yes/ | (Multi/ | Therapy | | <6 | >10 | (±Non- | | scRNA | Tissue |
| ID | Nature | No) | Uni) | Received | Baseline | Wks | Wks | 2 wks) | Tox | seq | RNAseq\hat{ } |
| HCC1 | G3 Myositis | Yes | Multi | Surg | | | | + | | | |
| HCC2 | None | Yes | Multi | Surg | | | | | + | | |
| HCC3 | G1 Itch | Yes | Multi | SIRT, | | | | | + | + | + |
| | | | | RFA | | | | | | | |
| HCC4 | G1 Transaminitis | Yes | Multi | SIRT | | | | | + | | + |
| HCC5 | None | No | Uni | SIRT | | | | | + | | |
| HCC6 | G3 Rash, | No | Uni | SIRT | +# | | + | + | | +* | + |
| | G2 Cheilitis, | | | | | | | | | | |
| | G2 Mucositis | | | | | | | | | | |
| HCC7 | G1 Itch, | No | Multi | SIRT | | | | | + | | |
| | G1 Rash | | | | | | | | | | |
| HCC8 | G2 Rash | No | Multi | SIRT | | | | + | | + | + |
| HCC9 | G2 Hyperthyroidism, | Yes | Multi | TACE, | | | | + | | + | |
| | G1 Rash | | | GEM, | | | | | | | |
| | | | | 5FU, | | | | | | | |
| | | | | SIRT | | | | | | | |
| HCC10 | None | Yes | Multi | TACE, | | +# | | | + | + | + |
| | | | | RFA | | | | | | | |
| HCC11 | None | No | Uni | TACE, | | | | | + | + | |
| | | | | SIRT | | | | | | | |
| HCC12 | None | Yes | Multi | TACE, | | +# | | | + | | |
| | | | | Sor | | | | | | | |
| HCC13 | G2 Rash, | No | Multi | Surg. | | +# | + | + | | + | + |
| | G2 Itch | | | TACE | | | | | | | |
| HCC14 | G2 Itch, | No | Multi | SIRT | | +# | + | + | | + | + |
| | G1 Fatigue, | | | | | | | | | | |
| | G1 Dysegusia, | | | | | | | | | | |
| | G1 Diarrhoea | | | | | | | | | | |
| HCC15 | G1 Fatigue, | Yes | Multi | SIRT | | | | | | | + |
| | G1 Rash, | | | | | | | | | | |
| | G1 Cough | | | | | | | | | | |
| HCC16 | G1 Itch, | No | Multi | SIRT | | +# | + | | + | | |
| | G1 Pneumonitis | | | | | | | | | | |
| HCC17 | G1 Diarrhoea | No | Multi | Surg, | +# | | + | | + | | + |
| | | | | TACE | | | | | | | |
| HCC18 | None | No | Multi | SIRT | +# | | | | | | |
| HCC19 | G2 Rash | No | Multi | None | | +# | | + | | + | |
| HCC20 | None | No | Multi | None | +# | | | | | | |
| HCC21 | None | Yes | Multi | SIRT | | | | | + | | + |
| HCC22 | G3 Aspartate | No | Multi | None | +# | | + | + | | | |
| | aminotransferase | | | | | | | | | | |
| | elevation, | | | | | | | | | | |
| | G3 Deranged | | | | | | | | | | |
| | liver function | | | | | | | | | | |
| | test, | | | | | | | | | | |
| | G2 Rash, | | | | | | | | | | |
| | G2 Dermatitis | | | | | | | | | | |
| HCC23 | None | Yes | Multi | SIRT | +# | | | | | | |
| HCC24 | G1 Rash | No | Multi | Surg, | + | | | | | | |
| | | | | Tace | | | | | | | |
| HCC25 | None | Yes | Multi | Surg, | + | | | | | | |
| | | | | RFA, | | | | | | | |
| | | | | EBRT, | | | | | | | |
| | | | | Lenv | | | | | | | |
| HCC26 | None | Yes | Multi | TACE, | + | | | | | | |
| | | | | Sor | | | | | | | |
| HCC27 | G2 Itch, | No | Multi | None | + | | + | | | | |
| | G2 Rash | | | | | | | | | | |
| | G2 Liver enzyme | | | | | | | | | | |
| HCC28 | derangement, | Yes | Uni | None | | + | + | | | | |
| | G2 Adrenal | | | | | | | | | | |
| | insufficiency, | | | | | | | | | | |
| | G1 Rash | | | | | | | | | | |
| HCC29 | None | No | Uni | None | + | | | | | | |
| HCC30 | None | No | Multi | None | + | | | | | | |
| HCC31 | G2 Psoriasis | Yes | Multi | Surg, | + | | + | + | | | |
| | flare | | | Lenv | | | | | | | |
| HCC32 | G2 Psoriasis | No | Multi | None | + | | | | | | |
| | flare | | | | | | | | | | |
| | | | | | | | | | | | Total |
| | | | | | | | | | | | n: 80 |

Anti-PD-1: nivo—nivolumab; pembro—pembrolizumab; Gender: M—Male and F—Female; Race: O—Others and Ch—Chinese;
Tumour Stage: BCLC—Barcelona Clinic Liver Cancer staging system; Viral status: Hep B/C—Hepatitis B/C virus carrier; NV—Non-viral history;
Steatohepatitis Status: NASH—Non-alcoholic steatohepatitis; ASH—Alcoholic steatohepatitis; NA—Not Applicable; AFP: Alpha-fetoprotein; MVI: Macrovascular invasion;
RECIST 1.1: Response Evaluation Criteria in Solid Tumours Version 1.1. PR—Partial Response; SD—Stable Disease; PD—Progressive Disease;
Response Status: R—Responder (Partial response/Stable disease >6 months); NR—Non-Responder (Progressive disease/Stable disease <6 months);
irAEs Status: T—Tox (Patient experienced irAEs ≥G2); NT—Non-Tox (Patient experienced G1/no irAEs); Un—irAEs status for patient was undetermined;
EHS: Extrahepatic Spread; Tumour Focality: Multi—Multifocal; Uni—Unifocal;
Prior Therapy Received: Surg—Surgery; SIRT—Selective Internal Radiation Therapy; RFA—Radiofrequency Ablation; TACE—Transarterial Chemoembolization; GEM—Gemcitabine; 5FU—Fluorouracil; Sor—Sorafenib; EBRT—External Beam Radiation Therapy; Lenv—Lenvatinib
+: Samples used in the respective experiments
#Sample used in unsupervised CyTOF analysis
+*: Patient had both pre- and on-treatment samples
\hat{ }Pre-treatment and 1 week on-treatment samples were used

TABLE 2
Korea cohort demographic information

| | | | | Tumour | Child | | | AFP | MVI |
|---|---|---|---|---|---|---|---|---|---|
| | | | | Stage | Pugh | Viral | Steatohepatitis | level | (Yes/ |
| Patient ID | Age | Gender | Race | (BCLC) | Score | Status | Status | (ng/ml) | No) |
| HCC33 | 58 | M | Asian | C | B7 | Hep B | NA | 15.5 | Yes |
| HCC34 | 36 | F | Asian | C | A5 | Hep B | NA | 29749 | No |
| HCC35 | 63 | M | Asian | C | A6 | Hep B | NA | 549.7 | Yes |
| HCC36 | 58 | M | Asian | C | A5 | Hep B | NA | 8740.6 | No |
| HCC37 | 58 | M | Asian | C | A5 | Hep B | NA | 1660 | Yes |
| HCC38 | 56 | M | Asian | C | A6 | Hep B | NA | 24771 | Yes |
| HCC39 | 66 | M | Asian | C | B7 | Hep B | NA | 286 | No |
| HCC40 | 59 | M | Asian | C | A5 | NV | NASH | 124.2 | No |
| HCC41 | 49 | M | Asian | C | A6 | Hep B | NA | 140 | Yes |
| HCC42 | 64 | M | Asian | C | A5 | NV | ASH | 10499.8 | No |
| HCC43 | 76 | M | Asian | C | A6 | Hep B | NA | 2.9 | Yes |
| HCC44 | 73 | M | Asian | C | A5 | Hep B | NA | NA | Yes |
| HCC45 | 59 | M | Asian | C | A5 | Hep B | NA | 43.5 | No |
| HCC46 | 61 | M | Asian | C | A5 | NV | NASH | 5.3 | Yes |
| HCC47 | 62 | M | Asian | C | B9 | NV | ASH | 62418 | Yes |
| HCC48 | 75 | M | Asian | C | A6 | Hep B | NA | 3 | Yes |
| HCC49 | 58 | M | Asian | C | A5 | Hep B | NA | 128 | Yes |
| HCC50 | 74 | M | Asian | C | A6 | Hep B | NA | 40.4 | No |
| HCC51 | 67 | M | Asian | C | A5 | Hep C | NA | 214 | No |
| HCC52 | 49 | M | Asian | C | A5 | Hep B | NA | 48223 | Yes |
| HCC53 | 55 | M | Asian | C | A6 | Hep B | NA | 3003 | Yes |
| HCC54 | 75 | M | Asian | C | A6 | NV | NA | 72 | No |
| HCC55 | 61 | M | Asian | C | A6 | Hep B | NA | 62 | No |
| HCC56 | 66 | M | Asian | C | B8 | Hep B | NA | 77 | No |
| HCC57 | 52 | M | Asian | C | A6 | Hep B | NA | 0 | Yes |
| HCC58 | 54 | M | Asian | C | A6 | Hep B | NA | 3 | Yes |
| HCC59 | 57 | M | Asian | C | A6 | Hep B | NA | 3 | No |
| HCC60 | 41 | M | Asian | C | A6 | Hep B | NA | 2 | No |
| HCC61 | 67 | M | Asian | C | A5 | Hep C | NA | 2584 | No |

| | Best | | | | | Tumour | |
|---|---|---|---|---|---|---|---|
| | Response | Response | irAEs | Nature | EHS | Focality | Prior |
| | (RECIST | Status | Status | of | (Yes/ | (Multi/ | Therapy |
| Patient ID | 1.1) | (R/NR) | (T/NT) | irAEs | No) | Uni) | Received |
| HCC33 | SD >6 m | R | T | G2 Rash | No | Multi | Sor |
| HCC34 | PD | NR | NT | None | Yes | Multi | Surg, TACE, |
| | | | | | | | RT, Sor |
| HCC35 | PD | NR | NT | None | Yes | Multi | Sor |
| HCC36 | PD | NR | NT | None | Yes | Uni | TACE, RT, |
| | | | | | | | Sor |
| HCC37 | PD | NR | NT | None | Yes | Multi | TACE, RT, |
| | | | | | | | Sor |
| HCC38 | SD >6 m | R | NT | None | Yes | Multi | TACE, RFA, |
| | | | | | | | RT Sor |
| HCC39 | PR | R | NT | None | Yes | Uni | Surg, RT, Sor |
| HCC40 | SD <6 m | NR | NT | None | Yes | Multi | RT, Sor |
| HCC41 | PD | NR | NT | None | Yes | Multi | Sor |
| HCC42 | PD | NR | T | G2 Rash | Yes | Multi | TACE, RT, |
| | | | | | | | Sor |
| HCC43 | PD | NR | NT | None | Yes | Multi | Sor |
| HCC44 | PR | R | NT | None | Yes | Uni | Surg, TACE, |
| | | | | | | | Sor |
| HCC45 | PD | NR | NT | None | Yes | Uni | RT, Sor |
| HCC46 | SD <6 m | NR | NT | None | Yes | Uni | Surg, TACE, |
| | | | | | | | RT, Sor |
| HCC47 | PD | NR | NT | None | Yes | Multi | TACE, RFA, |
| | | | | | | | RT Sor |
| HCC48 | PD | NR | NT | None | Yes | Multi | Surg, TACE, |
| | | | | | | | RFA, RT, Sor |
| HCC49 | PR | R | T | G3 Hepatitis | Yes | Uni | Surg, TACE, |
| | | | | | | | RFA, RT, Sor |
| HCC50 | SD >6 m | R | NT | None | No | Uni | TACE, RT, |
| | | | | | | | Sor |
| HCC51 | PD | NR | NT | None | Yes | Uni | TACE, RFA, |
| | | | | | | | RT Sor |
| HCC52 | PR | R | T | G3 Hepatitis | Yes | Uni | Surg, Sor |
| HCC53 | PR | R | NT | NA | Yes | Multi | TACE, RT, |
| | | | | | | | Sor |
| HCC54 | NA | NR | NT | NA | Yes | Multi | Surg, TACE, |
| | | | | | | | RT, Sor |
| HCC55 | SD <6 m | NR | NT | NA | Yes | Multi | TACE, Sor |
| HCC56 | PD | NR | NT | NA | Yes | Multi | Surg, Sor |
| HCC57 | PD | NR | NT | NA | Yes | Multi | Surg, TACE, |
| | | | | | | | RT, Sor |
| HCC58 | PR | R | NT | NA | Yes | Multi | RT, Sor |
| HCC59 | PD | NR | NT | NA | Yes | Multi | TACE, RT, |
| | | | | | | | Sor |
| HCC60 | PD | NR | NT | NA | Yes | Multi | Sor |
| HCC61 | PD | NR | NT | NA | Yes | Multi | TACE, RFA, |
| | | | | | | | RT, Sor |

Gender: M—Male and F—Female
Tumour Stage: BCLC—Barcelona Clinic Liver Cancer staging system
Viral status: Hep B/C—Hepatitis B/C virus carrier; NV—Non-viral history
Steatohepatitis Status: NASH—Non-alcoholic steatohepatitis; ASH—Alcoholic steatohepatitis; NA—Not Applicable
AFP: Alpha-fetoprotein
MVI: Macrovascular invasion
RECIST 1.1: Response Evaluation Criteria in Solid Tumours Version 1.1. PR—Partial Response; SD>/<6 m—Stable Disease>/<6 months; PD—Progressive Disease; NA—Not Applicable
Response Status: R—Responder (Partial response/Stable disease >6 months); NR—Non-Responder (Progressive disease/Stable disease <6 months)
irAEs Status: T—Tox (Patient experienced irAEs G2 and above); NT—Non-Tox (Patient experienced G1/no irAEs)
EHS: Extrahepatic Spread
Tumour Focality: Multi—Multifocal; Uni—Unifocal
Prior-Therapy Received: Surg—Surgery; RT—Radiation Therapy; RFA—Radiofrequency Ablation; TACE—Transarterial Chemoembolization; Sor—Sorafenib

### Hepatocellular Carcinoma (HCC) Model

Male C57BL/6 mice (aged 6-8 weeks; InVivos, Singapore), housed in pathogen-free conditions according to guidelines of Institutional Laboratory Animal Care and Use Committee of the National University of Singapore, were inoculated with 1×10⁶ Hepa1-6 murine hepatoma cells via hydrodynamic tail-vein injection. From day−7, tumour-bearing mice were injected intraperitoneally on day 7, day 11, day 14 and day 18 with anti-PD-1 (RMP1-14, 250 g/mouse), anti-TNFR1 (55R-170, 250 g/mouse), anti-TNFR2 (TR75-54.7, 500 g/mouse), alone or in combination (anti-PD1+anti-TNFR1, anti-PD1+anti-TNFR2), Armenian hamster IgG (PIP, 500 g/mouse) and rat IgG2a (1-1, 250 g/mouse) (all from ichorbio, UK). On Day−21, mice were euthanized by CO₂ asphyxiation and the numbers of liver tumour nodules and liver weights were recorded. Infiltrating leucocytes from tumour and non-tumour liver tissue were isolated for flow cytometry analysis. Mouse colons were flushed and collected for formalin-fixed paraffin embedding (FFPE) processing using the Swiss-rolling method.

### Cytometry by Time-of-Flight (CyTOF)

CyTOF staining was performed with a panel of 39 antibodies (Table 3) and analysed using a Helios mass cytometer (Fluidigm, USA). Method of performing CyTOF staining are known in the art. Data were down sampled to 10,000 viable CD45⁺ cells for in-house developed Extended Poly-dimensional Immunome Characterisation. Clustering was performed with the FlowSOM algorithm, dimension reduction by tSNE, and visualisation with the R shiny app ‘SciAtlasMiner’. Enriched clusters were identified by two-tailed Mann-Whitney U (MWU) test and validated with manual gating using FlowJo (V.10.5.2; FlowJo, USA).

TABLE 3
CyTOF antibody panel

| Antigen/Clone | Company | Cataloge No. |
|---|---|---|
| Anti-Human CD45-Y89 (Clone: H130) | Fluidigm | Cat# 3089003B |
| Anti-Human CD45 (Purified) (Clone: H130) | BioLegend | Cat# 304002 |
| Anti-Human CD14 (Q-Dot 800) (Clone: TUK4) | BioLegend | Cat# Q10064 |
| Anti-Human HLA-DR (Purified) (Clone: L234) | BioLegend | Cat# 307602 |
| Anti-Human CD19 (Purified) (Clone: HIB19) | BioLegend | Cat# 302202 |
| Anti-Human CD45RO (Purified) (Clone: UCHL1) | BioLegend | Cat# 304202 |
| Anti-Human CD3 (Purified) (Clone: UCHT1) | BioLegend | Cat# 300402 |
| Anti-Human CD8 (Purified) (Clone: SK1) | BioLegend | Cat# 344702 |
| Anti-Human IL4 (Purified) (Clone: 8D4-8) | BioLegend | Cat# 500707 |
| Anti-Human IgD (Purified) (Clone: IA6-2) | BioLegend | Cat# 348202 |
| Anti-Human PD-1 (Purified) (Clone: EH12.2H7) | BioLegend | Cat# 329902 |
| Anti-Human CD4 (Purified) (Clone: SK3) | BioLegend | Cat# 344602 |
| Anti-Human KI67 (Purified) (Clone: B56) | BD Bioscience | Cat# 556003 |
| Anti-Human CD95 (Purified) (Clone: DX2) | BioLegend | Cat# 305602 |
| Anti-Human CD161 (Purified) (Clone: HP-3G10) | BioLegend | Cat# 339902 |
| Anti-Human TNFα (Purified) (Clone: MAB11) | BioLegend | Cat# 502902 |
| Anti-Human CCR7 (Purified) (Clone: G043H7) | BioLegend | Cat# 353202 |
| Anti-Human TIM-3 (Purified) (Clone: F38-2E2) | BioLegend | Cat# 345002 |
| Anti-Human CD152 (Purified) (Clone: BN13) | BD Bioscience | Cat# 555850 |
| Anti-Human CXCR6 (Purified) (Clone: K041E65) | BioLegend | Cat# 356002 |
| Anti-Human CD40 (Purified) (Clone: 5C3) | BioLegend | Cat# 334302 |
| Anti-Human CD38 (Purified) (Clone: HIT2) | BioLegend | Cat# 303502 |
| Anti-Human CD11C (Purified) (Clone: BU15) | BioLegend | Cat# 337221 |
| Anti-Human IgM (Purified) (Clone: MHM-88) | BioLegend | Cat# 314502 |
| Anti-Human CXCR5 (Purified) (Clone: RF8B2) | BD Bioscience | Cat# 552032 |
| Anti-Human CD56 (Purified) (Clone: NCAM16.2) | BD Bioscience | Cat# 559043 |
| Anti-Human CXCR3 (Purified) (Clone: G025H7) | BioLegend | Cat# 353702 |
| Anti-Human CD32B (Purified) (Clone: EP888Y) | Abcam | Cat# ab45143 |
| Anti-Human FOXP3 (Purified) (Clone: PCH101) | eBioscience | Cat# 14-4776-82 |
| Anti-Human CD24 (Purified) (Clone: ML5) | BioLegend | Cat# 311102 |
| Anti-Human CD86 (Purified) (Clone: IT2.2) | BioLegend | Cat# 305402 |
| Anti-Human IFNγ (Purified) (Clone: B27) | BioLegend | Cat# 506502 |
| Anti-Human IL17A (Purified) (Clone: BL168) | BioLegend | Cat# 512302 |
| Anti-Human CD21 (Purified) (Clone: BU32) | BioLegend | Cat# 354902 |
| Anti-Human CXCR4 (Purified) (Clone: 12G5) | BioLegend | Cat# 306502 |
| Anti-Human IgG-Fc (Purified) (Clone: M1310G05) | BioLegend | Cat# 410901 |
| Anti-Human CCR5 (Purified) (Clone: NP-6G4) | Abcam | Cat# ab115738 |
| Anti-Human Vα7.2 (Purified) (Clone: 3C10) | BioLegend | Cat# 351702 |
| Anti-Human BAFF-R (Purified) (Clone: 11C1) | BioLegend | Cat# 316902 |
| Anti-Human CD16 (Purified) (Clone: 3G8) | Fluidigm | Cat# 3209002B |

### Flow Cytometry

Baseline PBMC samples from 29 patients (KR cohort) were stained with 14 antibodies (Table 4) and analysed using a BD LSR II cytometer. For TNF ligand/receptors validation, 16 on-treatment peripheral blood mononuclear cells (PBMCs) (SG cohort) were stained with 12 antibodies (Table 4). Immune cells from mouse samples were stained with 10 antibodies (Table 5). The Intracellular Fixation/Permeabilization Buffer Set (eBioscience) was used for intracellular staining. Data were acquired using BD LSRFortessa X-20 flow cytometer. For immune stimulation, PMA/Ionocymin (Sigma) was added for 6 h with Brefeldin A/Monesin (eBiosience) added at the last 4 h of incubation. All data analysis was done using FlowJo V.10.5.2. All data analysis was conducted using FlowJo V.10.5.2.

TABLE 4
Anti-human Flow cytometry antibodies

| | | | | |
|---|---|---|---|---|
| Antigen | Fluorophore | Clone | Company | Catalog number |

For Biomarkers analysis (KR cohort)

| | | | | |
|---|---|---|---|---|
| CD8 | BV421 | SK1 | BioLegend | Cat# 344748 |
| CD11c | BV510 | BLY6 | BD Biosciences | Cat# 563026 |
| CD4 | BV605 | RPA-T4 | BD Biosciences | Cat# 562658 |
| CD3 | BV650 | UCHT1 | BD Biosciences | Cat# 563852 |
| CD86 | BV711 | 2331(FUN-1) | BD Biosciences | Cat# 563158 |
| HLA-DR | BV785 | L243 | BioLegend | Cat# 307642 |
| CD45RO | BB515 | UCHL1 | BD Biosciences | Cat# 564529 |
| CD152/CTLA-4 (i) | PerCP-eFluor710 | 14D3 | eBioscience | Cat# 46-1529-42 |
| FoxP3 (i) | PE | PCH101 | eBioscience | Cat# 12-4776-42 |
| CD14 | PE-CF594 | MϕP9 | BD Biosciences | Cat# 562335 |
| CD56 | PE-Cy7 | B159 | BE Biosciences | Cat# 557747 |
| CXCR3 | APC | G025H7 | BioLegend | Cat# 353708 |
| CD45 | Alexa Fluor 700 | 30-F11 | eBioscience | Cat# 56-9459-42 |

For Cell sorting

| | | | | |
|---|---|---|---|---|
| CD127 | BV510 | A019D5 | Biolegend | Cat#351332 |
| CD25 | PE-Cy7 | BC96 | Biolegend | Cat#302611 |
| CD3 | FITC | UCHT1 | Biolegend | Cat#300406 |
| CD4 | BV605 | OKT4 | Biolegend | Cat#317438 |
| CD45 | APC-Cy7 | H130 | Biolegend | Cat#304014 |
| CD8 | PerCP-Cy5.5 | RPA-T8 | Biolegend | Cat#301032 |

For TNF ligand/receptors validation (SG cohort)

| | | | | |
|---|---|---|---|---|
| CD11c | AF488 | 3.9 | BioLegend | Cat#301617 |
| CD14 | BUV737 | M5E2 | BD Biosciences | Cat#564444 |
| CD3 | BUV395 | UCHT1 | BD Biosciences | Cat#563546 |
| CD4 | BV785 | OKT4 | BioLegend | Cat#317442 |
| CD56 | BV605 | HCD56 | BioLegend | Cat#318333 |
| HLA-DR | PerCP-Cy5.5 | L243 | BioLegend | Cat#307629 |
| TNFR1 | APC | W15099A | BioLegend | Cat#369905 |
| CD45RO | BV510 | UCHL1 | BioLegend | Cat#304246 |
| TNFR2 | PE | 3G7A02 | BioLegend | Cat#358403 |
| CD183 (CXCR3) | BV421 | G025H7 | BioLegend | Cat#353716 |
| CD8a | BV711 | RPA-T8 | BioLegend | Cat#301043 |
| TNFa (i) | PE/Cy7 | MAb11 | BioLegend | Cat#502930 |

(i) For intracellular staining: eBioscience ™ Intracellular Fixation & Permeabilization Buffer Set (cat#88-8824-00) was used.

TABLE 5
Mouse Flow cytometry antibodies

| Antigen | Fluorophore | Clone | Company | Catalog number |
|---|---|---|---|---|
| CD16/CD32 | NA | 2.4G2 | BD Biosciences | Cat# 553142 |
| CD3e | PerCP/Cy5.5 | 145-2C11 | eBioscience | Cat# 45-0031-82 |
| CD4 | Pacific blue | GK1.5 | BioLegend | Cat# 100428 |
| CD8a | V500 | 53-6.7 | BD Biosciences | Cat# 560776 |
| CD25 | APC | 3C7 | BioLegend | Cat# 101910 |
| CXCR3 | PE/Cy7 | CXCR3-173 | BioLegend | Cat# 126516 |
| MHCII | PE | M5/114.15.2 | BioLegend | Cat# 107608 |
| CD11c | AF700 | N418 | BioLegend | Cat# 117320 |
| XCR1 | BV785 | ZET | BioLegend | Cat# 148225 |
| FoxP3 (i) | AF488 | MF-14 | BioLegend | Cat# 126406 |
| CD69 | APC/Cy7 | H1.2F3 | BioLegend | Cat# 104526 |

(i) For intracellular staining: eBioscience ™ Intracellular Fixation & Permeabilization Buffer Set (cat#88-8824-00) was used.

Single-Cell RNA Sequencing (scRNA-Seq)

scRNA-seq was performed on 10 peripheral blood mononuclear cell (PBMC) samples consisting of nine on-treatment samples (6 Res versus 3 Non-Res; 5 Tox versus 4 Non-Tox) and one matched pre-treatment sample (HCC6; Res/Tox) (Table 1). The 5′ gene expression (GEx) libraries were prepared using the 10× Genomics platform for indexed paired-end sequencing of 2×150 base pairs on an Illumina HiSeq 4000 system at 20,000 read pairs per cell. Reads were aligned to the human GRCh38 reference genome and quantified using cellranger count (10× Genomics, v3.0.2). Data repository ID: EGAS00001004843. Cells with <200 genes and >10% mitochondrial RNA were filtered, followed by analyses using Seurat (v3.0) pipelines. A total of 29 cell clusters were annotated based on the expression of known cell lineage-specific genes (Table 6).

Functional pathway analysis was conducted using Database for Annotation, Visualization and Integrated Discovery (DAVID) v6.8. CellPhoneDB 2.0 was used to analyse ligand-receptor expression and predict cell-cell communications of CXCR3-expressing CD8 T cells using default parameters.

TABLE 6
Top 50 differentially-enriched genes (DEGs) for 29 scRNA seq clusters

| | | Cluster 2 | Cluster 3 | | Cluster 5 | Cluster 6 | |
|---|---|---|---|---|---|---|---|
| Cluster 0 | Cluster 1 | CD14_2- | CD8_Eff- | Cluster 4 | NK- | CD4- | Cluster 7 |
| CD4_Naive | CD14-1 | THBD | IFNG | CD4_Th2 | CD16 | LTB | CD16 |
| TCF7 | S100A8 | CDKN1A | CD8A | LTB | GNLY | CCR7 | IFITM3 |
| LTB | S100A9 | IER3 | GZMH | IL7R | CCL4 | RGCC | LST1 |
| IL7R | CCL3L1 | LYZ | CCL5 | IL32 | GZMB | CREM | FCGR3A |
| LEF1 | S100A12 | S100A8 | NKG7 | AQP3 | NKG7 | ZNF331 | MS4A7 |
| MAL | FCN1 | PPIF | FGFBP2 | TNFRSF4 | PRF1 | SARAF | SERPINA1 |
| CCR7 | LYZ | MAFB | CD8B | LMNA | CTSW | LTB | TIMP1 |
| GIMAP7 | CCL3 | PLAUR | CST7 | FLT3LG | SPON2 | IL7R | C1QA |
| LDHB | CD14 | S100A9 | CCLA | GATA3 | CST7 | AREG | AIF1 |
| EEF1B2 | VCAN | DUSP6 | GZMA | LDHB | CCL5 | GPR183 | PSAP |
| FLT3LG | HSPA1A | S100A12 | IL32 | SPOCK2 | FGFBP2 | YPEL5 | COTL1 |
| NOSIP | HSPA1B | CYP1B1 | CD3D | NPDC1 | CCL4L2 | LEPROTL1 | SMIM25 |
| RPS5 | MS4A6A | IL1B | GZMM | CD3D | GZMA | NPM1 | FCER1G |
| RPS12 | CTSS | S100A11 | CD3G | CD3E | CD247 | MAL | RHOC |
| RPS25 | CST3 | VCAN | TRBV28 | ARHGAP15 | KLRD1 | TNFAIP3 | LILRB2 |
| EEF1G | SERPINA1 | CST3 | GZMK | CD52 | HOPX | LEF1 | SAT1 |
| CAMK4 | TMEM176B | S100A10 | KLRG1 | MAL | GZMH | LDHB | HMOX1 |
| RPS3A | TYROBP | FCN1 | CD3E | RGCC | CLIC3 | SLC2A3 | WARS |
| RPS27 | CSTA | S100A6 | IFNG | ITGB1 | FCGR3A | CSRNP1 | CSF1R |
| RPS6 | CFD | TYROBP | DUSP2 | CD5 | IFNG | RHOH | C5AR1 |
| RPL32 | AIF1 | LGALS3 | C12orf75 | CORO1B | KLRB1 | CXCR4 | CD68 |
| PIK3IP1 | CD68 | FTH1 | GZMB | ITM2A | TRBC1 | HIST1H4C | LILRB1 |
| RPL3 | FCER1G | THBD | CD52 | CRIP2 | CMC1 | CD69 | LYN |
| CISH | TKT | GRN | LINC02446 | GIMAP7 | IL2RB | ABLIM1 | HES4 |
| RPL30 | GRN | CSTA | GNLY | GSTK1 | ADGRG1 | BTG1 | FTL |
| RPL5 | PLBD1 | NAMPT | LAIR2 | PBXIP1 | KLRF1 | GTF2B | C1QB |
| MYC | TMEM176A | VIM | TUBA4A | ICOS | CD7 | ICOS | LYPD2 |
| TSHZ2 | MNDA | NINJ1 | PTPRCAP | TRAT1 | GZMM | SBDS | PECAM1 |
| RPS21 | FTL | FTL | SYNE2 | LIME1 | IFITM1 | EEF1G | IFITM2 |
| TRAT1 | KLF4 | ANPEP | ADGRG1 | RPSA | APMAP | RPS25 | PAPSS2 |
| RPL10A | LGALS2 | TYMP | CTSW | CD6 | MATK | EEF1B2 | CST3 |
| RPL9 | TSPO | PHLDA2 | HCST | HINT1 | S1PR5 | RPL3 | HLA-DPA1 |
| RPS18 | FOS | CD14 | PRF1 | IFITM1 | XCL2 | ZFP36L2 | LRRC25 |
| LINC00861 | FPR1 | CTSS | MATK | LAT | LAIR2 | RPS5 | S100A11 |
| RPS14 | SPI1 | SPI1 | CD2 | CD2 | KLRC3 | PTGER4 | CFD |
| RPS29 | S100A6 | CFD | CCL4L2 | TRADD | SH2D1B | RPS12 | SPI1 |
| RPSA | IGSF6 | APLP2 | CD6 | BIRC3 | TTC38 | TRAT1 | FTH1 |
| NOP53 | TALDO1 | SLC11A1 | IFITM1 | RPS18 | XCL1 | S1PR1 | CALHM6 |
| RPS27A | SULT1A1 | CD300E | CD99 | INPP4B | MYOM2 | SOD1 | PILRA |
| RPL13 | LGALS3 | PTPRE | TRGV2 | TTC39C | GNG2 | RPS18 | PLAUR |
| PIM1 | CFP | TKT | HLA-B | RCAN3 | IFITM2 | RPS6 | SIGLEC10 |
| C12orf57 | CPVL | TIMP1 | SYNE1 | S1PR1 | FCRL6 | FLT3LG | CXCL16 |
| RPL36 | FCGRT | AIF1 | TGFBR3 | ZFP36L2 | C12orf75 | G3BP2 | BCL2A1 |
| PIM2 | NEAT1 | SOD2 | SAMD3 | LEPROTL1 | LITAF | ARID5A | CTSS |
| RPL14 | PSAP | RNF130 | LITAF | AES | HCST | RPSA | MTSS1 |
| RPS3 | CYBB | NEAT1 | FCRL6 | OPTN | PRSS23 | RPL5 | CUX1 |
| RPL34 | NCF2 | TSPO | RARRES3 | PAG1 | SYNE1 | EEF1A1 | NPC2 |

| | | | | | | Cluster 14 | Cluster 15 |
|---|---|---|---|---|---|---|---|
| Cluster 8 | Cluster 9 | Cluster 10 | Cluster 11 | Cluster 12 | Cluster 13 | CD14_4- | CD14_5- |
| B cell_Naive | CD8_Naive | cDC2 | CD14-3 | B cell_Imm | NKT | CD36 | CXCL8 |
| CD79A | LINC02446 | HLA-DQA1 | MED29 | MS4A1 | TRDV2 | S100A9 | CXCL8 |
| MS4A1 | CD8B | HLA-DPB1 | AC091271.1 | CD79A | TRGV9 | MNDA | IL1B |
| CD74 | CCR7 | CLEC10A | ATP2B1-AS1 | IGHM | NKG7 | S100A12 | CCL3 |
| HLA-DOA1 | NELL2 | HLA-DRA | ILF3-DT | CD79B | TRDV1 | VCAN | CCL3L1 |
| IGHM | CD8A | HLA-DQB1 | HSPA1B | TCL1A | KLRB1 | TMEM176B | GOS2 |
| HLA-DRA | LEF1 | HLA-DRB1 | HSPA1A | IGKV3-20 | CCL5 | S100A8 | NFKBIZ |
| BANK1 | TCF7 | HLA-DPA1 | COQ7 | FCER2 | CST7 | FCN1 | SOD2 |
| HLA-DPB1 | NOSIP | FCER1A | NEAT1 | FAM129C | DUSP2 | CD14 | HSPA1A |
| LINC00926 | IL7R | CD74 | AF213884.3 | CD74 | GZMA | LYZ | IER3 |
| HLA-D0B1 | RHOH | CST3 | EPS8 | RALGPS2 | GZMH | TMEM176A | S100A8 |
| TCL1A | LDHB | CD1C | AL645728.1 | LINC00926 | KLRG1 | MS4A6A | PLAUR |
| CD37 | CD7 | LMNA | C1orf43 | BANK1 | CMC1 | NCF1 | NFKBIA |
| FAM129C | RPS5 | HLA-DMA | CSF3R | HLA-DQA1 | GZMM | CTSS | S100A12 |
| CD79B | LTB | CDKN1A | STAB1 | IGHD | IL32 | FCER1G | MAFB |
| FCER2 | EEF1B2 | INSIG1 | TSPYL2 | CD37 | CD3D | LGALS2 | RETN |
| RALGPS2 | RPS12 | HLA-DRB5 | SAT1 | HLA-DPB1 | TRBC1 | SERPINA1 | NEAT1 |
| HLA-DRB1 | RGCC | CPVL | VPS9D1 | HLA-DRA | PRF1 | SPI1 | NAMPT |
| CXCR4 | MYC | KLF4 | AL118516.1 | PLPP5 | CTSW | PSAP | BCL2A1 |
| CD83 | RPS6 | PHLDA2 | HSPA6 | VPREB3 | GZMB | TYMP | EGR1 |
| HLA-DPA1 | LEPROTL1 | LYZ | POMZP3 | HVCN1 | TUBA4A | TYROBP | TNFAIP6 |
| AFF3 | RPS18 | ANXA2 | CD300LB | HLA-DQB1 | TRDC | GRN | PHLDA2 |
| IGHD | FLT3LG | HLA-DQA2 | Z93241.1 | MEF2C | KLRD1 | TSPO | HSPA1B |
| CXCR5 | EEF1G | CEBPD | HSPH1 | AFF3 | CREM | CD36 | NCF1 |
| PLPP5 | G3BP2 | PPIF | ATP6V1E1 | HLA-DPA1 | CD3G | TALDO1 | CXCL2 |
| VPREB3 | RPLI0A | GPR183 | AC087239.1 | IGLL5 | LAG3 | CYBB | MNDA |
| BACH2 | NUCB2 | IER3 | TYMP | CD22 | NCR3 | CST3 | S100A11 |
| MEF2C | RPSA | HLA-DMB | CLEC7A | FCMR | HOPX | CAPG | DUSP1 |
| CD69 | RPL13 | GSN | UBXN11 | FCRLA | MATK | CD68 | CD83 |
| BLK | ABLIM1 | HBEGF | NR4A1 | FCRL1 | PRKCH | CSTA | SGK1 |
| FCRLA | RPL3 | TIMP1 | AC017083.1 | RCSD1 | CD3E | SULT1A1 | SERPINA1 |
| SNX2 | GYPC | PLAUR | PLEC | HLA-DRB1 | TRGC2 | S100A6 | PLEK |
| HVCN1 | PCED1B | LGALS2 | NBPF26 | CD19 | GZMK | PLBD1 | HSP90AA1 |
| HLA-DMA | RPS4Y1 | ATF3 | GK | IGLC3 | ADGRG1 | IGSF6 | KLF4 |
| EZR | OXNAD1 | VIM | YME1L1 | BACH2 | HCST | TNFSF13B | CD14 |
| SPIB | RPL32 | PLD4 | RALGAPA1 | ILAR | IFITM1 | TKT | FTH1 |
| JUNB | RPS25 | S100A10 | ARHGEF40 | HLA-DOB | C12orf75 | AIF1 | ATP2B1-AS1 |
| FCRL1 | RPL5 | GRN | CD83 | LTB | APMAP | FPR1 | CDKN1A |
| CD22 | NPM1 | CFP | ID2 | SWAP70 | S1PR5 | PYCARD | TYROBP |
| RUBCNL | PDE3B | GSTP1 | WDR74 | CYB561A3 | LITAF | APLP2 | FOSB |
| NFKBID | CAMK4 | AREG | BX284668.6 | SMIM14 | CD7 | PGD | CSTA |
| SWAP70 | SARAF | CHMP1B | ABHD5 | CD72 | TRBC2 | CFP | ATF3 |
| BIRC3 | EEF1A1 | ALDH2 | AC004854.2 | HLA-DRB5 | PTPRCAP | LGALS1 | FTL |
| TCF4 | RPS21 | JAML | RSRP1 | BLK | GNG2 | CPVL | S100A9 |
| ZNF331 | RPLP0 | TIPARP | AL021453.1 | EAF2 | CALM1 | FCGRT | AIF1 |
| CD19 | MAL | CTSH | SOX4 | BLNK | RNF125 | FGL2 | JUN |
| IGKC | CD3E | EMP1 | AL118558.3 | ADAM28 | SYNE2 | BLVRB | TRIB1 |

| | Cluster 17 | | Cluster 19 | Cluster 20 | | | |
|---|---|---|---|---|---|---|---|
| Cluster 16 | CD14_6- | Cluster 18 | CD8_Eff- | CD8_Eff- | Cluster 21 | Cluster 22 | Cluster 23 |
| Tregs | LYZ | CD8_Prolif | CD69 | TBX21 | NK-CD56 | pDCs | Platelets |
| IL32 | S100A9 | STMN1 | IFIT2 | TRBV5-1 | GNLY | PLD4 | PPBP |
| FOXP3 | LYZ | TUBA1B | OASL | TRAV12-2 | XCL1 | ITM2C | TUBB1 |
| TRBV20-1 | VCAN | HIST1H4C | IFIT3 | CD8A | XCL2 | JCHAIN | PF4 |
| DUSP4 | FCN1 | TUBB | TNF | NKG7 | GZMK | LILRA4 | CAVIN2 |
| CTLA4 | S100A8 | TYMS | PMAIP1 | CCL5 | IL2RB | IRF7 | SPARC |
| CD27 | S100A12 | HMGB2 | IFIT1 | GZMH | CMC1 | PTGDS | GP9 |
| RGS1 | CD14 | MKI67 | ISG15 | FGFBP2 | CTSW | CCDC50 | HIST1H2AC |
| IL2RA | GRN | HMGN2 | CCL5 | CCL4 | KLRC1 | SERPINF1 | GNG11 |
| PBXIP1 | APLP2 | DUT | CCL4 | GZMM | CD7 | TCF4 | CLU |
| GBP5 | TYROBP | HMGB1 | GZMH | CST7 | KLRD1 | GZMB | MYL9 |
| ARID5B | LGALS2 | MCM7 | ZC3HAV1 | TRAC | SELL | APP | MPIG6B |
| BATF | NEAT1 | PCNA | CD69 | GZMA | KLRF1 | IGKC | NRGN |
| TIGIT | LGALS1 | H2AFZ | GNLY | CD6 | HOPX | IRF8 | RGS18 |
| SPOCK2 | CTSB | H2AFV | CST7 | CD3G | MATK | TPM2 | TREML1 |
| CD3D | S100A6 | PCLAF | HERC5 | CD8B | KLRB1 | UGCG | F13A1 |
| LTB | IL1B | GZMA | FGFBP2 | TUBA4A | DUSP2 | MZB1 | NCOA4 |
| AQP3 | IER3 | IL32 | IFNG | THEMIS | AREG | ALOX5AP | TSC22D1 |
| RTKN2 | TMEM176B | DEK | GZMA | ADGRG1 | IFITM1 | PPP1R14B | CMTM5 |
| SYNE2 | SPI1 | HIST1H1B | CTSW | DUSP2 | SERPINE1 | C12orf75 | TMEM40 |
| STAM | CSTA | CENPF | NKG7 | KLRB1 | MAFF | TCL1A | PF4V1 |
| ISG20 | TYMP | TK1 | IFIH1 | TNF | TRDC | SEC61B | ITGA2B |
| AES | MIDN | SMC4 | DDX58 | ORMDL3 | IGFBP4 | TSPAN13 | GRAP2 |
| LMNA | FTL | MCM5 | CD8A | NFATC2 | EGR1 | SCT | PTGS1 |
| TTC39C | S100A10 | UBE2C | RARRES3 | ITGAL | CD69 | IL3RA | AP003068.2 |
| FCMR | VIM | PFN1 | ANXA2R | HLA-B | TNFRSF18 | TXN | MAP3K7CL |
| EVL | TKT | RRM2 | PTGER4 | PTPRCAP | GZMA | DERL3 | VCL |
| CYTOR | TSPO | IDH2 | GZMM | APMAP | NKG7 | PTCRA | HIST1H3H |
| BIRC3 | CSF3R | NUSAP1 | CD3E | LAG3 | IFITM2 | CD74 | MMD |
| TRBC2 | JUND | RANBP1 | CCL4L2 | CLEC2D | JAK1 | GAS6 | TPM4 |
| CD3E | TALDO1 | HIST1H1E | TNFAIP3 | CD3D | NCAM1 | SPIB | CTSA |
| LCK | RNF130 | CENPM | HLA-C | CALR | MAP3K8 | TRAF4 | RGS10 |
| CD52 | NCF2 | DNMT1 | SCML4 | KLRG1 | BHLHE40 | LRRC26 | BEX3 |
| LIME1 | PTPRE | TOP2A | IL32 | CD52 | TPST2 | PLAC8 | LIMS1 |
| TTN | LGALS3 | CDT1 | SPON2 | SLA | ZC3H12A | CYB561A3 | TLN1 |
| CD2 | GNAI2 | DHFR | CD8B | AL157402.2 | ZFP36L2 | IRF4 | ESAM |
| CORO1B | CAPG | PTTG1 | BRD2 | YWHAQ | GATA3 | FAM129C | TUBA4A |
| SKAP1 | FCGRT | C12orf75 | SP140 | LAT | IL18RAP | CLEC4C | MAX |
| CDKN1B | PPIF | PPIA | CITED2 | CD3E | IER2 | HSP90B1 | TRIM58 |
| HPGD | DUSP6 | RAN | ARPC5L | TBX21 | HSH2D | SPCS1 | MPP1 |
| IKZF2 | PLAUR | NUCKS1 | HIST1H4C | TFDP2 | SH2D1B | HERPUD1 | PGRMC1 |
| CLDND1 | CRTAP | RPA3 | HELB | SPOCK2 | TXK | RNASE6 | KIF2A |
| LAT | CTSD | MCM3 | AC016831.7 | PPP2R2B | NFKB1A | LILRB4 | TAGLN2 |
| PTPRCAP | TMEM176A | HIST1H2AJ | C12orf75 | SUN2 | STK17A | ID1 | PARVB |
| OPTN | MAFB | HNRNPA2B1 | SYNE2 | CREM | KIR2DL4 | SELENOS | RSU1 |
| TNFRSF4 | ANXA2 | DNAJC9 | ETV3 | NR4A2 | PLAC8 | NPC2 | ILK |
| ICOS | PLXDC2 | TMPO | HOPX | CCND3 | SPTSSB | RGS1 | C2orf88 |

| Cluster 24 | | | | |
|---|---|---|---|---|
| Plasma cell- | Cluster 25 | Cluster 26 | Cluster 27 | Cluster 28 |
| CD38 | Erythrocytes | CD4-PD1 | Precursor | cDC1 |
| IGKV3-20 | HBB | TRBV20-1 | PRSS57 | HLA-DPB1 |
| IGHA1 | HBA2 | TRAV13-1 | AC084033.3 | HLA-DPA1 |
| JCHAIN | HBA1 | CD52 | SOX4 | HLA-DQA1 |
| IGLV3-1 | ALAS2 | GZMH | SPINK2 | HLA-DRB1 |
| IGKC | HBD | GZMA | CDK6 | HLA-DRA |
| IGHA2 | CA1 | TRAV8-2 | SMIM24 | CD7 |
| IGLV2-14 | AHSP | IL32 | STMN1 | CPVL |
| IGKV4-1 | HBM | CD6 | CYTL1 | CST3 |
| IGLC2 | SLC25A37 | CST7 | ITM2C | HLA-DQB1 |
| IGHG2 | SNCA | CD3G | SNHG7 | SNX3 |
| MZB1 | SLC25A39 | FGFBP2 | CD34 | C1orf54 |
| IGHG1 | DCAF12 | CD3D | GATA2 | DNASE1L3 |
| IGLC3 | SLC4A1 | CD5 | IMPDH2 | HLA-DRB5 |
| IGLL5 | TRIM58 | ITM2A | ANKRD28 | CPNE3 |
| IGHV6-1 | BCL2L1 | TRBC2 | ZFAS1 | CLEC9A |
| HSP90B1 | SELENBP1 | NKG7 | FAM30A | WDFY4 |
| ITM2C | ADIPOR1 | PTPRCAP | NUCB2 | HLA-DMA |
| DERL3 | UBB | LAT | EGFL7 | IRF8 |
| SEC11C | GMPR | C12orf75 | APEX1 | LMNA |
| PPIB | MKRN1 | MIAT | ID1 | LGALS2 |
| IGHG4 | BNIP3L | PYHIN1 | TSC22D1 | SHTN1 |
| TXNDC5 | DMTN | GBP5 | DDAH2 | S100B |
| TNFRSF17 | FBXO7 | SYNE2 | HMGA1 | RGS10 |
| CD79A | NCOA4 | CCL5 | FHL1 | GSTP1 |
| UBE2J1 | FECH | CD3E | NPM1 | IDO1 |
| SSR4 | STRADB | MRPL10 | TXN | DUSP4 |
| FKBP11 | RNF10 | CD99 | CAT | ASAP1 |
| PDIA4 | MPP1 | GZMM | H2AFY | PPT1 |
| POU2AF1 | FAM210B | SYNE1 | HNRNPA1 | FLT3 |
| SUB1 | GYPC | CD2 | ARMH1 | CYB5R3 |
| SSR3 | BSG | LITAF | SPINT2 | BASP1 |
| IGKV3D-20 | MAP2K3 | OPTN | SERPINB1 | CCDC88A |
| MYDGF | EIF1AY | SH2D1A | HSP90AB1 | LGMN |
| SDF2L1 | BPGM | GALM | BEX3 | ACTB |
| ISG20 | PITHD1 | SAMD3 | CNRIP1 | LSP1 |
| PDIA6 | BLVRB | EVL | NME4 | CADM1 |
| SEC61B | PRDX2 | THEMIS | CD82 | RGS1 |
| MANF | FKBP8 | PDCD1 | ST13 | TMSB4X |
| XBP1 | CAT | BIN2 | RPLP0 | HLA-DMB |
| LMAN1 | EPB41 | TBC1D10C | MSI2 | PPA1 |
| AQP3 | EPB42 | NFATC2 | LDHB | C1orf162 |
| SPCS1 | HEMGN | AES | HOXA9 | MARCKSL1 |
| CD27 | NUDT4 | CYTOR | HINT1 | CXCL16 |
| SPCS2 | IFIT1B | IL2RG | MDK | HMGA1 |
| CD38 | TENT5C | HLA-A | EBPL | TXN |
| SPCS3 | RIOK3 | RGS1 | NPTX2 | HLA-DOB |

### Bulk RNA-Seq

Isolated mRNA from matched pre- and 1-week on-treatment tumour biopsies (n=10 patients, Table 1) were obtained using the Qiagen AllPrep DNA/RNA Mini Kit and sequenced using HiSeq 4000 platform. Raw reads were aligned to the Human Reference Genome hg19 via STAR and the expected gene-level counts were calculated using RSEM. Protein-coding genes with >0.5 counts per million were retained and differentially-expressed gene (DEG) analyses were conducted using R package DESeq2 with Benjamini-adjusted P<0.05 and |log₂(fold-change)|>0.5 (Table 7). Functional pathway analysis was conducted using DAVID v6.8.

TABLE 7
Responders' tissue RNA seq On- vs Pre-treatment differentially-enriched
genes (DEGs) (padj < 0.01 & log₂FoldChange > 1)

| Gene | baseMean | log2FoldChange | lfcSE | stat | pvalue | padj |
|---|---|---|---|---|---|---|
| BAX | 663.3992 | 1.0122 | 0.2030 | 4.9875 | 6.12E−07 | 8.33E−05 |
| SESN1 | 592.7856 | 1.0150 | 0.1863 | 5.4472 | 5.12E−08 | 1.16E−05 |
| FAM105A | 157.5600 | 1.0179 | 0.2847 | 3.5750 | 3.50E−04 | 8.88E−03 |
| SIK1 | 56.7679 | 1.0435 | 0.2313 | 4.5110 | 6.45E−06 | 4.56E−04 |
| CTD-2192J16.22 | 132.6105 | 1.0456 | 0.2764 | 3.7829 | 1.55E−04 | 4.96E−03 |
| FOS | 209.0933 | 1.0516 | 0.2649 | 3.9690 | 7.22E−05 | 2.84E−03 |
| MMP24 | 306.3570 | 1.0562 | 0.2462 | 4.2896 | 1.79E−05 | 1.00E−03 |
| CATSPERG | 111.6319 | 1.0577 | 0.2810 | 3.7636 | 1.67E−04 | 5.22E−03 |
| EBI3 | 68.0792 | 1.0610 | 0.2910 | 3.6455 | 2.67E−04 | 7.46E−03 |
| PFKFB3 | 595.8958 | 1.0645 | 0.1806 | 5.8947 | 3.75E−09 | 1.47E−06 |
| ZNF846 | 56.5537 | 1.0662 | 0.2567 | 4.1536 | 3.27E−05 | 1.62E−03 |
| HIST1H4H | 113.8986 | 1.0665 | 0.2414 | 4.4171 | 1.00E−05 | 6.11E−04 |
| DOK2 | 375.0539 | 1.0697 | 0.2951 | 3.6244 | 2.90E−04 | 7.84E−03 |
| RGAG4 | 27.6257 | 1.0835 | 0.2803 | 3.8660 | 1.11E−04 | 3.90E−03 |
| COL9A2 | 93.2183 | 1.0867 | 0.3057 | 3.5550 | 3.78E−04 | 9.31E−03 |
| SLAMF1 | 18.8105 | 1.0935 | 0.2934 | 3.7264 | 1.94E−04 | 5.91E−03 |
| C12orf5 | 341.7826 | 1.1045 | 0.2427 | 4.5509 | 5.34E−06 | 3.97E−04 |
| C1QA | 3278.2161 | 1.1060 | 0.2893 | 3.8234 | 1.32E−04 | 4.43E−03 |
| ETV7 | 67.3885 | 1.1121 | 0.2201 | 5.0535 | 4.34E−07 | 6.65E−05 |
| IGSF6 | 587.9894 | 1.1159 | 0.2771 | 4.0268 | 5.65E−05 | 2.37E−03 |
| CD74 | 43554.1416 | 1.1245 | 0.3106 | 3.6207 | 2.94E−04 | 7.87E−03 |
| CXCL10 | 802.4861 | 1.1261 | 0.2964 | 3.7989 | 1.45E−04 | 4.75E−03 |
| CARD16 | 247.2755 | 1.1311 | 0.1676 | 6.7504 | 1.47E−11 | 1.67E−08 |
| GZMA | 118.9791 | 1.1321 | 0.3175 | 3.5660 | 3.62E−04 | 9.04E−03 |
| TNFSF13B | 258.4965 | 1.1352 | 0.2881 | 3.9398 | 8.16E−05 | 3.08E−03 |
| ALDH3B1 | 210.0560 | 1.1355 | 0.2329 | 4.8759 | 1.08E−06 | 1.23E−04 |
| LILRB1 | 126.9941 | 1.1398 | 0.3086 | 3.6936 | 2.21E−04 | 6.54E−03 |
| SYK | 532.0813 | 1.1430 | 0.3112 | 3.6722 | 2.40E−04 | 6.95E−03 |
| APOBEC3G | 210.0617 | 1.1546 | 0.2495 | 4.6284 | 3.68E−06 | 2.98E−04 |
| VEGFB | 346.2765 | 1.1594 | 0.2392 | 4.8477 | 1.25E−06 | 1.34E−04 |
| BTG2 | 510.6756 | 1.1607 | 0.1868 | 6.2148 | 5.14E−10 | 2.91E−07 |
| GLIS3 | 64.7066 | 1.1790 | 0.2668 | 4.4185 | 9.94E−06 | 6.09E−04 |
| SLC8A1 | 79.7110 | 1.1922 | 0.3253 | 3.6651 | 2.47E−04 | 7.08E−03 |
| HLA-DRB5 | 156.6299 | 1.1944 | 0.3299 | 3.6209 | 2.94E−04 | 7.87E−03 |
| RND3 | 667.4747 | 1.1985 | 0.1913 | 6.2650 | 3.73E−10 | 2.28E−07 |
| RP11-434D12.1 | 17.6500 | 1.2061 | 0.3227 | 3.7370 | 1.86E−04 | 5.73E−03 |
| EDN1 | 184.0480 | 1.2075 | 0.2958 | 4.0818 | 4.47E−05 | 2.03E−03 |
| LRMP | 109.2794 | 1.2143 | 0.2700 | 4.4977 | 6.87E−06 | 4.72E−04 |
| HLA-DRB1 | 3564.8531 | 1.2180 | 0.3037 | 4.0108 | 6.05E−05 | 2.49E−03 |
| MT1E | 1238.9251 | 1.2193 | 0.2524 | 4.8312 | 1.36E−06 | 1.45E−04 |
| RNASE6 | 302.4286 | 1.2278 | 0.2877 | 4.2674 | 1.98E−05 | 1.08E−03 |
| MDM2 | 805.9141 | 1.2283 | 0.2660 | 4.6170 | 3.89E−06 | 3.10E−04 |
| ZNF132 | 31.7501 | 1.2285 | 0.2870 | 4.2810 | 1.86E−05 | 1.03E−03 |
| RPS27L | 1390.8281 | 1.2296 | 0.2411 | 5.1009 | 3.38E−07 | 5.71E−05 |
| MT1X | 1207.1122 | 1.2366 | 0.2951 | 4.1903 | 2.79E−05 | 1.40E−03 |
| SPINT1 | 584.0293 | 1.2404 | 0.2588 | 4.7922 | 1.65E−06 | 1.69E−04 |
| CX3CL1 | 144.7039 | 1.2427 | 0.3010 | 4.1280 | 3.66E−05 | 1.77E−03 |
| TRNP1 | 108.2802 | 1.2456 | 0.3172 | 3.9268 | 8.61E−05 | 3.14E−03 |
| MRC1L1 | 49.9997 | 1.2543 | 0.3456 | 3.6296 | 2.84E−04 | 7.72E−03 |
| TYROBP | 1468.6237 | 1.2559 | 0.3370 | 3.7266 | 1.94E−04 | 5.91E−03 |
| CYBB | 438.8996 | 1.2710 | 0.2980 | 4.2645 | 2.00E−05 | 1.09E−03 |
| AIF1 | 1053.1524 | 1.2747 | 0.3235 | 3.9407 | 8.13E−05 | 3.08E−03 |
| PLD4 | 40.9704 | 1.2766 | 0.3368 | 3.7901 | 1.51E−04 | 4.86E−03 |
| CXCL9 | 245.5947 | 1.2823 | 0.3449 | 3.7175 | 2.01E−04 | 6.06E−03 |
| GBP5 | 196.6332 | 1.2835 | 0.3591 | 3.5744 | 3.51E−04 | 8.89E−03 |
| EVA1C | 101.0368 | 1.2844 | 0.2186 | 5.8754 | 4.22E−09 | 1.55E−06 |
| HLA-DRA | 15243.5299 | 1.2868 | 0.3233 | 3.9798 | 6.90E−05 | 2.74E−03 |
| ITGA2 | 278.3096 | 1.2926 | 0.2905 | 4.4498 | 8.59E−06 | 5.48E−04 |
| FAM84A | 105.8010 | 1.2993 | 0.3540 | 3.6699 | 2.43E−04 | 7.00E−03 |
| HLA-DMB | 1769.6417 | 1.3007 | 0.3316 | 3.9230 | 8.74E−05 | 3.18E−03 |
| GZMH | 40.7098 | 1.3264 | 0.2812 | 4.7167 | 2.40E−06 | 2.16E−04 |
| HLA-DPA1 | 5520.0804 | 1.3292 | 0.2950 | 4.5063 | 6.60E−06 | 4.63E−04 |
| P2RY13 | 55.3886 | 1.3355 | 0.3674 | 3.6351 | 2.78E−04 | 7.67E−03 |
| EVI2B | 192.5567 | 1.3373 | 0.3155 | 4.2379 | 2.26E−05 | 1.20E−03 |
| TRPV4 | 100.2936 | 1.3456 | 0.1972 | 6.8234 | 8.89E−12 | 1.19E−08 |
| B3GNT7 | 29.3060 | 1.3538 | 0.3637 | 3.7219 | 1.98E−04 | 5.98E−03 |
| PTAFR | 457.8141 | 1.3642 | 0.3614 | 3.7753 | 1.60E−04 | 5.05E−03 |
| PRAM1 | 37.5771 | 1.3645 | 0.3838 | 3.5555 | 3.77E−04 | 9.31E−03 |
| HIST1H2BC | 172.4621 | 1.3662 | 0.3800 | 3.5957 | 3.23E−04 | 8.44E−03 |
| CCDC13 | 11.7632 | 1.3731 | 0.3869 | 3.5486 | 3.87E−04 | 9.45E−03 |
| FDXR | 795.5608 | 1.3813 | 0.2871 | 4.8107 | 1.50E−06 | 1.58E−04 |
| EVI2A | 119.3444 | 1.3833 | 0.3880 | 3.5649 | 3.64E−04 | 9.05E−03 |
| ZMAT3 | 661.9698 | 1.3916 | 0.2506 | 5.5522 | 2.82E−08 | 7.15E−06 |
| GDF15 | 1635.2501 | 1.3941 | 0.3541 | 3.9371 | 8.25E−05 | 3.08E−03 |
| MS4A7 | 922.2858 | 1.4101 | 0.2818 | 5.0036 | 5.63E−07 | 7.88E−05 |
| FGL2 | 323.6713 | 1.4212 | 0.1661 | 8.5548 | 1.18E−17 | 8.69E−14 |
| HLA-DPB1 | 4150.2234 | 1.4226 | 0.2949 | 4.8236 | 1.41E−06 | 1.49E−04 |
| SCIMP | 117.6028 | 1.4353 | 0.2987 | 4.8058 | 1.54E−06 | 1.61E−04 |
| FLRT3 | 387.6721 | 1.4462 | 0.3911 | 3.6973 | 2.18E−04 | 6.46E−03 |
| ADORA3 | 244.1249 | 1.4537 | 0.3378 | 4.3040 | 1.68E−05 | 9.53E−04 |
| TREM2 | 645.3767 | 1.4643 | 0.4070 | 3.5975 | 3.21E−04 | 8.41E−03 |
| PLK3 | 263.6737 | 1.4844 | 0.2997 | 4.9529 | 7.31E−07 | 9.27E−05 |
| DDB2 | 602.0357 | 1.4919 | 0.2569 | 5.8069 | 6.36E−09 | 1.95E−06 |
| TNNI2 | 15.6261 | 1.4963 | 0.4117 | 3.6348 | 2.78E−04 | 7.67E−03 |
| FOLR2 | 1117.8487 | 1.4995 | 0.2626 | 5.7093 | 1.13E−08 | 3.15E−06 |
| C1orf162 | 511.2685 | 1.5086 | 0.3335 | 4.5242 | 6.06E−06 | 4.37E−04 |
| PLB1 | 55.0815 | 1.5128 | 0.3201 | 4.7255 | 2.30E−06 | 2.11E−04 |
| SPATA18 | 185.9195 | 1.5363 | 0.3381 | 4.5434 | 5.54E−06 | 4.09E−04 |
| DEFB1 | 951.3485 | 1.5429 | 0.3713 | 4.1558 | 3.24E−05 | 1.61E−03 |
| RRAD | 197.7549 | 1.5429 | 0.2546 | 6.0593 | 1.37E−09 | 6.49E−07 |
| APOBEC3C | 645.4291 | 1.5458 | 0.2344 | 6.5957 | 4.23E−11 | 3.89E−08 |
| HAGHL | 45.0527 | 1.5526 | 0.3801 | 4.0845 | 4.42E−05 | 2.02E−03 |
| BBC3 | 77.9566 | 1.5738 | 0.2542 | 6.1902 | 6.01E−10 | 3.16E−07 |
| PLAC8 | 40.2177 | 1.6062 | 0.4387 | 3.6612 | 2.51E−04 | 7.16E−03 |
| IL18 | 166.9470 | 1.6185 | 0.3539 | 4.5738 | 4.79E−06 | 3.63E−04 |
| MAB21L2 | 20.1950 | 1.6206 | 0.4211 | 3.8487 | 1.19E−04 | 4.13E−03 |
| MARCO | 256.2664 | 1.6284 | 0.3614 | 4.5057 | 6.61E−06 | 4.63E−04 |
| ADAP1 | 89.3954 | 1.6316 | 0.3307 | 4.9340 | 8.06E−07 | 9.79E−05 |
| HLA-DQB1 | 379.4814 | 1.6469 | 0.3590 | 4.5875 | 4.49E−06 | 3.45E−04 |
| FAM26F | 216.2923 | 1.6519 | 0.2581 | 6.3996 | 1.56E−10 | 1.04E−07 |
| FOSL1 | 36.0891 | 1.6581 | 0.3804 | 4.3592 | 1.31E−05 | 7.68E−04 |
| CYGB | 306.8860 | 1.6605 | 0.4377 | 3.7938 | 1.48E−04 | 4.82E−03 |
| ADRA2C | 22.0475 | 1.6822 | 0.4603 | 3.6545 | 2.58E−04 | 7.31E−03 |
| FAM134B | 334.4171 | 1.6887 | 0.2510 | 6.7264 | 1.74E−11 | 1.83E−08 |
| HLA-DOA | 338.6366 | 1.6982 | 0.3476 | 4.8863 | 1.03E−06 | 1.19E−04 |
| TMEM217 | 16.7564 | 1.7037 | 0.3569 | 4.7735 | 1.81E−06 | 1.80E−04 |
| HLA-DQB2 | 20.6705 | 1.7065 | 0.4760 | 3.5854 | 3.37E−04 | 8.65E−03 |
| HLA-DQA1 | 561.6408 | 1.7079 | 0.3271 | 5.2207 | 1.78E−07 | 3.28E−05 |
| SDS | 1363.8386 | 1.7482 | 0.4709 | 3.7126 | 2.05E−04 | 6.13E−03 |
| RIMKLA | 49.7256 | 1.7581 | 0.4557 | 3.8578 | 1.14E−04 | 4.01E−03 |
| PCDP1 | 69.0836 | 1.7638 | 0.4478 | 3.9390 | 8.18E−05 | 3.08E−03 |
| ACHE | 47.2855 | 1.7746 | 0.4624 | 3.8378 | 1.24E−04 | 4.23E−03 |
| PHLDA3 | 759.0080 | 1.7878 | 0.3090 | 5.7862 | 7.20E−09 | 2.16E−06 |
| FAM180A | 39.7127 | 1.8146 | 0.3189 | 5.6903 | 1.27E−08 | 3.45E−06 |
| PAPPA | 70.3336 | 1.8154 | 0.3523 | 5.1532 | 2.56E−07 | 4.48E−05 |
| GPR82 | 26.4243 | 1.8247 | 0.4477 | 4.0758 | 4.59E−05 | 2.05E−03 |
| TFF3 | 89.6012 | 1.8279 | 0.4765 | 3.8359 | 1.25E−04 | 4.25E−03 |
| HLA-DQA2 | 120.6606 | 1.8511 | 0.3663 | 5.0538 | 4.33E−07 | 6.65E−05 |
| SNAP25 | 52.2102 | 1.8646 | 0.4978 | 3.7455 | 1.80E−04 | 5.56E−03 |
| SIGLEC14 | 14.5514 | 1.8667 | 0.5235 | 3.5661 | 3.62E−04 | 9.04E−03 |
| BEAN1 | 14.2664 | 1.9050 | 0.5300 | 3.5941 | 3.25E−04 | 8.47E−03 |
| MYEOV | 79.2365 | 1.9117 | 0.3468 | 5.5133 | 3.52E−08 | 8.49E−06 |
| NLRP2 | 14.0730 | 1.9138 | 0.4819 | 3.9711 | 7.15E−05 | 2.82E−03 |
| CLEC12A | 37.9246 | 1.9325 | 0.3831 | 5.0438 | 4.56E−07 | 6.92E−05 |
| ZNF812 | 14.5874 | 1.9418 | 0.5054 | 3.8423 | 1.22E−04 | 4.16E−03 |
| TNFRSF10C | 129.0305 | 1.9688 | 0.3405 | 5.7814 | 7.41E−09 | 2.18E−06 |
| LIF | 33.3849 | 1.9770 | 0.2995 | 6.6020 | 4.06E−11 | 3.89E−08 |
| KRT23 | 1490.9715 | 1.9794 | 0.4687 | 4.2232 | 2.41E−05 | 1.26E−03 |
| TSNAXIP1 | 11.8390 | 2.0318 | 0.4332 | 4.6901 | 2.73E−06 | 2.35E−04 |
| SLPI | 768.4440 | 2.0519 | 0.5418 | 3.7870 | 1.52E−04 | 4.90E−03 |
| VSIG2 | 94.0148 | 2.0658 | 0.5781 | 3.5732 | 3.53E−04 | 8.90E−03 |
| EYA2 | 7.5159 | 2.1563 | 0.4720 | 4.5687 | 4.91E−06 | 3.70E−04 |
| NCF1 | 253.0410 | 2.1762 | 0.3162 | 6.8820 | 5.90E−12 | 8.68E−09 |
| IGSF21 | 52.9357 | 2.2214 | 0.3698 | 6.0061 | 1.90E−09 | 8.73E−07 |
| MMP1 | 65.0666 | 2.2350 | 0.4969 | 4.4978 | 6.87E−06 | 4.72E−04 |
| CXCL1 | 236.7692 | 2.2434 | 0.4435 | 5.0580 | 4.24E−07 | 6.65E−05 |
| GCSAM | 8.8915 | 2.2543 | 0.6088 | 3.7031 | 2.13E−04 | 6.34E−03 |
| FCN1 | 206.6671 | 2.2803 | 0.4819 | 4.7318 | 2.23E−06 | 2.09E−04 |
| IL8 | 312.6631 | 2.3135 | 0.4541 | 5.0944 | 3.50E−07 | 5.78E−05 |
| APOBEC3H | 27.0841 | 2.3224 | 0.3932 | 5.9068 | 3.49E−09 | 1.47E−06 |
| PLCXD3 | 46.4704 | 2.3715 | 0.6549 | 3.6213 | 2.93E−04 | 7.87E−03 |
| EDA2R | 89.8673 | 2.4031 | 0.2981 | 8.0613 | 7.55E−16 | 3.70E−12 |
| TRIM29 | 23.7818 | 2.4116 | 0.6022 | 4.0048 | 6.21E−05 | 2.54E−03 |
| LRRN1 | 253.2639 | 2.4806 | 0.4849 | 5.1153 | 3.13E−07 | 5.36E−05 |
| DUOX2 | 43.6972 | 2.5007 | 0.6051 | 4.1324 | 3.59E−05 | 1.75E−03 |
| CLDN9 | 17.8836 | 2.5149 | 0.6999 | 3.5931 | 3.27E−04 | 8.48E−03 |
| ZG16B | 20.2020 | 2.5209 | 0.3924 | 6.4250 | 1.32E−10 | 9.24E−08 |
| LIPH | 30.9054 | 2.5416 | 0.6465 | 3.9310 | 8.46E−05 | 3.10E−03 |
| PTCHD4 | 73.2642 | 2.5662 | 0.5730 | 4.4786 | 7.51E−06 | 4.96E−04 |
| CDKN1A | 8195.3901 | 2.5944 | 0.2863 | 9.0614 | 1.29E−19 | 1.89E−15 |
| GLS2 | 350.3921 | 2.5979 | 0.7145 | 3.6361 | 2.77E−04 | 7.67E−03 |
| PAPPA-AS1 | 15.1223 | 2.6024 | 0.5230 | 4.9757 | 6.50E−07 | 8.46E−05 |
| CRTAC1 | 28.3183 | 2.6139 | 0.6251 | 4.1813 | 2.90E−05 | 1.46E−03 |
| HES2 | 31.9610 | 2.6415 | 0.5409 | 4.8836 | 1.04E−06 | 1.20E−04 |
| CNKSR1 | 16.5192 | 2.6560 | 0.6546 | 4.0574 | 4.96E−05 | 2.17E−03 |
| SGPP2 | 21.4907 | 2.7168 | 0.5730 | 4.7414 | 2.12E−06 | 2.03E−04 |
| VTCN1 | 184.5607 | 2.7224 | 0.7420 | 3.6689 | 2.44E−04 | 7.01E−03 |
| OMG | 26.6902 | 2.7619 | 0.6312 | 4.3758 | 1.21E−05 | 7.18E−04 |
| ITIH5 | 154.7865 | 2.7723 | 0.6526 | 4.2481 | 2.16E−05 | 1.15E−03 |
| DUOXA2 | 93.7765 | 2.7868 | 0.6529 | 4.2683 | 1.97E−05 | 1.08E−03 |
| FXYD2 | 172.1001 | 2.8104 | 0.5676 | 4.9513 | 7.37E−07 | 9.27E−05 |
| KRT12 | 7.9399 | 2.8989 | 0.8129 | 3.5662 | 3.62E−04 | 9.04E−03 |
| EDN2 | 33.4241 | 2.9090 | 0.7913 | 3.6763 | 2.37E−04 | 6.90E−03 |
| CXCL6 | 653.0701 | 2.9739 | 0.6097 | 4.8774 | 1.07E−06 | 1.23E−04 |
| HAMP | 926.3277 | 3.0153 | 0.8096 | 3.7242 | 1.96E−04 | 5.95E−03 |
| CFTR | 241.7644 | 3.0804 | 0.7557 | 4.0761 | 4.58E−05 | 2.05E−03 |
| MMP7 | 7276.0012 | 3.0937 | 0.4365 | 7.0878 | 1.36E−12 | 3.34E−09 |
| TMEM125 | 27.2116 | 3.1198 | 0.7547 | 4.1339 | 3.57E−05 | 1.74E−03 |
| KRT17 | 166.8590 | 3.1236 | 0.4599 | 6.7915 | 1.11E−11 | 1.36E−08 |
| GABRP | 86.2282 | 3.1420 | 0.6312 | 4.9780 | 6.42E−07 | 8.44E−05 |
| CPA4 | 12.3078 | 3.1460 | 0.8658 | 3.6335 | 2.80E−04 | 7.69E−03 |
| CCL13 | 54.0766 | 3.2176 | 0.5414 | 5.9427 | 2.80E−09 | 1.21E−06 |
| CTSE | 45.2973 | 3.2687 | 0.7771 | 4.2061 | 2.60E−05 | 1.34E−03 |
| NRG1 | 71.7372 | 3.4130 | 0.5861 | 5.8231 | 5.78E−09 | 1.81E−06 |
| IGFL2 | 20.7684 | 3.4347 | 0.7361 | 4.6662 | 3.07E−06 | 2.58E−04 |
| CDHR2 | 199.8411 | 3.6076 | 0.7665 | 4.7064 | 2.52E−06 | 2.23E−04 |
| HGFAC | 1255.2645 | 3.7935 | 0.9599 | 3.9521 | 7.75E−05 | 3.01E−03 |
| GDNF | 17.2487 | 4.0021 | 1.0162 | 3.9384 | 8.20E−05 | 3.08E−03 |
| SLC25A47 | 419.3588 | 4.2363 | 1.1749 | 3.6055 | 3.12E−04 | 8.20E−03 |
| INS-IGF2 | 305.0896 | 5.5126 | 1.3248 | 4.1610 | 3.17E−05 | 1.59E−03 |
| CHST4 | 300.3271 | 5.5417 | 0.7107 | 7.7981 | 6.29E−15 | 2.31E−11 |

### Cell Sorting

Peripheral blood mononuclear cells (PBMCs) from four hepatocellular carcinoma (HCC) patients stained with the following fluorochrome-conjugated anti-human antibodies against: CD45, CD3, CD25, CD4, CD8 and CD127 for 30 minutes (Table 4). DAPI was used for detection of live/dead cell populations. The FACS Aria II cell sorter (BD Biosciences) was used to sort the stained cells from each condition into two live immune populations (CD45⁺, DAPI): 1) Tregs (CD3⁺CD4⁺CD25⁺CD127^{low}) and 2) non-Tregs (CD3⁺CD4⁺CD25⁺CD127⁺) with a sorting efficiency of about 91-100%. These cells were then subjected to bulk RNA sequencing (RNA-seq).

### Immunohistochemistry (IHC)

FFPE sections of mouse colons were deparaffinised, rehydrated, and subjected to heat-induced epitope retrieval. Goat serum (DAKO; X0907) was used for blocking. Tissues obtained were stained with anti-mouse CD4 (Abcam; EPR19514; 1:100; OPAL650) and nuclear counterstain, Spectral DAPI (Akoya Biosciences) using the OPAL™ 7-colour IHC Kit (Perkin Elmer). Images were acquired using Vectra 3.0 Pathology Imaging System Microscope (Perkin-Elmer) and images analysed using InForm v2.1 (Perkin Elmer) and Imaris v9.1.0 (Bitplane). CD4 cell density were quantified as number of cells/mm² using average data from 10-15 random fields (0.3345 mm²) with a 20× objective.

### Statistical Analysis

Statistical analyses were performed using unpaired Mann-Whitney U (MWU) or Wilcoxon matched-pairs tests with two-tailed P-values using GraphPad Prism7. Cox regression with Wald test analysis and Kaplan-Meier curves with Log-rank tests were performed using the R package survminer.

### Examples

### Early Immunological Predictors of Response in the Peripheral Blood

Pre- and on-treatment blood samples from HCC patients receiving anti-PD-1 ICB, SG cohort (n=32; Table 1) were analysed using CyTOF and scRNA-seq to uncover the mechanism of response and irAEs (FIG. 2A). An additional KR cohort (n=29; Table 2) was included as a validation cohort and analysed using flow cytometry for defined biomarkers identified from the SG cohort. Further validation was conducted by bulk RNA-seq analysis of pre- versus 1-week on-treatment tumour biopsies (SG cohort) and using a murine HCC model (FIG. 2A). The patients were stratified as: Responders (Res), those who showed partial response (PR) or stable disease (SD) for ≥6 months; and Non-responders (Non-Res), those who showed progressive disease (PD) within 6 months according to RECIST1.1. The 6-month time-point was identified in the Checkmate040 study, in which disease control with stable disease (SD) for ≥6 months was reported in 37% of hepatocellular carcinoma (HCC) patients treated with nivolumab. Patients were also segregated as: Tox, those who experienced Grade (G) 2 and above irAEs; and Non-Tox, those with G1 or no irAEs according to NCI CTCAE v4.03, where G2 irAEs is the point where therapeutic interventions or immune checkpoint blockade interruption would be considered.

CyTOF analysis revealed clusters corresponding to major immune lineages and subtypes according to the relative expression of 38 immune markers (FIG. 2B and FIG. 2C). To identify biomarkers for early prediction of response, pre- and early on-treatment samples (<6 weeks from treatment initiation, before the first restaging CT scan for efficacy determination) were selected from the SG cohort (n=21; Table 1). The initial unsupervised Mann-Whitney analysis of six Res versus six Non-Res clinically matched samples (Table 1) revealed two CD4⁺ clusters: FoxP3⁺CD4⁺ T cells (C33) and FoxP3⁺CTLA4⁺CD4⁺ regulatory T cells (Treg) (C3), and a CD8⁺CD45RO⁺CCR7⁻CXCR3⁺ T_{EM} (C76) cluster that were enriched in responder (Res) group (FIG. 2D, FIG. 2E, and FIG. 2F). Two distinct CD11c⁺ myeloid cell clusters, C4, HLADR^{hi}CD86⁺, indicative of antigen presentation capabilities, and C37, CD14⁺HLADR^{lo/-}, potentially myeloid-derived suppressor cells (MDSCs), were enriched in Res and Non-Res group, respectively (FIG. 2D, FIG. 2E, and FIG. 2F). Validation of these clusters by supervised manual gating with FlowJo (FIG. 2G) confirmed the significant enrichment of these immune subsets (n=21, FIG. 2H). Notably, these clusters showed similar frequencies in pre- or early on-treatment (<6 weeks) blood, particularly in the responders (Res) (FIG. 2I).

The enrichment of peripheral Tregs, CXCR3⁺CD8⁺ T_{EM} cells and APCs in Res, and MDSCs in Non-Res is subsequently validated by flow cytometric analysis of an independent anti-PD1-treated KR cohort (n=29; FIG. 2J, FIG. 2K and Table 2). Moreover, Kaplan-Meier analyses showed that higher frequencies of Tregs, APCs and CXCR3⁺CD8⁺ T_{EM} cells were significantly associated with superior progression-free survival (PFS) in both cohorts (FIG. 2L). Multivariate analyses of these biomarkers with clinical parameters revealed enrichment of CXCR3⁺CD8⁺ T_{EM} and APCs as independent predictors of progression-free survival (PFS) in both cohorts, while (≥G2) irAEs incidence showed marginal significance (Table 8). To examine the influence of irAEs status on the association of these immune biomarkers with response, the patients were segregated according to their Tox status. CXCR3⁺CD8⁺ T_{EM} cells and APCs were observed to remain significantly enriched in Res, particularly in Non-Tox patients, from both cohorts (FIG. 2M and FIG. 2N). These data show that peripheral CXCR3⁺CD8⁺ T_{EM} and APCs are independent predictors of response and progression-free survival (PFS) in hepatocellular carcinoma (HCC) patients treated with anti-PD-1 immune checkpoint blockade.

TABLE 8
Univariate and multivariate analyses for the Singapore (n = 21) and Korea (n = 29) cohorts.

| | SG Cohort | | | | | | | KR Cohort | | | | | | |
|---|---|---|---|---|---|---|---|---|---|---|---|---|---|---|
| | | Univariate | | | Multivariate | | | | Univariate | | | Multivariate | | |
| | | Analysis | | | Analysis | | | | Analysis | | | Analysis | | |
| | | | 95% | p | | 95% | p | | | 95% | p | | 95% | p |
| Variable | N (%) | HR | CI | value | HR | CI | value | N (%) | HR | CI | value | HR | CI | value |

Clinical Characteristics

| | | | | | | | | | | | | | | | |
|---|---|---|---|---|---|---|---|---|---|---|---|---|---|---|---|
| Viral | Hep | 11 | 1.33 | 0.5- | 0.57 | | | | 24 | 0.72 | 0.26- | 0.52 | | | |
| Status | B/C | (52.4) | | 3.5 | | | | | (82.8) | | 1.97 | | | | |
| | Non- | 10 | 1 | | | | | | 5 | 1 | | | | | |
| | Viral | (47.6) | | | | | | | (17.2) | | | | | | |
| Steatohepatits\hat{ } | NASH | 8 | 0.7 | 0.08- | 0.75 | | | | 2 | 0 | (0- | 1 | | | |
| | | (88.9) | | 6.32 | | | | | (50) | | Inf) | | | | |
| | ASH | 1 | 1 | | | | | | 2 | 1 | | | | | |
| | | (11.1) | | | | | | | (50) | | | | | | |
| BCLC | C and | 16 | 1.4 | 0.45- | 0.56 | | | | 29 | NA | NA | NA | | | |
| Stage | D | (76.2) | | 4.33 | | | | | (100) | | | | | | |
| | A and | 5 | 1 | | | | | | 0 | NA | | | | | |
| | B | (23.8) | | | | | | | (0) | | | | | | |
| Sex | Male | 20 | 0.92 | 0.12- | 0.94 | | | | 28 | 0.733 | 0.1- | 0.76 | | | |
| | | (95.2) | | 7.13 | | | | | (96.6) | | 5.56 | | | | |
| | Female | 1 | 1 | | | | | | 1 | 1 | | | | | |
| | | (4.8) | | | | | | | (3.4) | | | | | | |
| Race | Chinese | 16 | 2.1 | 0.60- | 0.25 | | | | 29 | NA | NA | NA | | | |
| | | (76.2) | | 7.41 | | | | | (100) | | | | | | |
| | Others | 5 | 1 | | | | | | 0 | NA | | | | | |
| | | (23.8) | | | | | | | (0) | | | | | | |
| Age | ≥Median | 12 | 0.35 | 0.12- | 0.05 | | | | 17 | 1.79 | 0.72- | 0.21 | | | |
| | | (57.1) | | 1.00 | | | | | (58.6) | | 4.45 | | | | |
| | <Median | 9 | 1 | | | | | | 12 | 1 | | | | | |
| | | (42.9) | | | | | | | (41.4) | | | | | | |
| AFP | ≥Median | 11 | 2.3 | 0.83- | 0.11 | | | | 15 | 0.86 | 0.39- | 0.71 | | | |
| | | (52.4) | | 6.37 | | | | | (51.7) | | 1.91 | | | | |
| | <Median | 10 | 1 | | | | | | 14 | 1 | | | | | |
| | | (47.6) | | | | | | | (48.3) | | | | | | |
| MVI | Yes | 11 | 1.45 | 0.54- | 0.46 | | | | 15 | 0.55 | 0.22- | 0.2 | | | |
| | | (52.4) | | 3.87 | | | | | (51.7) | | 1.36 | | | | |
| | No | 10 | 1 | | | | | | 14 | 1 | | | | | |
| | | (47.6) | | | | | | | (48.3) | | | | | | |
| Child | Class B | 7 | 2.49 | 0.84- | 0.098 | | | | 5 | 1.02 | 0.34- | 0.97 | | | |
| Pugh | | (33.3) | | 7.33 | | | | | (17.2) | | 3.09 | | | | |
| Score | | | | | | | | | | | | | | | |
| | Class A | 14 | 1 | | | | | | 24 | 1 | | | | | |
| | | (66.6) | | | | | | | (82.8) | | | | | | |
| Development | Yes | 9 | 0.22 | 0.06- | 0.024 | 0.16 | 0.029- | 0.03 | 4 | 0.307 | 0.07- | 0.11 | 0.22 | 0.047- | 0.051 |
| of irAEs ≥G2 | | (42.9) | | 0.82 | | | 0.83 | | (13.8) | | 1.32 | | | 1.0 | |
| | No | 12 | 1 | | | 1 | | | 25 | 1 | | | 1 | | |
| | | (57.1) | | | | | | | (86.2) | | | | | | |
| EHS | Yes | 7 | 1.73 | 0.6- | 0.31 | | | | 27 | 4.57 | 0.6- | 0.14 | | | |
| | | (33.3) | | 5.04 | | | | | (93.1) | | 34.9 | | | | |
| | No | 14 | 1 | | | | | | 2 | 1 | | | | | |
| | | (66.6) | | | | | | | (6.9) | | | | | | |
| Multifocality | Multi | 19 | 2.01 | 0.26- | 0.5 | | | | 21 | 1.28 | 0.5- | 0.61 | | | |
| | | (90.5) | | 15.5 | | | | | (72.4) | | 3.28 | | | | |
| | Uni | 2 | 1 | | | | | | 8 | 1 | | | | | |
| | | (9.5) | | | | | | | (27.6) | | | | | | |
| Prior | Yes | 13 | 0.99 | 0.36- | 0.98 | | | | 29 | NA | NA | NA | | | |
| Therapy | | (61.9) | | 2.72 | | | | | (100) | | | | | | |
| | No | 8 | 1 | | | | | | 0 | NA | | | | | |
| | | (38.1) | | | | | | | (0) | | | | | | |

Immune Cell Subset

| | | | | | | | | | | | | | | | |
|---|---|---|---|---|---|---|---|---|---|---|---|---|---|---|---|
| Tregs | ≥Median | 11 | 0.29 | 0.09- | 0.033 | 1.24 | 0.26- | 0.78 | 16 | 0.35 | 0.14- | 0.023 | 2.68 | 0.62- | 0.19 |
| | | (52.4) | | 0.91 | | | 5.88 | | (55.2) | | 0.86 | | | 11.6 | |
| | <Median | 10 | 1 | | | 1 | | | 13 | 1 | | | 1 | | |
| | | (47.6) | | | | | | | (44.8) | | | | | | |
| CXCR3⁺ | ≥Median | 11 | 0.14 | 0.04- | 0.003 | 0.09 | 0.015- | 0.008 | 15 | 0.219 | 0.09- | 0.0014 | 0.16 | 0.038- | 0.0109 |
| CD8 T_{EM} | | (52.4) | | 0.52 | | | 0.54 | | (51.7) | | 0.55 | | | 0.65 | |
| | <Median | 10 | 1 | | | 11 | | | 14 | 1 | | | 1 | | |
| | | (47.6) | | | | | | | (48.3) | | | | | | |
| APCs | ≥Median | 11 | 0.21 | 0.07- | 0.003 | 0.11 | 0.020- | 0.012 | 15 | 0.3 | 0.12- | 0.0078 | 0.24 | 0.063- | 0.0356 |
| | | (52.4) | | 0.59 | | | 0.60 | | (51.7) | | 0.73 | | | 0.91 | |
| | <Median | 10 | 1 | | | 1 | | | 14 | 1 | | | 1 | | |
| | | (47.6) | | | | | | | (48.3) | | | | | | |
| MDSCs | ≥Median | 11 | 3.19 | 1.17- | 0.024 | 3.49 | 0.74- | 0.11 | 15 | 1.79 | 0.81- | 0.15 | 0.72 | 0.25- | 0.55 |
| | | (52.4) | | 8.74 | | | 16.40 | | (51.7) | | 4.0 | | | 2.12 | |
| | <Median | 10 | 1 | | | 1 | | | 14 | 1 | | | 1 | | |
| | | (47.6) | | | | | | | (48.3) | | | | | | |

N: number
HR: Hazard ratio
NA: Not application
Inf: Infinity
CI: Confidence interval
Hep B/C: Hepatitis B/C virus carrier
Child Pugh Score: Class A—A5-6, Class B—B7-8
Steatohepatitis\hat{ }: Analysis done for Non-Viral etiology patients
irAEs: Immune-related adverse events
NASH: Non-alcoholic steatohepatitis
EHS: Extra-hepatic spread
ASH: Alcoholic steatohepatitis
Multi: Multifocal;
Uni: Unifocal
BCLC: Barcelona Clinic Liver Cancer staging system
AFP: Alpha-fetoprotein (ng/ml)
MVI: Macrovascular invasion
Tregs: Regulatory T cells
CXCR3⁺ CD8 TEM: CXCR3-expressing CD8 Effector memory T cells
APCs: Antigen-presenting cells
MDSCs: Myeloid-derived suppressor cells

Peripheral Immune Markers Associated with irAEs

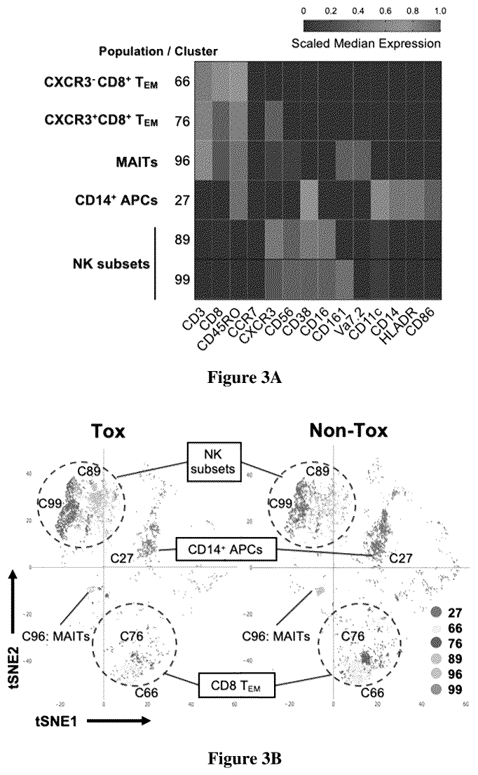

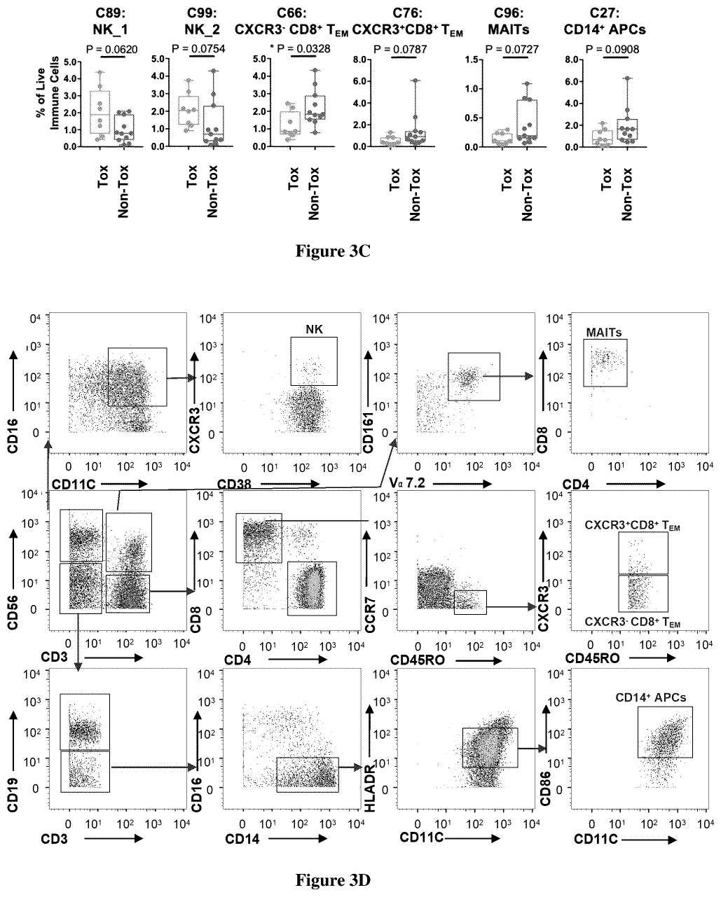

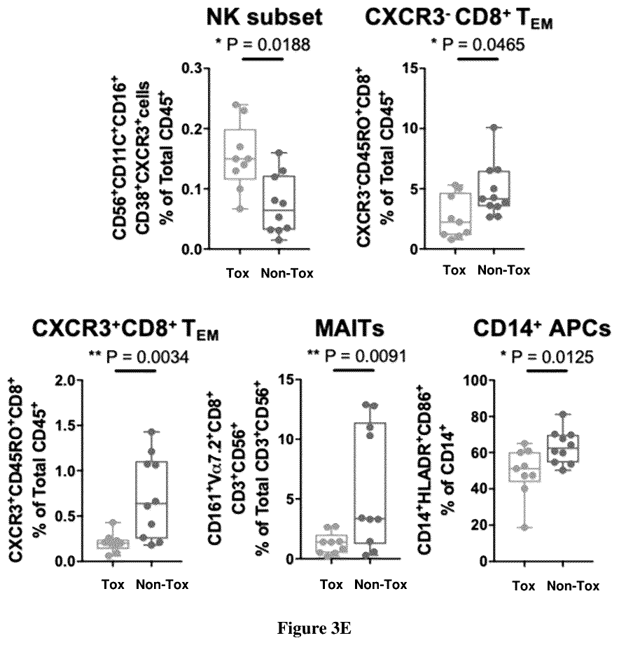

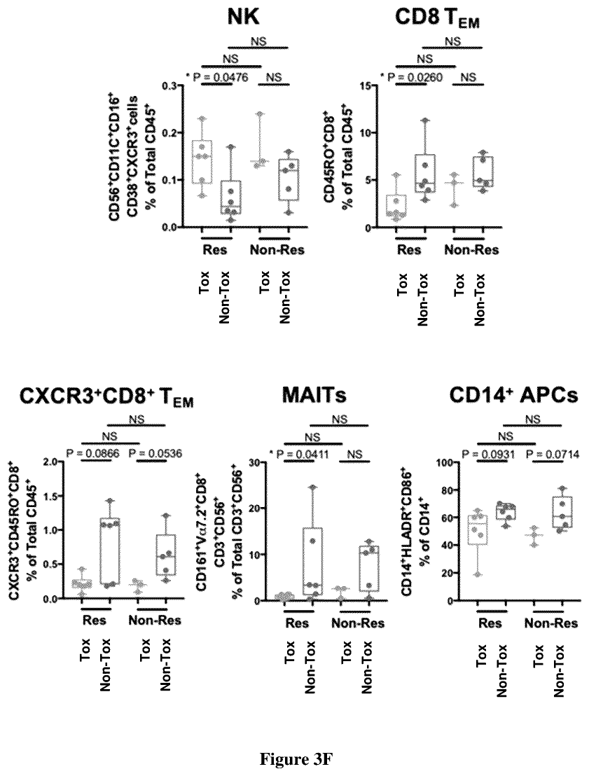

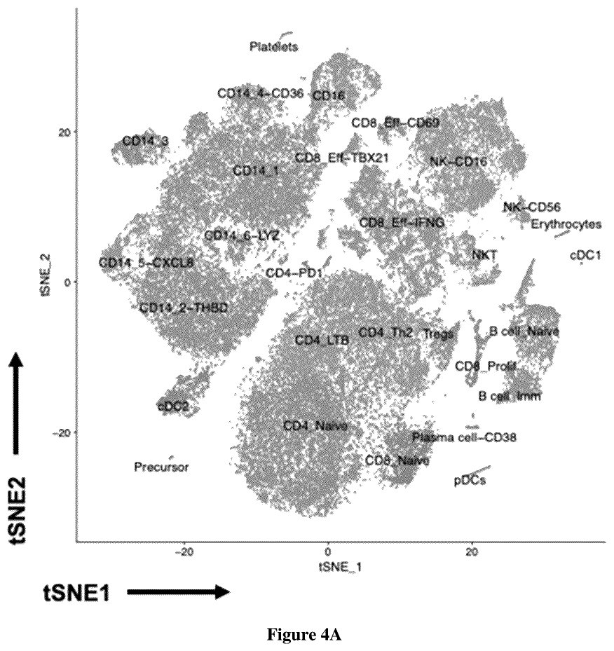

Next analysed blood samples obtained during or close to (±2-weeks)≥G2 irAEs (Tox) versus those at matched post-immune checkpoint blockade time-points from patients who developed no or G1 irAEs (Non-Tox) (Table 1). Due to differences in the study design, this analysis was only performed for the SG cohort. Two CXCR3⁺CD38⁺CD16⁺CD56⁺ NK clusters (C89 and 99) showed enrichment in Tox group (FIG. 3A, FIG. 3B and FIG. 3C). Conversely, three CD8⁺ clusters (C66, 76 and 96): C66 and C76 T_{EM} (CD45RO⁺CCR7⁻) cells and C96 (Vα7.2⁺CD161⁺CD56⁺CD8⁺) mucosal-associated invariant T (MAIT) cells as well as a CD11c⁺CD14⁺HLADR⁺ myeloid cluster (C27), showed enrichment in Non-Tox group (FIG. 3A, FIG. 3B and FIG. 3C). Interestingly, the same CXCR3⁺CD8⁺ T_{EM} (C76) cluster was also enriched in Res as described earlier (FIG. 2F). Manual gating confirmed their enrichment (FIG. 3E and FIG. 3B). All five immune subsets displayed similar trends after controlling for response status (FIG. 3F). Thus, these immune subsets provide insights into immune-related adverse events (irAEs) manifestation, regardless of their response status.

Distinct CD11c⁺ Myeloid APC Subsets Involved in Response and irAEs

To obtain deeper molecular and mechanistic insights into on-treatment transcriptomic perturbations in the immune subsets identified above, scRNA-seq was conducted on 10 PBMC samples consisting of nine on-treatment peripheral blood mononuclear cells (PBMCs) (6 Res versus 3 Non-Res; 5 Tox versus 4 Non-Tox) and one matched pre-treatment sample (Res/Tox) (Table 1). From 59,980 single cells, 29 clusters were identified and annotated according to their respective differentially-enriched genes (DEGs) (FIG. 4A, FIG. 4B and Table 6).

Treg (CD3D⁺CD4⁺FOXP3⁺CTLA4⁺IL2RA⁺) and an APC cluster expressing ITGAX (CD11c), HLA-DPA1, THBD (CD141), and CLEC9A, representing cDC1, were significantly enriched in Res (FIG. 4C, FIG. 4E, FIG. 4F, and FIG. 4H). In addition, two CD14 and ITGAX (CD11c)-expressing myeloid clusters, CD14-1 and CD14-3, were associated with Non-Tox (FIG. 4D, FIG. 4E, FIG. 4G, and FIG. 4H). These CD14⁺ clusters expressed higher levels of KLF4 and CLEC7A, indicating potential polarization to immunosuppressive macrophages.

To decipher the immune mechanisms behind the distinct clinical fates of response and irAEs, we next focused on CD11c⁺ APCs which were associated with both events. The cDC1 cluster enriched in responder group (Res) expressed the highest level of HLA genes (FIG. 4E and FIG. 4H), suggesting superior antigen presentation capability. Indeed, its enriched functional pathways included antigen processing and presentation via MHC class II, T cell co-stimulation, and interferon-gamma-mediated signalling (FIG. 4I), which are important for immune priming. This corroborates other studies associating cDCIs with better survival in cancers as well as anti-tumour roles in adoptive T cell therapy and immune checkpoint blockade.

Comparison of the other two myeloid clusters (CD14-1 and CD14-3) associated with non-Tox group revealed that CD14-1 expressed higher levels of antigen presenting HLA-related genes than CD14-3 (FIG. 4E and FIG. 4H, albeit lower than cDC1 cluster). Conversely, CD14-3 expressed higher levels of immunosuppressive STAB1 (Clever-1) (FIG. 4H). Furthermore, among their enriched functional pathways, peptide antigen assembly with MHC class II and the pro-inflammatory interleukin-1 beta pathway were enriched in CD14-1 but not in CD14-3 (FIG. 4J). In summary, among these CD14 clusters, CD14-3, which is more significantly associated with Non-Tox group (FIG. 4D), displayed reduced antigen presentation/inflammatory characteristics and a more immunosuppressive phenotype than CD14-1.

Distinct Phenotypes of CXCR3⁺CD8⁺ T_{EM} Cells in Response and irAEs

Since CXCR3⁺CD8⁺ T_{EM} cells were identified as the immune subset common to both Res group (FIG. 2H and FIG. 2J) and Tox group (FIG. 3E), we next focused on CXCR3-expressing CD8 T cells (n=863 cells) in the scRNA-seq data (FIG. 5A). Compared to all other T cells, multiple genes involved in antigen presentation, HLA(s), inflammation, granzymes (GZM)s and proliferation, MKI67 were enriched in the CXCR3⁺CD8⁺ T cells (FIG. 5B). Conversely, expression of naïve T cell markers like CCR7, IL7R and LEF1 were downregulated (FIG. 5B), suggesting an effector memory phenotype. Enriched functional pathways included inflammatory response, cytolysis and antigen processing and presentation via MHC class II (FIG. 5C). Thus, these CXCR3⁺CD8⁺ T_{EM} cells display a more inflammatory and cytolytic phenotype compared to other T cells.

Given that the systemic immune landscape is a dynamic ecosystem of immune cell cross-talk that could affect their functions in immunity, CellPhoneDB was employed to identify the expression of receptors and ligands in CXCR3⁺CD8⁺ T_{EM} cells and predict their potential cell-cell communications with other immune cells. Lymphotoxin alpha (LTA) and its receptors, tumour necrosis factor receptor superfamily (TNFRSF) 1A, 1B and lymphotoxin beta receptor (LTBR), which could promote inflammation and oncogenesis, were enriched in both Res and Tox groups (FIG. 5D and FIG. 5E). This suggests that CXCR3⁺CD8⁺ T_{EM} cells form pro-inflammatory interactions with other cells, leading to both response and immune-related adverse events (irAEs).

Furthermore, we observed distinct tumour necrosis factor (TNF) interactions between CXCR3⁺CD8⁺ T_{EM} and myeloid cell populations, where TNF-TNFRSF1B (TNFR2) was enriched in Res, but TNF-TNFRSF1A (TNFR1) was enriched in Non-Tox (FIG. 5D and FIG. 5E). The interactions of TNF with TNFRSF1A and 1B play important roles in macrophage activation and inflammation. To validate the protein expression of TNFα, TNFR1 and TNFR2, we performed flow cytometry on PBMCs from ICB-treated HCC patients (FIG. 5F). Consistent with our data shown in FIG. 5D, we found that CXCR3⁺CD8⁺ T_{EM} cells expressed significantly higher TNFα in Res compared to Non-Res (FIG. 5G). We also observed an increased expression of TNFR1 on both CD14⁺ monocytes and CD14-CD11c⁺HLA-DR⁺ DC in Non-Tox versus Tox (FIG. 5H). However, there was no significant difference in TNFR2 expression on monocytes and DCs between Res and Non-Res (FIG. 5I), indicating that the increased TNF interaction in Res (FIG. 5D) is largely driven by TNFα upregulation; while in Non-Tox (FIG. 5E), it is primarily due to increased TNFR1 expression. This suggests that the different TNF signalling pathways could be harnessed to uncouple response and irAEs in ICB.

Tissue Recruitment of APCs and CXCR3⁺CD8⁺ T_{EM} Cells

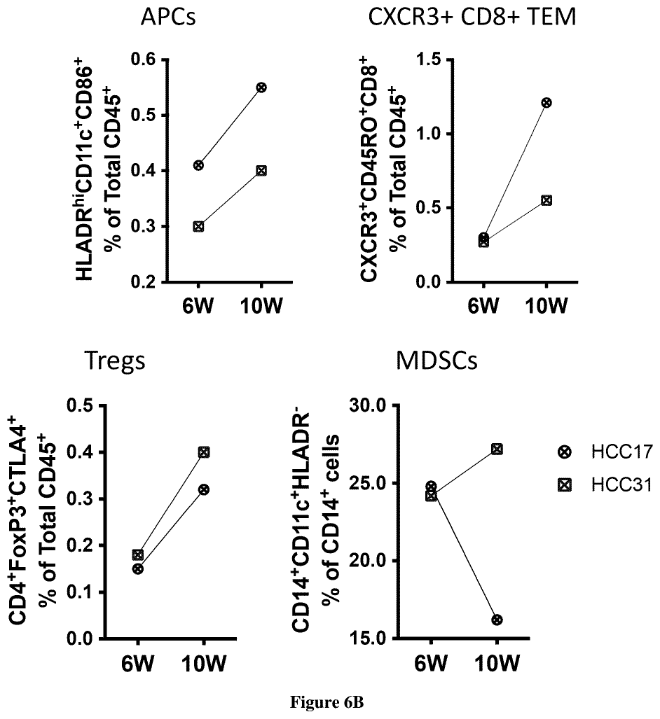

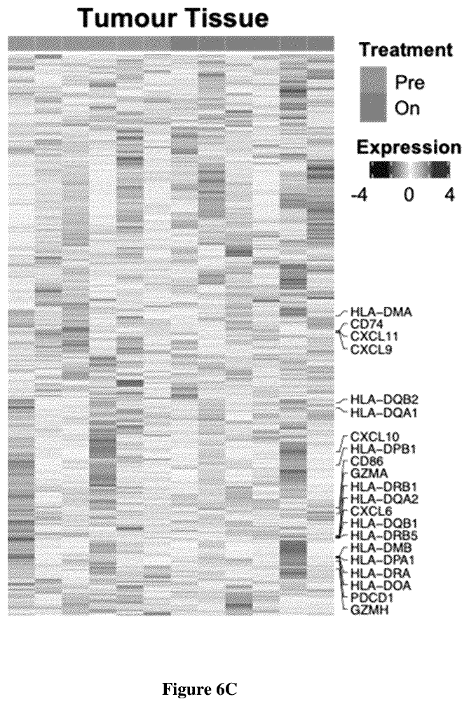

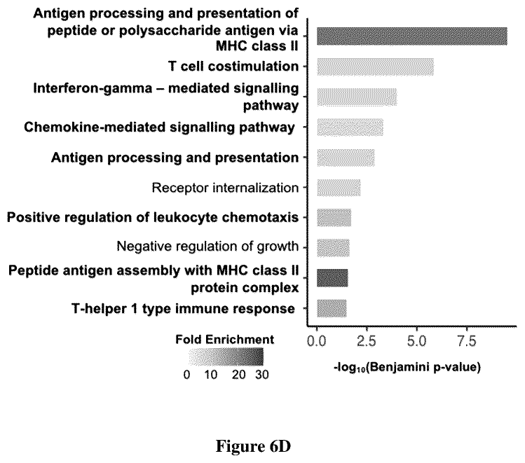

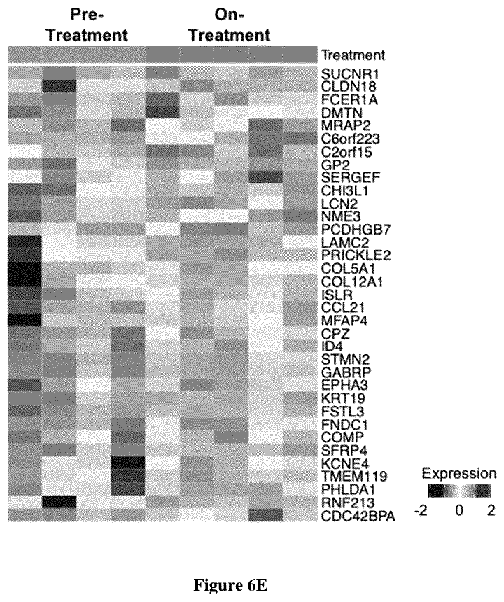

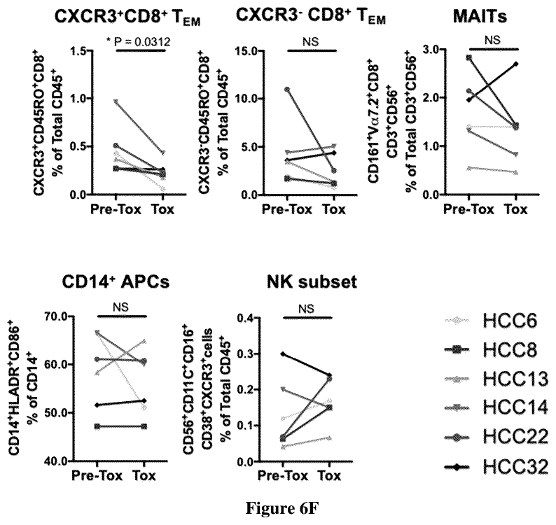

The trafficking of immune cells into tumour tissue for the anti-tumour response induced by immunotherapy could be reflected as changes of their frequencies in the blood. After comparing the frequency of the response-associated immune subsets (FIG. 2H and FIG. 2J) and a significant reduction in APCs and CXCR3⁺CD8⁺ T_{EM} cells in late (>10 weeks) on-therapy blood samples compared to the matched early (<6 weeks) samples is found (Table 1) in Res group (FIG. 6A) but not in Non-Res group (FIG. 6B).

To link our observations in the blood to the events in the tumor microenvironment (TME), bulk tissue RNA-seq was conducted on pre- and 1 week on-treatment tumour biopsies from 10 immune checkpoint blockade (ICB)-treated hepatocellular carcinoma (HCC) patients (6 Res, 4 Non-Res) (Table 1). Differentially-enriched genes (DEGs) analysis comparing on- versus pre-treatment tumours from Res (Table 7) revealed upregulation of genes related to T cell activation (GZMA, GZMH) and antigen presentation (HLA-related genes) (FIG. 6C), the same genes that were also upregulated in CXCR3⁺CD8⁺ T_{EM} cells and APCs (FIG. 5B and FIG. 4E). On-treatment enriched functional pathways from responders (Res) included antigen presentation, T cell co-stimulation, leukocyte chemotaxis, and IFNγ-mediated signalling (FIG. 6D), many of which were common functional pathways enriched in both cDC1 and CXCR3⁺CD8⁺ T_{EM} cells (FIG. 4I and FIG. 5C). These common pathways suggested that cDC1 and CXCR3⁺CD8⁺ T_{EM} cells are recruited to the tumour tissue following immune checkpoint blockade, particularly in responders (Res). Moreover, key chemokines that bind to CXCR3, including CXCL9, CXCL10 and CXCL11, were found enriched in responders (Res) (FIG. 6C), further supporting tumour recruitment of CXCR3⁺CD8⁺ T_{EM} cells in responders (Res). In contrast, non-responders (Non-Res) showed a different set of differentially-enriched genes (DEGs) that were unrelated to immune activation (FIG. 6E).

Since a depletion of CXCR3⁺CD8⁺ T_{EM} cells was also related to irAEs (FIG. 3E), the frequencies of CXCR3⁺CD8⁺ T_{EM} cells in matched on-treatment blood samples taken before (Pre-Tox) and during or close to (±2 weeks) immune-related adverse events (irAEs) (Tox) were analysed. CXCR3⁺CD8⁺ T_{EM} cells were found significantly depleted at the point of immune-related adverse events manifestation (FIG. 6F), suggesting their recruitment to the tumor tissue. These data highlight the importance of CXCR3-mediated migration of CXCR3⁺CD8⁺ T_{EM} cells in the manifestation of response and immune-related adverse events (irAEs).

### TNFR2 Inhibition Uncouples Response and Toxicity to Anti-PD-1 ICB

Single cell RNA sequencing (scRNA-seq) data demonstrated that distinct tumour necrosis factor (TNF) signalling pathways related to Res and Non-Tox (FIG. 5D and FIG. 5E) could be harnessed to uncouple response and immune-related adverse events (irAEs) upon immune checkpoint blockade. This hypothesis was investigated in mice inoculated with hepatoma cells via hydrodynamic tail-vein injection and treated with anti-PD-1 and/or anti-TNFR1 or anti-TNFR2 monoclonal antibodies, twice per week for 2 weeks starting from Day−7 post-tumour induction until Day−21 (FIG. 7A).

At harvest on Day−21, all mice receiving combination treatments showed significant reduction in tumour nodules, especially those treated with anti-PD-1+anti-TNFR2, which displayed no tumour burden (FIG. 7B and FIG. 7C). A significantly higher liver-to-body weight ratio in the mice treated with the anti-PD-1+anti-TNFR1 combination (FIG. 7D) was observed with no significant differences in mouse body weight (FIG. 7E) and reduced tumour burden in this group (FIG. 7C), suggesting liver hypertrophy and inflammation. The higher TNFR1 expression observed in Non-Tox group (FIG. 5E and FIG. 5H), indicating its role in preventing immune-related adverse events (irAEs), corroborates the enhanced toxicity observed in mice treated with anti-PD1+anti-TNFR1 combination. This is further supported by increased CD8⁺ T cells infiltration, especially the pro-inflammatory CD69⁺ activated CD8⁺ T cells, in the non-tumour liver tissue (FIG. 7F and FIG. 7G) and enhanced colonic CD4⁺ T cell infiltration, indicating colitis and intestinal inflammation (FIG. 7H and FIG. 7I). Enhanced toxicities were not observed in the anti-PD-1+anti-TNFR2 combination, which displayed the greatest tumour control (FIG. 7C, FIG. 7D, FIG. 7F, FIG. 7H and FIG. 7I), further strengthening the hypothesis that the differential blockade of TNFR1 or TNFR2 combined with anti-PD-1 therapy can uncouple response and immune-related adverse events (irAEs).

The selective enhanced response following TNFR2 inhibition stemmed from the preferential expression of TNFR2 on highly immunosuppressive Tregs. Tregs and non-Tregs from peripheral blood mononuclear cells (PBMCs), adjacent non-tumour liver and tumour tissues from hepatocellular carcinoma (HCC) patients (FIG. 7J) were sorted and analysed to validate this conclusion. We found significantly higher expression of TNFRSF1B (TNFR2), but not TNFRS1A (TNFR1) in Tregs compared to non-Tregs in tumour-infiltrating leucocytes (TILs) (FIG. 7K). TNFRSF1B expression was also higher in Tregs from TILs compared to Tregs from peripheral blood mononuclear cells (PBMCs) or non-tumour liver-infiltrating leukocytes (FIG. 7K). These findings demonstrated the specificity of TNFR2 expression on Tregs from hepatocellular carcinoma (HCC) tumours, which upon selective inhibition, could enhance anti-tumour response but not systemic toxicity.

Furthermore, intra-tumoral enrichment of CXCR3⁺CD8⁺ T cells and CD11c⁺MHCII⁺XCR1⁺ cDC1 was observed in the mice treated with anti-PD-1, which was further enhanced by the anti-PD-1+anti-TNFR1 combination that corresponded to enhanced tumour control (FIG. 7L). This corroborates the conclusion from human clinical data above, suggesting recruitment of these cells to tumours in responders (Res) (FIG. 6A, FIG. 6C, and FIG. 6D). Notably, the anti-PD-1+anti-TNFR1 combination group, which displayed enhanced immune-related adverse events (irAEs), also displayed a significantly higher infiltration of CXCR3⁺CD8⁺ T cells in the non-tumour liver tissue (FIG. 7M), validating the conclusion of recruitment of CXCR3⁺CD8⁺ T cells and CD11c⁺MHCII⁺XCR1⁺ cDC1 to immune-related adverse events (irAEs) sites (FIG. 6F).

Thus, using this model, anti-PD-1 and anti-TNFR2 were identified as an effective immune checkpoint blockade combination strategy for hepatocellular carcinoma (HCC) with superior response to treatment and reduced immune-related adverse events (irAEs).

### Summary

The present disclosure identified circulating CD11c⁺HLADR⁺ APCs and CXCR3⁺CD8⁺ T_{EM} cells, which are potentially recruited to the tumor microenvironment (TME) upon treatment, as biomarkers for response to anti-PD-1 immune checkpoint blockade in liver cancer patients.

While previous studies explored biomarkers for immune checkpoint blockade-induced immune-related adverse events (irAEs), such as intra-tumoural T cell activation or clonal expansion and circulating B cells, none have explored the immunological trajectories spanning response and immune-related adverse events (irAEs). In the present disclosure, CXCR3⁺CD8⁺ T_{EM} cells were identified with tissue-recruitment capability contributed to both response and irAEs, and demonstrated that local tumour inflammatory cues, specifically the upregulation of the chemokine ligands CXCL9, 10 and 11 upon immune checkpoint blockade, induce their recruitment.

Finally, based on predicted cell-cell communications between CXCR3⁺CD8⁺ T_{EM} cells and other immune cells, distinct pathways were identified involving TNFR1 and TNFR2 that were harnessed to uncouple response from irAEs in anti-PD-1 immune checkpoint blockade therapy. The experimental results disclosed herein demonstrated that TNFR1 and TNFR2 each govern distinct pathways underlying the response and irAEs. The TNF-TNFR2 interaction was enriched in responders (Res) and was likely driven by the increased expression of TNFα on CXCR3⁺CD8⁺ T_{EM} rather than TNFR2. As evidenced in the hepatocellular carcinoma (HCC) murine study disclosed herein, TNFR2 was implicated in immune evasion and tolerance, making it a potential immune checkpoint target and a promising candidate for combination immunotherapy. Moreover, the complex effects of TNFR1 and TNFR2 highlighted the potential of selective TNFR2 inhibition as a promising immunotherapeutic strategy to uncouple anti-tumour efficacy from autoimmune toxicity in combination with immune checkpoint blockade for treatment of cancers.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. A method of treating liver cancer in a subject, comprising detecting an immune cell population that comprises one or more biomarkers selected from the group consisting of CXCR3, CD45RO, CCR7, CD8, HLADR, ITGAX (CD11c), and CD86 in a sample obtained from the subject; and administering a therapeutically effective amount of an immune checkpoint inhibitor to the subject if the immune cell population is detected in the sample.

2. A method of treating liver cancer in a subject, comprising detecting an immune cell population that comprises one or more biomarkers selected from the group consisting of CXCR3, CD45RO, CCR7, CD8, HLADR, CD86, and CD14 in a sample obtained from the subject; and administering a therapeutically effective amount of an immune checkpoint inhibitor to the subject if the immune cell population is detected in the sample.

3. The method of claim 1, further comprising administering one or more anti-cancer drugs to the subject.

4. The method of claim 2, further comprising administering one or more anti-cancer drugs to the subject.

5. The method of claim 1, wherein the immune checkpoint inhibitor is selected from the group consisting of anti-PD-1, anti-PD-L1, anti-CTLA-4, anti-TIGIT, anti-LAG3, and anti-Tim3, and any combination thereof.

6. The method of claim 2, wherein the immune checkpoint inhibitor is selected from the group consisting of anti-PD-1, anti-PD-L1, anti-CTLA-4, anti-TIGIT, anti-LAG3, and anti-Tim3, and any combination thereof.

7. The method of claim 1, wherein the immune checkpoint inhibitor is an anti-PD-1.

8. The method of claim 2, wherein the immune checkpoint inhibitor is an anti-PD-1.

9. The method of claim 3, wherein the anti-cancer drug is TNFR2 inhibitor.

10. The method of claim 4, wherein the anti-cancer drug is TNFR2 inhibitor.

11. The method of claim 1, wherein the liver cancer is selected from the group consisting of hepatocellular carcinoma, cholangiocarcinoma, and hepatoblastoma.

12. The method of claim 2, wherein the liver cancer is selected from the group consisting of hepatocellular carcinoma, cholangiocarcinoma, and hepatoblastoma.

13. The method of claim 1, wherein the immune cell population comprises:
i. a CXCR3⁺CD45RO⁺CD8⁺ effector memory T (T_{EM}) cell population; or
ii. a ITGAX(CD11c)⁺HLADR⁺CD86⁺ antigen presenting cell (APC) population.

14. The method of claim 2, wherein the immune cell population comprises:
i. a CXCR3⁺CD45RO⁺CD8⁺ effector memory T (T_{EM}) cell population; or
ii. a CD14⁺HLADR⁺CD86⁺ antigen presenting cell (APC) population.

15. The method of claim 13, wherein the CXCR3⁺CD45RO⁺CD8⁺ effector memory T (T_{EM}) cell population is a CXCR3⁺CD45RO⁺CD8⁺CCR7 effector memory T (T_{EM}) cell population.

16. The method of claim 14, wherein the CXCR3⁺CD45RO⁺CD8⁺ effector memory T (T_{EM}) cell population is a CXCR3⁺CD45RO⁺CD8⁺CCR7 effector memory T (T_{EM}) cell population.

17. The method of claim 1, wherein the detection of the immune cell population comprises the one or more biomarkers selected from the group consisting of CXCR3, CD45RO, CCR7, CD8, HLADR, ITGAX (CD11c), and CD86 in the sample obtained from the subject indicates that the administration of the therapeutically effective amount of an immune checkpoint inhibitor results in a complete or partial response in the subject.

18. The method of claim 2, wherein the detection of the immune cell population comprises the one or more biomarkers selected from the group consisting of CXCR3, CD45RO, CCR7, CD8, HLADR, CD14, and CD86 in the sample obtained from the subject indicates that the administration of the therapeutically effective amount of an immune checkpoint inhibitor does not result in one or more treatment-induced immune-related adverse events (irAEs) in the subject.

19. A kit or panel of biomarkers for evaluating complete or partial response of a subject suffering from liver cancer to a treatment with an immune checkpoint inhibitor, the kit or panel comprising at least one antibody adapted to target one or more biomarkers in a sample obtained from the subject, wherein the one or more biomarker is selected from the group consisting of CXCR3, CD45RO, CCR7, CD8, HLADR, ITGAX (CD11c), and CD86.

20. A kit or panel of biomarkers for evaluating one or more treatment-induced immune-related adverse events (irAEs) of a subject suffering from liver cancer to a treatment with an immune checkpoint inhibitor, the kit or panel comprising at least one antibody adapted to target one or more biomarkers in a sample obtained from the subject, wherein the one or more biomarker is selected from the group consisting of CXCR3, CD45RO, CCR7, CD8, HLADR, CD86, and CD14.
